SEQUENCE LISTING (18 sequences)

SEQ ID NO: 1 - DNA, 165 bp, Lactobacillus paracasei
  source 1..165: mol_type = genomic DNA
atgaatcaaaaagctttgaatcaatttcctgaacttacctacacagaacaagtgtcggttgttggcggcgatttgtcagtcgaagtcatcatgaaaggtatcttcaccggtatctttgatgctgggtaccaagtgggtcagtcaatcgcaaaatgggttaagtaa

SEQ ID NO: 2 - DNA, 108 bp, Lactobacillus paracasei
  source 1..108: mol_type = genomic DNA
atgaaaggtaagcggcagcacttacttttatattttgttctgggtatgatgacaggactggtgacggcagcgatttttcatatcatttatgcctggctttttcattga

SEQ ID NO: 3 - DNA, 462 bp, Lactobacillus paracasei
  source 1..462: mol_type = genomic DNA
atgcccaaaagggtcgatcaacatatacgttcacgccttaaaggctttactttaattgaagtggtggtcagcctgattttacttgcggcggtcatgctgttatggcgaccggttttattgcatgtcacgcggttcacgcttcaagaccatgtgctaatcacgtcattgcaagcagagcatgacttgcaaatgtttgtacgagataaaaagttgcggtctgtggccttaatgtcggtaagggtgagaagtcccgagaaagcttacacgatcaatttttatcagaccaaacattttcgcggtatggttcgtgtgatgggatctgaaaatgggcatatgccattatttacgcatctaaccggtgtcaattttagcaaggtagctcaaggctttcgctatcgtctgtatttgacgacttcgcagaagattgacggtggggtgcaaatcgatgaagatacgcggtag

SEQ ID NO: 4 - DNA, 507 bp, Lactobacillus paracasei
  source 1..507: mol_type = genomic DNA
atggccgctgatttcacccaattacaacaagccattcgcttgctcaatgcccatactcgagctgctgatgagcaagcgtggcaagtgctttttgatcgttggctggcaactttatcctctgaaactcgccggcaaatgcaaacagttcggtttaatcatgcccaattgacgttactcacaacgctggatcaaagcagtcgcaaacaactgcgcaatcaggatttaaccgctgctgttccgttctcacaaggcctagtctcacgctatgttgctcgccttgttcaattaaacttgctgacaaaattatccttgcccgacaatcgcaaggcctacattgttgcactaactccgcttggtcaacaagtcgctgccttacatcagcaaatgcatcatcacacaaatgctcaactcgcctctgtacttcatacccttgatccacaagatgttcaaactaccattcaggtactcacaaaactgacggctcagcctttacatcccaagtcttag

SEQ ID NO: 5 - DNA, 261 bp, Lactobacillus paracasei
  source 1..261: mol_type = genomic DNA
atgggcggtgtcatttgttacgcggtgccggtcttttggaaaagaatacttcgcagacacctgattcacgagattaagaccctgaatcaaggattgcagttatcaagcaaagccatgagccaattaattgatccggaaaatccttatatggtatttgctgatgaaaatggtgaactggatttttcatttttgtggctaggcaacttgcgtcaattgaggcgtgaactgcgtctaattaaagaacagaaagctagggtttga

SEQ ID NO: 6 - DNA, 1077 bp, Lactobacillus paracasei
  source 1..1077: mol_type = genomic DNA
atgagtaaatataaagttattatttggggattaggaaacgttggtcgttccgcagtgagaatgatcgcggaaagacaaaatatttttgaattggttgcagccgttgacgttgatccaaagaagttaggtaaggatgccggagaagtctttgattttgacaaagtcggcgtcaaagtttcagatgatattgatgcagccttgaaacttccagctgacattgtgctcgacttttgcccaacggaaatggacaaacaaggaacattcatgccttctgctattcgactcgccaaatcgctcgatgccggtaaaaacgttattaccacgattccggtatatcatgttcaagacagtcagccagaagtatatgaatatctaaatgaacatgctaaagcacataatgttgcttttgtaccatttggacttttgccaggcgattatgcctcatatatcccactagttttggccggggccatgggccacgtggataaaattgttgttcaatccggtgaagatgactggcacaacacatcaggctgggtcgatgtcttctcatatggcggcgatatcaataaatatccaaaaccagactcagacgaagatctcttggctaagttcatttatgcttattattcatccggcgtatacgagatggccgataggatcggtctgaaatatgataccttcaaaccagagcatgaagtcttcactgcacccaaagatttggaaacgatcaagggtacagtcaaaaagggcagcatttatgcccacagatttaccatggcactttacaacggcaacgaacaagtagccgccttaagatatgttcataaagttgataataaagagacaccagaattaccgatcaataatacgattcatattgaaggcttgccgtcagtcgatgcgcagatcgatggattgatcccagaaagagaaggctacgtttcatcagccgctccagcagtcaacttgatccctagcattctcgagaccgacaagacaggttatgttgaagtctgcgaccttccagtagtgattgccaggccattggatattggcgcaaaaaaattagtctag

SEQ ID NO: 7 - DNA, 561 bp, Lactobacillus paracasei
  source 1..561: mol_type = genomic DNA
atggcaacctattcgcagatagaactagacataatcaaatcatttaaagggctgatgaaagaccatgaattcactgagatctcaattaaaatgatcgctgaaaaagccgatatcactcgacgcggcttttacaatcacttcttagataaatatgatcttgtcagtaccatctttgagcatgatctttttccaacagtcatcagtttgacgaatatcaatgactgggatcaagggtcgctgtttatcgtgaattatctccaagacaatcgcgactactataaaaaattgttgtcgcttgaaggacaaaactgtttacagacagacttttataaattgactgagatgcagattgggatcttgatcccagaaatattggtcggtaggaaaatttctgacgaagatcaggcatttttaagcgattattattttcacgcttatatgggactgactaccgaatgggtcaaaggtaaatatggtttttcaactcaggagttcgttaaacggtggaaagccttactcaataattcaatgcataattatctggacaactacgctcgatga

SEQ ID NO: 8 - DNA, 1077 bp, Lactobacillus paracasei; identical to SEQ ID NO: 6
  source 1..1077: mol_type = genomic DNA

SEQ ID NO: 9 - DNA, 429 bp, Lactobacillus paracasei
  source 1..429: mol_type = genomic DNA
aaaaacggcttagaacgctcatatttgcgttctaagccgtttttatcagcataggttcttgacaccaataaacatctttagtaattgatcaaatttaggcaatgtgcttttgtcggtgaatggcgatagccctaccgaagcttcagctgaggttcttctgagccacgcaagcgaagcgcgctagggcaagccaacggcgcgcaggcgaagccggagttaaatgtggcgcagccacacctttttagggagcaacgcgaccagaattttgtatggggtttgggaagaggttctccccaaggtcttttgtggttattaacaagcaaaacacaaacacaagcctcgcgcgcgttatatatacttctaaatacttttaaatactttaagtacttagggagacgagaatggctcaaccacgcgtttaaatcgact

SEQ ID NO: 10 - DNA, 241 bp, Lactobacillus paracasei
  source 1..241: mol_type = genomic DNA
acgacctctcgaccacccactgcctcaccaatccccaggtgaaccgggccaagggcactaccgagcaacccgacccctatatcccggtgggcgtggtgaagcagaccgatgggggcatcgtggtgcggggcgcgcggatgctctccacgctgcccacggcggatgagcttttagtcttccccagcactttgctcaaagaagggccgggagccgacaagtacgccgtggccttcgccatccc

SEQ ID NO: 11 - DNA, 7384 bp, Lactobacillus paracasei
  source 1..7384: mol_type = genomic DNA
attgatcgcctccgggtcacttatatgtaactaataatactcccttctctctttagttacaatagggtacagcctatcgaatcacttacgcttccactttgagataacttttcgttattatttatcaacggcattaacgatatcattaactgtttgcaatgcatcgctcagtacactaattggtgcttgttcaatatactgcatgtgtcgttgtacaaaatcaaaggtgtgaaattgtaacggattcacgtacccttctattttttcagtctgaatcggtaccatcaagcctgtttcagctaagcgattatgtttggcatgcgtgatcggacaaaccaacgctaatccagtcattttggcatattgttgattgctaataacgagcgctggtcgtcttttttgaatttcatgaccccgacttggcatgaaatcaatactcacaacatcacctttgcgtggttgataatgcctagtcccactcacttggtaatacctcgttttctaacgtaatatcttgttggtgtacttgttgcttgtaccaatcaccttcaaatggattgcggtgcttcggcagataaagaatgccaccatcatcacgttgctcaactgtaaattcagttccatcggcgattttaatactctttggaatggttaatgtaatggaattgccaacccttcttgctttaactgtcattgaaatcatcctttcgtatacaccgagtatacaccagcgcgaatgaatctgcaactcttgtgccctcttgtgtacaaacaccactgtcaatttacttttgcctattgtgctttatctcttctcgttctgtcattagtatgccaccaacacggccgacttcatccggctcacctttgatgacgccttgaataccatcgatttgcttccaagtctttacttccaagttcttcaaagactttgatatcgtttttcgatgccggcaaacacttcttcattctgatgcctttcaaagaattcctctagatcattcatcagagattctcctcacttaaacctagttggcgtcaaattccatctcagcaatcgaatcttctaggctgtccattacttcgtatgtttcaatgaagttaacccagctgtcatatggatgtagtttgctttctttaatgctagttctaagcttttcgtacacgttgtcaataaactgctcaaccatcctattaaccgcactattaataaccgcttcgttccactgcttagatgctgcacgctttcgattttgactatgctgatgggacagcgcccggcttctgaacaacgctactgccctgctgttagcattcagataggtttcaaattcttcacgatccattacgtttcctcctcaaaataagcctcatttcatagcacagcttcagcaaaaggcatgtcatcctacatgccttttttctgttgctcttcaatatcagtataaaacgtcctgccgctttaggcaaacgtatgttcgctattaagaacatacgtttgtataataactataaaagatttaaaggaggtcaatcgtatggaaaacaatgtcccgcgtgaaaaatggctttaccctgaccgctgcatgaagaaatggctgggctggattctaagcgaccattccgcctatatggaagaagcggctatctcagaacaaccggtgctcccaaagcctgaacagacacaagaaaccattaatggcgtactcgaagatgcttggcaaaactcaaaaattgtcgcagttcaaatcggtacgccatacgatgatcttctgttaccggatattgaaggcgccgtgattggtcattgggacgctcaggtttatctacagcttaaaactggtgagatgcaatccattaatgcagcggacattcgcaatgtgcaactgctcaatccagatcggtggtgggcgttagtatgacgacaccattagatgatccaacaaggttaccggtacacgacattatgtgcattgactgtaagtccttttacgcctcagttgaagctatcagacgcgggattcatccgttagccgccgacattgctgttctcagcaaaggtaattctcctggcggtttggtgctggctgctagtcccaactgcaaaaagcgttaccacgtaggactgagtacacgccgttttcagctaagggacgatatgtaggtagaacttgctgaaccgcggatggctaattacattcgcaagaattacggtatcaatcgtatttaccgtcagtttactgacgatgctcactggtctccctattccgttgacgagtcctttattgacgttacccacccccacaatctcttcggttctaatgaagaaattgctacccaaatacagaagaaggtgtttgatcagtttggcattgtcacaacagttggcattgggcaaaatcccctattggcaaaattagccctagataacgaggctaagaagtcaacgccttggcaagccacttggacttacgatcgtgtgccagaaacaatatggaaacttgatgacttggttgatttttggtcgattggtaatcgaactgccaagaagcttaacgcgattggccttcataatctttacgacttggctcatgcagaccgcgccattctgcaccaaagattcggtgttctcggtgatgccatgtactttcacgcatggggtattgattactcagacttaacccgccgctacttaccacgggccgaaaataaaggctacggcaatagtcaggtactcatgcgtgattacactcaggcaagggagattgaagtcatgcttagcgagattgctgatcaagtggctggccgaattcgccatcaccaagtccaaggtgaggtcattagcgttggcattggttatgctgatgcagaagaagctggcacctccggtttcggtgcgcaaatgaagattgatcccacaaatcgcacagacgatttaattcgcgctactcgatttctcttccatagtaagtggaacggacacgctgttagaaatgtctcagttcgcgttaatcgcatcagccaagcaagtacaatgcaacttagtctatttgaatcagcagagaaggaggaagcaaacgcggctcctatgctgtaattacggataaaagaatcaccatcattaggtttttcgtctaacaattttaggaaacttcactttctaggtcgtaactttatttttgcaatctagggttttttaaatatatacattttagctcgtttgtgtttaatattataatcacaactataccaatgataaatgtctaacataaatatacaaacatgttgacagaagctcttgaatacgtttacaattatttcgttcaggcgagctttgtttttgaaaaagtattaatacaagataactaggttagtggctgttgaattaggcccccgatttcgggaccacgacagtcacttgatactcgatttttatcgtttgctggcttgatcgtacattgaacgaaattggtacagaaaaaagagctaagagccgctccaaattagccaaaacgattgcggcgtcaatgcttacggcgatcgtttctgcagttttagctgttaccagatcaagtcctagtttccctttgatgaaggcaaactcacgctcgatctcacctcgtcgattttcggcttgtcggtctgccttacgtttggccggatcgacctgcttcggccgacggcccaatctaggaccgctaagtttgatcccaagatctgcgcacagcccgatattcgcccgagtccgataaagcgtatcagccaagatctcatccgggtatgtaccatacgtgtcaaaataatggtcgatcgttgctggtaagtcagcactttcgttaaacgcattgaacgcaaaccgttcaacggccacgacgccatgactgatcgatacgtcgatcttgggcccgaattcgaccggatcctttgctttgccgcgaatgatcggtcggatcgctggttgatcaaggcttacgatccgatccgcgactcggtgagtgtgctgtcgatacatttcagtttgttgctcatacaattttcgaatgatcgttaatcgttgtgtctgccgttgattcaattgcccgccttgtgcttgcagttctttgacgtaacgcaagtcacgtcggatgtactgtaattgagccttgatctgcttatgggtcgttttcacccaacggcggggtttacgtgaaaaggcggtccacgtttggtgggccttgcgcttataggtacgcggcggtttgaccgctaattgcttggccatggctgcgatgaatcgctctaaattgagccgcgcctgattgagtagctgcgtatcctgcggatacttgatctttactgggaccgcagtcgcatcagtgatcaagatcttctgatggccaagtttagcttggaggcgatcgcggacaaaatcgctaatgatgttcgtgatcaactcggaaagcggcgcgatccggcgcctgaaataggacagcaccgaaaatgaaaacggtgcttgcggctgatactctggcaggccaataaaatactgataagccggtgtatcgcggatcgctgcgactaactcacggtccgatagctgagtgcgctgcttgatcagttgggcgccataaagcagccgaaagggtttacctgcccatcctaagttagacgggaaagccaattggtacgcctcttctagttgcggccacggaacttggtcggccagttggacccactcgttatctggacttaatggggtgcttaagccgctaccaaacgatttgatcgataattgaacggcttttcgacgataaaccatgatccatgcctccgatagggtcgtgtcaaatgcaaacgaaatgagcacgatccgtaaattcatatgcattcattatacgacgataacgggttcaactcgcatcaaatgtggttatatcaaattattcaacagccactaggttaagatcttcatttaagtgatattcatttgcaagcaattgaaaattactcatcacgaagaggatttcattggccatattggatagcacgcaaatcacttgctttaagaaaatcagttcctttaatgagtctcttaaaggacggggctctcacttgtactcacaatcaatgttaactggagatcaacaatatggtcatagattcccataataacattgacttgactatctaaaagaggcttctaactttgatattggtggggttattggttgcttggctgtaagcagataatcttaacttgggttattttcattgtgttgtaaagacatttgttataaaggccgaagttatcgctttgacttgtaataaattatttttgattgagatatcagaaaataaacgggggataataatgaaaaagattattaggattgttctttgtgttgttagttgcgttagtatcatggtcggatcgcttgggttctattcaactccaaagatcgttaaagccgacagtacatctgttacggatgtcgacattaatacctatatttctagcatgacacttgatcaaaaaattggacaaatgtttgtagcacgaacctcacaagatactgataaagctcgtgctgatatagcaaaatataatcttggcgggctgattgtttatggtgttgatttcactagtgttaaagggacaacagctacagaagctcagaataacttcaagatgaagatgcaaggctttcaaaactcggcaagtctgccactattgattggtgttgatcaagaaggaggggcagtctcacgcttatcacaaaatcctctaattgccaacggcagaagttttccttcaccacaaatggcttatgctaatggtggaatgaccaatgtaacaaaagaagctagtgaagtcggaactattctaaaaaatctgggcattaactggaactatgcaccagttgccgacagtacgcctgacacctctagttttatttatggtagaacctttggtcaagattacttggctactgcaaactatattacgaatgtgatccctgcgtggcaaaatgctggcattgccgcaactctcaagcatttccctggttatggatccgcgattgatacgcatacggattttgcagtcgttacaaagtctaaggaggattttgaaaaagaggacttgcttccctttaagtccggtattacagcaggggcagattctgtaatgattgcacatatagtaatgcaagctgttgacccagtgtatccagcatcattatcacggaaggtcgttaccgatttgttgcgtaatgaacttgggtataatggcttaataattaccgatgcattggaaatgggggccatcaagcaatttgctcaagaacatgatcaagttcctgttgatgttcttgctgttgaagcagggaatgattgcatcatgaataacgattatgaaaccgctattccacagattcatgcagcagtaactaatggaactattaaggaatcagaaatcaatgaacacgttttccgtattcttgatctcaaacgcaaattagggttgttaactaaaggacaacttcagcaaaaaaaagttcaggttgacaatgtttcctacagcagtgacaacaaaaaggcaactgtgagtggaacagttgttgatagtgattggcaagttggagaaccattatcggttaaagactcgactgggaaggtcattattaccgcagacgttggtgccggtggtaagtttactttcgatgttcctactaagtcccaagaacaagtattaactctgactactaatttacccaacatcgctgattctcaaataactattaaggctgtgagttcatcgaatactaacaaagctttgctagaaaacttgatcaacgctgctgaacagttggatagtaatcaatatactgtcaagtcgtgggaagaattacaaactaaactaactgaatcaaaatcgattctgaacaatgatagtgctacacaagatcaagtagacgcttccgttaatgctctacaaattgcccttaagcaattagttcctgtatcaaatagcggaaataatggtcaaagctctaatgatagcagtaaccaaagttcatctagcagtagtggcaaagaatcatccagcaatagcaatgccaatattactagtaaggatcagtcagctaaggattcaaatacgaggcctaaagaccatagtcttttgccaagtacaggtgaacgggtgatgacgggaatttctgttctaggggtaattttaatagcttgtgtgactatattatatattcggaaaaaaggacgcagcttttaattagtctctgcgtcaactggcgttaaaaactagattgaagtaataaagttaccacctggaaagaggcatgctcattgcttgcaagggtgtcgacgtgtaatagaaaagttgggg

SEQ ID NO: 12 - DNA, 1410 bp, Lactobacillus paracasei
  source 1..1410: mol_type = genomic DNA
tggcgtgggcaacgtgcacgttttctagtcgctaaactgtgaacaatgctcgtgctaaatgcaaaactgagcaaggagatgaactataagcgggggaccctttgctattgaggaggaaggcgaagtagagaaagagcggtgatttgaactcgaaacagcggcgccgcaggctagcagcactgttagattaatcgccagcagcagatactagacagcttcttaaaggcttgataatagcgttgcgccatttcaatggaactagtggtcaaaatcgcattgtagttgccatgacccaaactcgttttacgcgggcctttttgtaaaatatattgaacaacttggttaatatgttcatcggtctcaaagtcagccggagttaaatacgtttcttccaagtccttcactgacatcgcttgaatcttggcttgaattttcgcttcatcagccgtactcagcaggcggccttgcttatcacgacgagctttaacgcgctgagtttctttttcaagggcccgggttattaaagcgtctttaccaatcgttgtcacgtgttccacattaaatggtaacactgcttggtcctctaaggcgtctcgcaagttataaacatgacagactttaccaaatagctcctccgtcgtaactgctagatcacctttgagctgtttcttattttcattaaaaatgggggtgccagtgtaaccataccagttactattgataaacgctgctcgaatttccttttgcatcttaccaaactgcgaccggtggacttcttcaacaaagaagatcacccgttgctttaaagtcttactaaagcgggattgcttaccggttgccagctggacttgcgtttttttgaccgcccgatggagcttttgaatcgaggtgaccaagaccttaccgtcattttgttgcaatttacgcattaaatcaccggtgttttgggcttcgttaatggcaatatcatcattggcagcataggcactaaagttgctggttgtctgttcgtctaaatcccgccggtcaactaagaagatgaccttatcgacaccaggatcttgcgcagctaatttagcggttttatatgaggtgagtgttttaccagaacccgtggtatgccaaacgaaaccatcctgatggtcatgaatccggtgcatcacggcttcaatcgcataaatctggtaaggccgtaagagaattaagctttgccgctcttggtcgatgactgtatattcactgaccattttgtgggccatgggaatattaaggacttggcgcgtgaacgctaacccgttttccacggggtgattatcccgcgtccgccaattgaacaaaaaggctttattgaaatgatccggttcggcattcgcaaaatacgccgtactatccggcgtcataatcacaaac

SEQ ID NO: 13 - DNA, 592 bp, Lactobacillus paracasei
  source 1..592: mol_type = genomic DNA
cgatctaaaagctaagttattttccaagatatcaaacaacttcttaacccaagaatcttccacacataggacaataatgaatccaaatagattcagctttttcctgcaaaccgggatcagtataaacgtccagtaccggataatcacgcattaagttcagctgccaatgggtatcatctaaattaaaaagatccgatttagtgtctccccttactacattatggcaatacacacaactgttgttatacatgcttccttgctttttgattttaaactcctccattttgcatattataagaagattacttctacttgatatatagatgctttccttgcgagggtaagtcagacaaggaagcatttctaacttgagatacttaagcttgtctcaatagatgtagatagcggctccccaatcggatattaacagctcaactagtcaaaccagatatataaatgtgacacaagctggaatatatatcattatctagataattcaaattgagctaataaaatcaataaagaaaattttaaataacattattttataaacccctttaggattttcccgatttgatattctacgtatgtt

SEQ ID NO: 14 - DNA, 2233 bp, Lactobacillus paracasei
  source 1..2233: mol_type = genomic DNA
gagtatccaaaaatacgacgggtatttgaataggatacttattaagcgagaatggtattggaaatctgtggcagccactcagcggaaccatacctttatcccaaccccacgcaaaaaaaacatcaagtaatccgtcagatatgatgacttaattgtgggacagttctaatatgaagaaaacaggttagataattggggtgaaaagatggcaacctattcgcagatagaactagacataatcaaatcatttaaagggctgatgaaagaccatgaattcactgagatctcaattaaaatgatcgctgaaaaagccgatatcactcgacgcggcttttacaatcacttcttagataaatatgatcttgtcagtaccatctttgagcatgatctttttccaacagtcatcagtttgacgaatatcaatgactgggatcaagggtcgctgtttatcgtgaattatctccaagacaatcgcgactactataaaaaattgttgtcgcttgaaggacaaaactgtttacagacagacttttataaattgactgagatgcagattgggatcttgatcccagaaatattggtcggtaggaaaatttctgacgaagatcaggcatttttaagcgattattattttcacgcttatatgggactgactaccgaatgggtcaaaggtaaatatggtttttcaactcaggagttcgttaaacggtggaaagccttactcaataattcaatgcataattatctggacaactacgctcgatgaattacacagattggattaaatgagaaagatgttacatttgtgccaatatgtgaattgataaatatttcacaaggaactattctttccctgtaaacgaaagttgacttgaaaggagttagttctgatgagtaaatataaagttattatttggggattaggaaacgttggtcgttccgcagtgagaatgatcgcggaaagacaaaatatttttgaattggttgcagccgttgacgttgatccaaagaagttaggtaaggatgccggagaagtctttgattttgacaaagtcggcgtcaaagtttcagatgatattgatgcagccttgaaacttccagctgacattgtgctcgacttttgcccaacggaaatggacaaacaaggaacattcatgccttctgctattcgactcgccaaatcgctcgatgccggtaaaaacgttattaccacgattccggtatatcatgttcaagacagtcagccagaagtatatgaatatctaaatgaacatgctaaagcacataatgttgcttttgtaccatttggacttttgccaggcgattatgcctcatatatcccactagttttggccggggccatgggccacgtggataaaattgttgttcaatccggtgaagatgactggcacaacacatcaggctgggtcgatgtcttctcatatggcggcgatatcaataaatatccaaaaccagactcagacgaagatctcttggctaagttcatttatgcttattattcatccggcgtatacgagatggccgataggatcggtctgaaatatgataccttcaaaccagagcatgaagtcttcactgcacccaaagatttggaaacgatcaagggtacagtcaaaaagggcagcatttatgcccacagatttaccatggcactttacaacggcaacgaacaagtagccgccttaagatatgttcataaagttgataataaagagacaccagaattaccgatcaataatacgattcatattgaaggcttgccgtcagtcgatgcgcagatcgatggattgatcccagaaagagaaggctacgtttcatcagccgctccagcagtcaacttgatccctagcattctcgagaccgacaagacaggttatgttgaagtctgcgaccttccagtagtgattgccaggccattggatattggcgcaaaaaaattagtctagactaggctttcgaagctgctttgaccattaaggttggagtagctttttcatttgcaagtaaatcattacggcttgtgtatacggtatacaaaatggagaaaacgctgactagtttataaatcattgagacttaacggccggataaatgctgatctgattatagaaataacaacaaaaaggccacgctaaaaatcatattaattataatcgggaaatttattaataatattcaagaaaaataaaaaccgtgggtacattatttaaaa

SEQ ID NO: 15 - DNA, 5866 bp, Lactobacillus paracasei
  source 1..5866: mol_type = genomic DNA
tttgaaactaagacgaaagctgccatgtcaaacaaagccgccataaatgccactgtcacagatccatcagccgcaatgccagcatcttgctgaagttctttaacagcattaagggtgttattcgtgaactcattattaaagtctactggactaatccctttgcaccagaaagccccttgaatgagttgagcaatgttccccttatatcctggcttcagactacctacaacaggtgctaaggcgtttttggtcgtctcgccaaagccttcaccaatagcactaataccgatttcgtgctgtaatcccattcttaggctataaattgttggccatcccgtttgcccgttttctggagctgcgacaaagccaggaacgctaccatacgttttgttgagccatttttgaacggctcgtactgcttcatctgccattttaaagtctccttttttgttttagacagcacgtctgccgtcacaaaaagcaaacatatgttcggattcatttcatctcttcaaagcttcgaaaggcaaccctgatccacaaataatccttttattttgaacttagcaaaaaaatgaggccctcacatagtgttgaagttgcctcattcttaatgtctatatttaaagtattgccacaacgatggatcatcgaacgctcatggacttggttagacatgtatcgacgactatgaaaatgtgggcgcaagctcaatttcaccttccaaatgtttgtgctatctcatttagcgctggtttttttaggatagactagacaaggactaataatttctcaagaatcccgcaactccactattcattcgtcgaaatccccactgatactcttgtccttgcacgttcgaccaagcaagaatgtttattccgataaccgaattgttgccatcaagtaatggacctcccgacatgccatgatacgaattaatttgttgtgaaatatatattcctagtgggtcctctgaaaagggtgtgacagtcccacttgattgaaccatgactccttgaagttcgtaccctgattgcggatcgccagggaatccaatggatcttgctgccatcgtatcagcagggttcgtatttaaattaagacccgcaggcatactaccagacttcatagagacaattgcagccccgtaatcattcgaagtagctgttgaattattaatccatgcctgtggcactatcaatctattcaatactccgtaaccgaccccttgatgatttgcttgactatcaccaaagttaataattcctccagaaatataatgaccatcatataacatgtgtgctgctgtccctatacggtctactccaatgctaaatccagtaccgccagaagttccactgctcagctctgtgccatttgagttagagatgatttttttatacgaactgtcaaggtatggtgagttaatgaccatgacaccatttgccatcgaaaaccacgtgctcaaaaccccaacggaactgtacggcgccgaattggggtttgacacaggtgataccgtcctcacagatagatgattagacgatttattcagtttcgcaagatattcggacgtaattccttgaactttctttccttcttttaaatcctgatattgatcaggtgtataagctttgacactccctttaaaatcgggataagaatactgatattgtcgaatgatttcattcaaaaactgttgtgttgtcgtctggttagtcaaaacaatatcattctttggtaaaacatggctattcgctgccaaaggatttgccatactatcagcacttacacttaccgtttgaacttgaattatcgctaaggctgcagctatcatagtgatatatgcccatacttttcgcaatttaattccccctttttcttaaaatgaaaccgcattcacggaggcttgtcaatgcttttaaaaaacaaacgttacttttggctcatcttggctgtcagcataattggagtaattgttcttgcggtgttatggcgcatgaaccctgaaggaacggcctcaaataagtttgaacgtcccaccattactattaaaaaagtcaaacttattaagcacagtaacagtattgctgtcacatttgctacctctccaaaaagcaaatatacgataagtgatcttaaagagaatcaactttcttctggcatttcgaataaaagagaaaataccgtttcggaattaaagccctcctcctctaagcttgcaatacgggttaagcataacaataatatacaaaccaaagtggtttctgttcccattggttatcatattatgaaaagtgccatttcaagaaagccaattcctatgggagaagagtttaagtacaatggaaagtcgcatgttttatttagcatgaccattacccctaaaaaacaaaacaagaacagtataaaaaacaccactgcttttaacataacggttaaaaatgatcactaccttgtccctgtcgtattagataccaaataccttactgtttctgattcagaaggtaactcattaaaagtaaagccattctcaaaaatttctattccagcaaaaaagaaaaagaccattgcaataactattgagggcgttcccgcaagctctgccaatggtctagttataacgtataatactgtcgatttagacttaccaatctcctttataaattcctgaaattacactaactgtcccccaccttgacagtcagtacactcaaactgtctcttatgcttacaaacacgtaatttaggcggtttttaagcaaaagtcgttagttttcataaatgttatcttatactctaatgagatctagcttgtgataataaggctgtttttctttgacagccttattaagcacactaatcaatgtcaattcgaagtttttggtttcctacttggccaactttgttatcagaaattccaaaactcattgcctcccgccaccatatatttatcgagccattttgaaaatgaaaaatcgaaatatcggtctgcttctattccgggatgagttagatatgattttcctaaccgatacttctctatatcaatatacatatctccgacatcccttatatggagaatgggtactttatttacaaaacttttaggcagtgcctctttttctttaatcatttttctaattgaatagacttcacacgtatatcccataggaatcccttcgattgttgtgtcaaataaaaatagtccattagtaatcgagagaaactcaatgtaatcctgtggaaggttccacctttttattttttctatatcatcagcgtgtgcaggaggttctatcttaaaagaaacattttgcacatctccatctaattggaatgttgagagcgctttttccccatttttcgtcacctttattaaagaattaattcttcgccgaattagagattccaaatgagttcctcctcaatagttgttaaaccacgcggtgatcaaccgatgatttggtgttaacaccggcatcaaattattaaagtcatttgttccgccataaactctcgggcgaatatgatgcacttctcgagaactccaaaaatctgcagattgattgccatatgtttcactgaacgttttaatataaatatatctatcttttgatgaccaactaggagattttgataacttagtccaggtagtattaataggtgtttctgcatcttttttagacacgggatcaacatattcagggaaattctgtcctattttattttgtaaatagatggccgttggtgggattggttttacattagccgctccattaactcctataaatccaacagcgccagcaacgttaaaaaatgtggttttggctggcaattctgtaaaaccaagtaatcctgtttgcaaaccgcctattttgctaatgggtgccagcgtattggcattcaccggacctgcaagggtaggctcagtattaaattcaatttgcacatcaactgcggctggcgggacaccctcaatagaatcaatccagaaattgactctgaattttccggatatgtattcttccgataagtgccaggtaatatttgtaacaggaacttctgcacgagaacttatgccattgccatgactattattcagtaccacttgttttccagaagcgataattggttgtccattttctggattacttcttgataacttgtcagcattggtcagttgagtattgttatcgctaatacggctttcagttgaaacaatttgatttaaagattgtgttgtgtctgcggatacaattgttgaatacccaagcaaattgactagaaagagcccgaatattagcccaataactttccactttttcacgcctattatcttctttccaaagttcttcagtgcctggcaataactgtatacattgagcagtatagtcgctattttatagctgaacaactcataaagctcaattattattagcctataaaaccactgcctaagtgaattgatctagaacgaagcacgccgaagaagtcgctaaatgtgctaagaaaaatgtgcttgaatagctcaaaagtaattagcgtctccattgaaaatccgttatttttaagtgatctagtgttaactatgaatcccaaataaaaagcaaaatccgtaaatgccaaattttcctttttgacgtttttctactgtcgcgagatttgcaagtgtacgtacacttacgatgaattgacagaatctcagctgcgctgatcgtcaattttgtttgggggcacgcccccaatccccctgttattttgaagggaggtgagtcccccttcaaaatcaaaatttaaacagcatctgccgccatcttttcgctgaccttctcacgatgttacacgtggtgttgacacccacttgcatttagagtttcattcaagttgaacattgtgtaatatatgagttgcatttgataaacatatcagttgctatttgtgcaactttaaagcttcggctaattcaacgttctgttaatttacaagcatctcgacagtttctgttaaagcaacatctacgcttcaattcgagcaactcactatacgtatgccgagttgcagacaagctactatatagctgtacgcgctgaaacaccaaaaatcgttcgtttatgcccaataagcgaataatcttgctcaggtgtagtaaaaaactgtttacgtgtagtgaatggcgctagcccttgtcgtaactggcatcatccacgtgtagtaaaacgcgttttactacacgttcgtaattttttcacgtggagtaaatggcgttttactacaccttttgaccccaacgtgctatcacgacaaaccaaaccgcactgcggtttaccccaattttggggtcagttttgccttatgctctttcatgattttaggcgcgttccaagcagtctcaaaaagtggtcgatccaggcgagccgatttttgagaaggattggatagcaactcaatttattttgatcttttgcttggagaaaaacgttcacgttttgaccagggccgtcgcaactgttgaccaaaactcgtccggtaacgtgacgctatttaaacgccgcgttggtttgctagacgaccattcatcatcaccattcaggaggtttttgaaatgacaaagcaagacgaaacacaccgggtcatgttcactttgaccgatcaggcgattgcaaaattgaatcagctggtcgcaaaaaagcaacaggaagtgaatcaaaatccggaactggctaagtaccatgtcagcgtgaccaaatcaaatatcattgaggactggttatcaaagcagtgagtttaaaaagcgctaaagggcctgtactagcgtttcttactctggtgggtataattaatgctctctacatcaaaaacg

SEQ ID NO: 16 - DNA, 11007 bp, Lactobacillus paracasei
  source 1..11007: mol_type = genomic DNA
gccacgaacctgtagccgtttggatgaagccatataatactggaccaaccgccgcaaataagtagccgacactttgagcaaaatcaggaatactaatctactttgcccttaaaaaatcttgagatgatccatatcttgttttgccttcattactgtagttggtcataagaagtgccctacattcattagattacttgtctaataattgtagggcacttgggttgagaaaaatgatgttaactaagaatgcaaacgaactaaaatctttgcttgctttttatccttttctaaggattcaattccttctgaaactaattcatttaattcaatcttttttgtaatgacctgtttgaatagtgaacgatgggtatctataatcttaattactcgatcgaagatattggcatatccataagatgttaataaactaccaccttttttaagaagagctctaacatctacaactggtggatgttgaaataatgcaatcacggtaaccttgccaccatttttaagagcctgaatggcaccagtaagtgtgggttgtacaccggcgcaatcaaacgcaatatccactccctgattttccgtgatagtgctgatagcgtgagctaatgacttttgactatcagcacgtattgggtattgaattcctaattcatttgctaaattcaaacgttcctctgacatgtcatttattatgacgtgatgtgcaccagaaatttgtgctattaaggccgtgaacaatccaattggaccagcaccttgaattaaaacatcatctccaggagacactcggcttgccataactgcctgtgcagcaactgaaactggttcaactaaggcccctaaatcaagcggaaagctagctggtaagagatgtgcaaaggtactttttacattgcacttttcagctaagccaccgttagccgaaaatcctaagaatcctgctgattgatcactacctatagcatgttcacaccaattataatgaccagaaagacattccggacattttccacaagcaatcattggttcgactgcaactttatctccaattcttaatttagatacttgttttccaattttagaaatcgtcccagaaaattcatgaccaggaattagcggggcttgcatatgggttagcggatgaggtattgtcgccaaatccataccctctaaatattcatgaatgtcactaccgcaaataccattaaatgcaacctcaatttgaacttcatctggtgcgggatcaggaatatttcttttttcaaagcggatatccttaggaccgtaaataacagctgccttcaccatagtcatagtgcttcgcttccttcatgttcaatatagcacaatcgtatataaaatagtgaatagatttcagtaatgaagttaccatcttgacttaacaaaaacttgctaactgattatatgagaaacttttacttgaaacatttttggtgattaccattaattccctcggacatattttgaaaaaccctatttgatgctgattgcaaattattttatgcgtatttattaagggtttctatgttgaagtatatagcaaacttgttcaagtaactgactttcacgtgggctttagccaagagatgctgagcagcgaacccaaggggtgttactcgcccacgcaaaaaagaaatccaattgcattccagtatgagcgagaagcaagccattaagacgctgattcatgaactcgcgcacagtgaattacattgtgatccgaagttaaaattggatcgttcaaccatggaattggaagctgaaagtaccgcgtttatcgtttgtcaacatttgggaattgacacgagtgattatacgtttccttaccttgctgtttggtcgaaagataaggatctttcccagctctccaaaagcttaacgcgtatccaatccaccgtcgaaaaattcaataaaaccgtcgatcaaaaccttgaaaagattcgtgagaaaccgttgacgcttgatcaaaaaatagaacgcgctaaaaccattgcgacaacggaaaacatcgcaaaaaaagagcaagggctggtgcaagcaacgcaggagaaaacacgctaacccatttgttgaatactctcactcaagaggacactccagcccttgatcacccaagaaaggaattaccaacatgaaaaccattgacgaaatgaacgaattcgatcgtgacattatcttacttcaccgcaagtctgtgagcgaagatacaccgcaggcaattcttgtgaaagtgaaacagattcgtaacgcaattgccgacgaaaaggcgggtaaagaagatccaattgagaaagaatttacactcgaatgttacgacgaagcaatcagaaaactaagggacctttcggtcgctgattatcagttgtggttgcgtcaaaacaaagacctggaagggtttgaattttgattttgaagggtgtcgtagaccccttcaaaataacgggggattgggggcgtgcccccaaaacaaaattgactatcagcggagctgatattggatcaatttatcgcaagtggacgtccacttgcaaatctcgcgacagtagaaaaagccccagaaaagcaaatctgaaaaaatgtaacaggcacttgatatcaagtgccttattgtttctaggatcgctaaaaataacaggaggtggttacatgaagcaatctgatgaacaccgcacgcgttcagtgagaagcactgtgcgtatgaccccagaggagcgtgcttgggttgatatgagaagagcctctgtcggcaatccaaagttcaatgcatttgcctgtcgcgcactcacgacgagcaagatcgttcatgtacattttactgatactaaaaagttacttagacagctgtcaagaattgggaataaggctcctatgctgtaattacggacaaaaatagtttgtgcgataattacagcataagggcctctaggtcggagcccaggaggcggagaccgccgcacagcccaaccccacgccgaaccggaggccagcccgcccgcaccgcggccgcaatcatccacccaacgccccccaagtttttgatagcggtaacaacgcctgtgcgcttgtcgtggccggccttttttcataaggttggaggagaaaggaagggtggttatgggcgcttggtatgaacacgcaattatttaccaaatctatccaaaatcgtttcaagacagcaacggcgacggcatcggggacctgaacgggatccggcaacggatcccgtacctgcaagccctcggcatcaacacggtgtggctgaacccgatcttcgtctccccacaggtggataacggctacgatgttgccaattacttcgccgtggacgaaaccatgggtacgatggccgacctggaggcgctgatcgcggctctgcacgcggccggcatccgtctgatcatggactttgtgctaaaccacacctcggatcagcacccgtggttccaggacgccattcacgccaaaaatagtctgtaccgcgactactacattttctctggccacgacgggcagctgccaaacaactggggcagcttcttcggcggatcggtttgggcgccggatccggcgggaaccgggcagtcgtattttcatctgttcgaccggcggatgccggatctgaactgggccaatcccgaggtgcggcgggcgatgggagacgtcgccacgttctggctcggcaagggcatcgacggactgcggctggatgccttcatccacattgccaaggccgatctggggcaggattaccccctggctccggggcagcagacgccggtggtggcggagccgtttttctccaacctcccgaaggtgcaggaatggctgcggccgttctgcgaccggatcaaaaccgactaccccgacgcgtttctgctcggcgaggcggcatcggccaacgttaacctggcggcggattacaccgcgcctagccagcacctgatggacagcgtgatcacgttccgctacttcaccgaggacgaaagcggcctggatccgcggctgccggcgcagtaccagccgcggacgctggatttcccggcgttcaagcaaacccaggcggtgtggcagcagaccctcgccggggtgtcgatgccgacgctgtactggggcaaccacgacatggcccggctggcgacgcgggtggccaaaaccaccacccaggcgcgcagtctggccatgctgatgtacctgcagcgcggcctgccggtgatctactatggcgaggagctcgggctacacaacctgcagttcgatcacgttgatcagtttgcggacgtttcggtggcgccgttcgtggccgcggtcgaggccaccgggcagtcgcggagcgcggcgctggccatggtgtcggcgacgcacaaactgccggcacgggggccgatgccttggacgaccgggttgcaccagggcttttccaatcacctgccgtggctggttgggcgcagcgaggacgtgaccagcgtggccgcgcagcaggccgatgaggccagcatgctgcacttctaccaagcgctgattgccctgaagaagcagccgctgtttcaggccgggcattaccggctgctgacgacggcgccgaacctgtacgtctacgaacgcacgctggccagccggcgggccctggtggcggtggccttggatgagcaaggcgccaccttcaccgtgcctgaaggcctgacgaccgtggcgctggccgccggcgattaccaactcgaaggtcaaacgctcacgcttggcgcgaacgccggcgtggtgttaaacgaaaggggaactcgataaccatgcaacttgcagcattacggcaccgcccagaaagcgaagattgttttttgtacactccagatgagctgcggctgcggctccacacagccaaggccgacgtgcaggcggtcatcgtactgtacggggatccgtatgtcaccgcgccgaacccgaccaccggagaaccggaattcgcctaccaagaggcggcgatgatcaaaaccggcaccggccaaaccagcgactactggaccatcagcctgaccgcgccttatcaccgcctgcagtaccagttcctggtgaccggtcaggacggcaacaccgtcctgctcggcgaccgcggcttgcgggccgacagcgccgccaaccgccgggccgatctgttccgggtgccgtacttccacgccatcgacacggtacagacgccggcctgggtcaaggaaaccgtgtggtaccagatattcccggaacgcttcgccaacggggacaagacgaacgaccccaagggcaccaagccttggcgtccggcggatcacccgggccgtgaggattactacggtggcgacttgcaaggggtgctggaccacctggacgacctgcaggcgctcggcgtgaacgggctgtacttctgcccggtgttcacggcgatgtcgaatcacaagtacgacaccatcgactacttcaacatcgaccctgcgtttggcgacaaggccttgttcgccgatctggtcaaccaagcgcaccgccgcggcatgcgggtgatgctggacgctgtgttcaaccacatgggcagccgcagcatgcagtggcaagacgtgctgaagttcggtccgcagtcgcgcttcgcctcctggttccacatcaaccgttttccggcggcgcccttcgccgcgccggaacagggcggcgtgccgcagtacgacaccttcgccttcgaaccgcacatgccgaagctcgacaccagcaacccggcggtgcaggactacctgcttgaggtggcgacgtactggatcaaacagttcgacatcgacgcctggcggctggatgtggccaacgaggtggaccatcacttctggaaacggttcaatcaggcaaccaaagcgctcaagcccgatttcttcgtgctgggcgaggtctggcactccagccagccgtggcttaacggggatgagttcgatggggtcatgaactacgcgttcaccgagcagatcgaggcccacttcctgaccggcaagctgagtgctcctgagctgacggcggcgctgacggatcagctgatgctgtaccgcgaccaaaccgaccaggcgatgctgaacatgctggactcgcatgacaccgcgcggctgctaacggtggccggcggcgacgaggacctggccctgcaggcgctggccttcaccttcctgcaaaccgggatgccgtgcctgtactatggcacggaaatgggcatggccggagaaaacgatcccgactgccggcggccaatggactgggcccagctgaagggcccgatttggcagcgtgtgcaggcccttgtgaccttccgccgcgcccagtcggcaacgctaggcaccggcaccacggcgctgagcgtgaccgcagccgggctgcttaaggtaacccgcacaggtgagcacaccgtgacggcgtattttaacaccaccaagcagatggcgacactgaccgtcagtccattactggcgcagggttacgccggccagcggctggcgccaaccgggtttgctgttatggttcagtaagattatgttagcggtaacaggcaatttgaccttttaaaagcgttttcatattatcataatcaaaagtgtagaaaagttcaggtggcgcaattcacctcccgaaagtgaaggatgcaagatgaaacggatatttgaaatcgacccgtggctggtgcaaagccaccaattgaaccccaacgagaaacgcctgcaggaaagcatgaccgccatcggcaacggctacatgggtctgcgcggtaacttcgaagaaggttacagcggtgatcacctgcaaggcacgtacctcggcggcgtctggttcccagataaaaccgtcgtcggttggtggaaaaacggctacccggattacttcggcaaggcgatcaacgcgccgagcttcatcggcatggcgctcaccgtgaacggcgagcgcgtcgatctggccaccagcgtctaccgcgatttcaccctcacgcttgacctgcaccagggcctgctgacccggagcttcgtgttcgagggcaaaaaggccacggtgcgcttcaccttcaagcgtttcctcagcaacgtaatcaaggaggcggcgctggtgcagctcaccgccgaaagccttgtcggaccggccgagctgacggtggccgcacagctcgacggcaacgtcacgaacgaggacagcaactacgacgagcgcttctgggcaccgcagggggaaaacgccgcggcaggcaccatccagctgcagaccaagcccaacccgttcggggtcccgcagttcacggtgctgctcaagcaaagcctgcgccaaggggcaacccttttacccggcaccgtgaccaccagcaccggccagctgaccagcacggtcacgctgccgctggcgccaaacgtgccggtccagctggaaaaggacgtcatcgtggtcacgagccgcgacgtcgcccctgaggcccaggccgaagcggccgcggagctgatgacacagctgcagggccaaagctttgcggcccagctggcggcacacaccgccctgtgggccaagcgctgggcccaaagcgacgtggtgattgaaggcgacgacgcggcccagcaggggatccgcttcaacctcgcccagctgttcatgacctattacggcgacgataagcggctcaacgtggggccgaagggtttcaccggcgagaagtacggcggggcgacctactgggacaccgaggcgtacgtggtgccgatgtacgtcgccgccacccctccggccgtgacccgggcactgctgcagtaccggcacgaccagctgcccggcgcctaccacaacgcccagcagcaggggctcaaaggggccttgttcccgatggtgaccttcaacggcatcgagtgccacaatgaatgggaaatcaccttcgaggagctgcaccgtaacgcagcggtcgccttcgcgatttaccagtacacggcctacaccggcgatgaaagctacgtcaaccacgacggcatggaggtgctggtgggcatcagccgcttctgggcggaccgggtccacttctccaagcgcgccggcaagtacatgatccacggcgtcaccgggccgaacgagtacgaaaacaacgtcaacaacaactggtacaccaacacgatggccgcctggtgcctggagtacacgctggcccggctgccgaaggccgatgccgccattcaggccaagctggccgtgagcgccgccgagcagcgccagtggcaggacattatcgaccacatgtactatccggaggacaagaagctgggcatcttcgtccagcacgacaccttcctggataaggacctgcggccggcaagctcgattccggccgaccagcggccaatcaaccagcactggtcctgggaccgaatcctgcggtcgccgttcatcaagcaggcggatgtgctccagggcctgtacttcctgaacaatcgcttcacccgcgagcagaaggaacgcaattttgacttctacgagccgctgacggtgcacgaaagctcgctgagtgcctcgattcacgcggtgctggccgccgagctcggtaagcaggataaggccgttgaactctatcagcgtacggctcgtctggacctggacaactacaacaacgatacggcagacggtctgcacatcacctcgatgaccggcggctggctggctatcgtgcagggcttcgccggcatgcgctacgaccacgatcagctgcggttcgatccgttcctgccgaagcagtggcagggttaccagttccgcatcaactaccgcggccgggtgatccaggtcgcggtggggaaaaccgttgcagtgaccctgctggccggcccgccgctgaccgtcatggttgccggccagccgcagcatttggaggtgagcgcgcatgcttaaaggattgctgttcgacctcgacggcgtcttgaccgactcggccaagttccacctgcaggcctggagccagctggccacccagctgggcatcaccctgacgccggccgagcgcgaaggcctgcgcggccgctcgcggctggactcgctgaacctgattttggcggcaggcgcccaggaagaccggttcagtgccgcagagaaaacggcgctaaccgaccagaagaacgcggtgtacctgaagctgattcagacgatgacgccggtggacatcctgccgggcattccgcaactgctgaaggacgcgcaggcggccggcctgaaaatggcaatcgcctcggcgtcgcggaacgccccgacaattcttgaccacctgggcctggccgccagtttcgacgccatcgtcgatccggcgaccctgcaccgcggcaagcccgacccggagatctaccagcaggcgcaagcgctgctggggctccaggccgccgaggtgatcggcttcgaggatgcctcggccggggtcgccgccatcaaagcggccggtcagttcgcggttggcatcggggatgcccggcttctggccgcagcggattacctagtgaaagacacggcggccctgcagctgagccagttgcaagcggcgttcgccaaagaaagtggggagactaatggttgaaatcgacttggaccacctctacaagaagtacgacgacggcgaggattactcggtggtggacttcgaccttcacatcaaggataaggagttcatcgtgttcgtcggcccctcgggctgcgggaagtccaccacgctgcgtatgattgcggggctggaggacattaccaaaggcgagctgaaaatcgacgataaggtgatgaacgacgtggcccccaaggaccgcaacatcgccatggtgtttcagaactacgccttgtacccgcacatgtcagtgtacgacaacatggcgttcggcctaaagctacggcactacaagaaggaggacatcgacaaacgcgtgcaaaacgcggcggagatcctcggcctgaagccgtttctcgaccggaagccggccgccttgtccgggggccagcggcagcgggtggccttgggccgggccatcgtccgcgacgccccaattttcctgatggatgagccgttgtcgaacctggacgcgaagctgcgggtgtccatgcgggcggaaatcgccaagctccaccagcgcctgaacaccaccacgatttacgtgacccacgaccaaaccgaggccatgactatggccgaccgggttgtcgtcatgtccgttggccacgtgcagcagattggcaccccggccgagatttaccagaacccgcggaaccagttcgtggccgggttcatcgggtcgccggcgatgaacttcttcaacatgacctaccaggacggcttcgtcagcgacggccaaagcattcgcctcaaagtgccggaaggccgggcgaagattctggacgaccaagggtacaacggcaaggaagtcgtgttcggcatccggccggaggacatccattcggaggaggccttcctggagacctggccggacgcggttatcagctcaaccgtgtcggtgtcagagctcctgggcgccaccgagcagctttacctgaaggcggatgacaccgagtacgttgccaacgtcaacgcgcgcgacttccacaatcccggggatcatgtgaaaatgggcttcgacgtcaacaaggcgcacttcttcaacaaggacacgaccatggccatcgtggctaagccgattccgctggaaggctgaggaggtgagtgcatgaccccatggtggcagcaagccgtcatttaccagatctacccgaagagttttcaggacagcaacggggatggcatcggcgatttgccggggattaccagtcgccttgattaccttaagcggctgggcgtcgatgccctttggctgagcccagtgtatgtgtcgcccggcgaggacaacggctacgacatcgcggactacgaggccatcgatccccagttcgggacgatggccgacatggacgccttgatcgccgccgccaagcagcgcg

SEQ ID NO: 17 - DNA, 977 bp, Lactobacillus paracasei
  source 1..977: mol_type = genomic DNA
cccgcgattttggcgtgattggcttcgacggggtattcctggaccaggtgtccaaccccaagctgaccacggtgaagcagcccgtgcagcgcctcggcgaactgctggcccgcatgctcctgcagaaggtggcacagtccggcgcccaacagggggagctgctggtcgatcctgagctgattgctcgggacacgacgcgaaagtagatcggatttcaactgtccttaccgctatggtagggccagtttttaggctctatgtcaaatctaattcatagctaatagttgatttggcaacgcctaaggcgtcagccatatcttggtaagtatgatggccttcactgaccagttgagctagcgcaccacgttgaaaacgtgataaagtagaagtacccaaagtaatcactccttatagctggttggaattaactactccattgtaagagattgctttgggcctttttttatttttgttcggattaattatagaatttgtctaattagttgaaaattcttagggttgcccatatatcttttagtctggtcattagtttttatgtttgatctgcttttttctgatcgcaaacacccacaactgcgagtgagtcctttttgaagtcgttgactgtcaacaatacatttatttccacattgacattgacagagccaaagcgcgttgccattagaactgcttccaaaaaagctaataactgtgagtcgcccaaacgtttgattagctaaatcgatacgcttttgcatactaatcctcccgcttgataagaaggtacttaaatagttgctttcaattgatctaatcgccattggcaccatgaaataaaggctaattcgtcaatctttggaatgccataggttctagcatacgttaacttttgagtggtgagtagttgatcatagggtttactaataataccaaccacaaggatatcgacttttttgtcaatcccgttgacaggcttt

SEQ ID NO: 18 - DNA, 1239 bp, Lactobacillus paracasei
  source 1..1239: mol_type = genomic DNA
aaatacgcaaaagaacccgacgagagttaagtctcatcgggttctcagtcgtggatgaattagaagcattgttagctgcataaccttcaacataggatcaatagctggttagatggtcatctctcagactgtttgcaccagatccaggcaaacgtgtttatatccttggtcatactcaggatagatgggcattgtgagtgcaacaggacttagttgcttgtatgctaggcaatgttggccttgatacagaggatcactctgtttctgatctggatgataacctcgtttttccatttcaggacgctgtcccaataattgttgccctgggagtttgtcgaattaagtcttggtgccacaaacacacgctacggcttcttttcttgcaaactgatcttagtatttagggcagttgcatgattacggaattgaaccattttataatgaatcgtcttttctataagtctatagaaagtgcaggtaatggcattttctccagatcggattgtctaatcaatttaattgatttttttggtgtgtttgattatattgcttttgcaaaggtacaatataccttttctctgctgccttgcgagcagcgatggcatcctccatatgaacatatacacgatttaggacaagatggccttgaaagtacagccttgcgacccacttttgagcagttttatcccaactaactccgataacgccagatttgttattggaccgtttaagtgtagaagcaactaaatttgttcgattaattagttgaaaattcttgggattgcccatgtattttttagtctggttattagcttttatgtttgatctgcttatttctgatcgcaaacacccacaactgcgagtgagtcctttttgaagtcgttgactgtcaacgatacatttatttccacattgacattgacagagccaaagcgcgtttccattagaactgcttccaaaaaaactaatgactgtgagtcgtccaaacgtttgattggccaaatcgatacgcttttgcatactaatctccccgtttgataagaaggtacttaaagagttgttttcaattgatctagtcgccattggcaccatgaaataaaggctaattcgtcaatctttggaatgccataggttctagcatacgttaacttttgagtggtaagtagttgatcatagggtttgctaataataccaaccacaaggatatcgacatttttgtcaatcccgttgacaggtttt

(19) United States
(12) Patent Application Publication
(10) Pub. No.: US 2025/0302895 A1
(43) Pub. Date: Oct. 2, 2025

(54) POSTBIOTIC-BASED COMPOSITION FOR THE TREATMENT OF TUMORS

(71) Applicants: POSTBIOTICA S.R.L., Pieve Emanuele (MI) (IT); HUMANITAS MIRASOLE S.P.A., Rozzano (MI) (IT)

(72) Inventors: Giuseppe PENNA, Pieve Emanuele (MI) (IT); Valentina FERRARI, Rozzano (MI) (IT); Maria RESCIGNO, Rozzano (MI) (IT)

(73) Assignees: POSTBIOTICA S.R.L., Pieve Emanuele (MI) (IT); HUMANITAS MIRASOLE S.P.A., Rozzano (MI) (IT)

(21) Appl. No.: 18/863,524

(22) PCT Filed: May 9, 2023
(86) PCT No.: PCT/IB2023/054801

§ 371 Date: Nov. 6, 2024

(30) Foreign Application Priority Data
May 9, 2022 (IT) 102022000009533

(51) Int. Cl. A61K 35/747; A61K 39/00; A61P 35/00; C07K 16/28

(52) U.S. Cl. CPC A61K 35/747; A61P 35/00; C07K 16/2818; A61K 2039/505

(57) ABSTRACT

Methods of treatment and/or prevention of tumours, preferably of solid tumours, more preferably of breast cancer, melanoma, bladder cancer, head and neck cancer, Hodgkin's lymphoma, kidney cancer, non-small cell lung cancer using fermented supernatant, or fractions thereof, of the Lactobacillus casei or paracasei species are disclosed. The species is the strain deposited according to the Budapest Treaty with No. CNCM I-5220 and/or includes in its DNA genome a DNA sequence essentially identical to one of: SEQ ID No 1 to 5.

### FIELD OF THE INVENTION

The invention refers to a composition containing postbiotics of the Lactobacillus casei or paracasei species, and at least one immune checkpoint inhibitor (ICI), and the use thereof in the prevention or treatment of tumours.

### BACKGROUND OF THE INVENTION

Over the past decade, the link between microorganisms and cancer has been established, with nearly 20% of the global cancer burden caused by microbial agents (Pevsner-Fischer 2016). In recent years, the microbiota has been demonstrated to play a role in the pathogenesis and in the course of malignant diseases. In breast cancer, the antibiotic treatment has been associated with the disease development (Rossini 2006) and microbial dysbiosis in breast cancer tissue may be associated with disease stage (Xuan 2014). The gut microbiota was also implicated in clinical responses to the therapeutic strategies (Pitt 2016, Zitvogel 2016), as an enrichment of the specific bacterial strains was described in the responders versus non-responders to ICI-based therapy in the solid malignancies (Gopalakrishnan 2018, Routy 2018, Matson 2018).

In advanced melanoma, the faecal microbiota of ICI-responders transplanted into non-responders was able to sensitize the non-responders to ICI treatment, overcoming primary resistance to therapy (Davar 2021). In addition, the researchers investigated whether the microbial metabolites can influence the immune responses in the context of cancer. Although the published data to date are contradictory, histone deacetylase inhibitors (HDACi), which are commonly secreted by bacteria, have shown potential in sensitizing the tumour cells to the ICIs in vitro (Terranova-Barberio 2017) and are currently being studied in clinical trials in patients with various types of cancer (Jenke 2021).

These results suggest that the gut microbiota and their by-products may influence the immune responses to cancer; however, the molecular mechanism(s) underlying this response must still be elucidated.

Convincing evidence indicates that clinical responses to ICI-based therapy reflect the elimination of the tumour cells by “cognate” CTLs (Pardoll Nat. Rev Canc. 2012; Wei Cancer Discovery 2018). CTL recognition of the tumour cells is mediated by the tumour antigen (TA)-derived peptides that are presented by the HLA class I antigens.

The generation of HLA class I-TA-derived peptide complexes requires a fully functional antigen processing machinery (APM) that generates TA peptides, loads them onto class I HLA alleles, and transports the resulting complexes to the tumour cells membrane.

Abnormalities in the expression and/or in the function of HLA class I APM components, which have been described with high frequency in all cancer types tested, provide tumour cells with an immune escape mechanism due to the defective presentation of the tumour-derived peptides to TA-specific CTLs. Taken together, this basic information provides the rationale for the hypothesis that the association between microbiota and clinical response to ICI-based therapy reflects the ability of the microbial metabolites to increase TA-specific CTL recognition of the tumour cells through upregulation of the expression and/or function of their MHC APM class I component.

The microbiota is formed in the early years of life and several environmental factors contribute to its development, including nutrition (Bokulich, N. A., Chung, J., Battaglia, T., Henderson, N., Jay, M., Li, H., A, D. L., Wu, F., Perez-Perez, G. I., Chen, Y., et al. (2016). Antibiotics, birth mode, and diet shape microbiome maturation during early life. Sci Transl Med 8, 343ra382). Infants and particularly premature infants are susceptible to infections because their immune system is not yet fully developed and functional (Goenka, A., and Kollmann, T. R. (2015). Development of immunity in early life. J Infect 71 Suppl 1, S112-120) (Shane, A. L., Sanchez, P. J., and Stoll, B. J. (2017). Neonatal sepsis. Lancet 390, 1770-1780). The gut microbiota has several effects on the physiological functions of the host, in particular in the development and in the activity of the immune system, favouring, under physiological conditions, tolerance towards commensal bacteria communities, while maintaining, however, the ability to respond to infections by pathogenic bacteria. The molecular mechanisms underlying the host-microbiota interactions depend primarily on a variety of small-sized bioactive molecules derived from the bacterial metabolism and released during the fermentation processes. These metabolites are called postbiotics.

L. paracasei CNCM I-1390 strain has been demonstrated to be able to modulate the inflammatory response of the immune cells through the action of postbiotics that are released (Mileti, E., Matteoli, G., Iliev, I. D., and Rescigno, M. (2009). Comparison of the immunomodulatory properties of three probiotic strains of Lactobacilli using complex culture systems: prediction for in vivo efficacy. PLoS One 4, e7056.). The alleged therapeutic use of the strain of Lacotabacillus paracasei CNCM 1-1390 (deposited according to the Budapest Treaty), redeposited on Jul. 26, 2017 by IEO—Istituto Europeo di Oncologia S.r.I., via Filodrammatici 10, 20121 Milan, Italy, according to the Budapest Treaty at the CNCM (Collection Nationale de Cultures de Microorganismes, Institut Pasteur, 25,28 rue du Docteur Roux 75724 Paris CEDEX 15, FR), with No. CNCM 1-5220 (hereinafter also referred to as B21060), in particular of the fermentation supernatant thereof as anti-inflammatory in the intestinal pathologies is described in application WO2011/009848 A2, incorporated herein by reference.

Methods of fermentation of CNCM strain 1-5220 and uses of this strain are also described in International Applications WO2018024833, WO2019149941 and WO2019149940, incorporated herein by reference.

Postbiotics turn out to be very safe also on inflamed tissues, presumably because they lack molecular motifs associated with the microbes that can, instead, further activate tissue inflammation (Tsilingiri, K., Barbosa, T., Penna, G., Caprioli, F., Sonzogni, A., Viale, G., and Rescigno, M. (2012). Probiotic and postbiotic activity in health and disease: comparison on a novel polarised ex-vivo organ culture model. Gut 61, 1007-1015).

### Prebiotics

Prebiotics are defined as substrates that are selectively utilised by host microorganisms that confer a health benefit (The International Scientific Association for Probiotics and Prebiotics (ISAPP) consensus statement on the definition and scope of prebiotics, Gibson, G. R., et al. (2017). Nat Rev Gastroenterol Hepatol 14, 491-502).

Prebiotic fibres comprise a broad spectrum of nutritional supplements that our body is unable to digest. Instead, prebiotics act as a substrate to promote the growth and the biological activity of particular microorganisms such as bifidobacteria and lactic acid bacteria, bringing many beneficial effects on digestion and overall health. The food prebiotics mostly documented in the literature are the oligosaccharides derived from the non-digestible fructans (fructooligosaccharides (FOS) and inulin) and the galactans (galactolysaccharides or GOS) and their main effects include the activation of the human immune system (Fernandes, R., do Rosario, V. A., Mocellin, M. C., Kuntz, M. G. F., and Trindade, E. (2017). Effects of inulin-type fructans, galactooligosaccharides and related synbiotics on inflammatory markers in adult patients with overweight or obesity: A systematic review. Clin Nutr 36, 1197-1206) and the maintenance of the intestinal homeostasis (Dahiya, D. K., Renuka, Puniya, M., Shandilya, U. K., Dhewa, T., Kumar, N., Kumar, S., Puniya, A. K., and Shukla, P. (2017). Gut Microbiota Modulation and Its Relationship with Obesity Using Prebiotic Fibers and Probiotics: A Review. Front Microbiol 8, 563).

### Probiotics and Postbiotics

Probiotics are defined as viable microorganisms that exert beneficial effects on the host when administered in adequate amounts. Probiotics are generally isolated from stool samples from normal individuals, mostly from breastfed infants. The microbiota may belong to both symbiont and pathobiont classes of microorganisms and may have divergent immunomodulatory properties.

As has been demonstrated in numerous studies, it should be noted that even among the same species, different strains can have opposite effects, (Kaci et al., 2011; Van Hemert et al., 2010). In addition, recent data suggest that certain beneficial effects observed after administration of probiotics could be mediated by molecules or factors produced and secreted by bacteria in the intestinal lumen, hereinafter called postbiotics. In the context of the present invention by postbiotic (or also referred to herein as “fermented supernatant” or “fermented product”) is meant any factor resulting from the metabolic activity of a probiotic or any released metabolic product or molecule, capable of conferring beneficial effects on the host directly or indirectly.

Preferably said postbiotic does not comprise the probiotic.

Recent data have demonstrated that the gut microbiota has an important impact on the clinical response to the immune checkpoint inhibitors (ICIs) in the context of solid tumours. Because the ICI-based therapy acts by unlocking the related CTL effector responses, it is possible that an increased sensitivity to ICI may be due to an improvement in patients' tumour antigen (TA)-specific cytotoxic T lymphocyte (CTL) responses. Clearance of cancer by TA-specific CTLs requires the expression of relevant TAs on class I HLA molecules on the surface of the tumour cells; the reduced expression of HLA of class I is a common mechanism used by the tumour cells to evade the immune system, as it hinders the ability of TA-specific CTLs to recognize and eliminate the tumour cells.

There is still a need for a composition capable of treating tumours.

### DESCRIPTION OF THE INVENTION

The present inventors have found that in vitro treatment with postbiotics increases the HLA class I expression on the surface of the tumour cells, which subsequently increases the TA-specific CTL responses. In addition, the postbiotics combined with anti-PD-1 ICI in vivo increased TA-specific CTL responses and subsequently abrogated tumour growth and prolonged the survival in murine 4T1 triple-negative breast tumour models. These data support a role for the postbiotics in sensitizing the tumour cells to ICI treatment by upregulating the HLA class I expression.

The present inventors have therefore now found and herein shown that microbial-based products, i.e. postbiotics, can sensitize the tumour cells to the lysis of TA-specific CTLs by upregulating the HLA class I expression on the tumour cells in vitro. In addition, if combined with in vivo anti-PD-1 mAb treatment, the postbiotics significantly control tumour growth, prolong survival, and upregulate MHC class I antigen expression in the 4T1 murine breast cancer model, in the CRC CT26 murine model, and in the xenogeneic model of NOD scid gamma (NSG) mice injected subcutaneously with human melanoma cells, supporting the relevance of the postbiotics in the clinic.

The inventors also treated the mice bearing 4T1 with the postbiotic administered via oral gavage instead of i.p. (intraperitoneal) injection, for a more translational approach, and verified that the oral treatment also leads to a control of tumour growth. Also shown herein is that an oral formulation comprising the postbiotic and mannitol provides better results in controlling tumour growth than a formulation comprising maltodextrins.

Since HLA class I expression on the tumour cells is fundamental to the efficacy of ACT (adoptive cell transfer), the inventors sought to understand whether the postbiotic of the invention could control tumour growth even in this system and found that the postbiotic of the invention, combined with ACT (adoptive cell transfer), controls the xenogeneic tumour growth in NOD scid gamma (NSG) mice. In fact, a greater control over tumour growth was found when the mice were pre-treated with the postbiotic compared to the group that received the control carrier, and the tumours weighed significantly less at the end of the experiment than the control. Importantly, the tumour growth was similar among all the treatment conditions prior to ACT, indicating that the postbiotic does not directly act on the tumour growth, blocking or slowing growth.

The present inventors have also surprisingly found that the postbiotic of the invention, in addition to upregulating the HLA class I locus, also upregulates the innate inflammatory pathways, including the toll-like receptor (TLR) signalling cascade and NF-κB. Furthermore, the postbiotic-dependent upregulation of HLA class I is dose-dependent and transient. The postbiotic does not influence instead the HLA class I expression in immune cells; in fact the present inventors have found that the postbiotic of the invention has little or no effect on the HLA class I expression on T cells and on the monocytes. These results demonstrate that the treatment with the postbiotic is safe because it is dose dependent and transient and, through the increased HLA class I expression, “unmasks” the tumour cells, making it “visible” to the immune system resulting in a therapeutic activity potentiation of the cancer immunotherapy. Furthermore, the inventors found that the increase in HLA class I expression, normally induced by IFN-γ, on the tumour cells is dependent on the increase in NLRC5 mediated by the MYD88-NF-κB signal cascade, while the JAK-STAT1 signal pathway (IFN-γ-dependent signal cascade) is not involved. These results support, as a function of its safety profile, an alternative to the non-toxicity-free treatment with IFN-γ, in addition to the use of the postbiotic also in patients refractory to IFN-γ.

The present invention therefore refers in particular to the use of the postbiotic derived from the fermentation of the strain Lactobacillus Paracasei CNCM 1-5220, to be used for the prevention and/or treatment of tumours, in particular said postbiotic is used in combination with at least one inhibitor of the immune checkpoints.

It is therefore an object of the present invention a fermented supernatant, or fractions thereof, of the Lactobacillus casei or paracasei species, said species:
being the strain deposited according to the Budapest Treaty with No. CNCM 1-5220 and/or
being characterized by comprising in their DNA genome at least one DNA sequence essentially identical to one of the sequences selected from the group consisting of: SEQ ID No 1 to 5,
for use in the potentiation of an anti-tumour effect of an agent capable of inducing the CD8+ effector cells, said agent preferably being an immune checkpoint inhibitor (ICI) and/or
for use in activating tumour immunity and/or
for use in a method of treatment and/or prevention of tumours, preferably of solid tumours, more preferably of breast cancer, melanoma, bladder cancer, head and neck cancer, Hodgkin's lymphoma, kidney cancer, non-small cell lung cancer.

It is an object of the present invention a fermented supernatant, or fractions thereof, of the Lactobacillus casei or paracasei species, said species:
being the strain deposited according to the Budapest Treaty with No. CNCM 1-5220 and/or
being characterized by comprising in their DNA genome at least one DNA sequence essentially identical to one of the sequences selected from the group consisting of: SEQ ID No 1 to 5,
for use in the treatment and/or prevention of tumours, preferably of solid tumours, more preferably of breast cancer, melanoma, bladder cancer, head and neck cancer, Hodgkin's lymphoma, kidney cancer, non-small cell lung cancer.

Preferably, the fermented supernatant, or fractions thereof, is for use in the potentiation of an anti-tumour effect of an agent capable of inducing the CD8+ effector cells, said agent preferably being an immune checkpoint inhibitor (ICI) and/or is for use in activating tumour immunity.

Another object of the invention is a fermented supernatant, or fractions thereof, of the Lactobacillus casei or paracasei species, said species:
being the strain deposited according to the Budapest Treaty with No. CNCM 1-5220 and/or
being characterized by comprising in their DNA genome at least one DNA sequence essentially identical to one of the sequences selected from the group consisting of: SEQ ID No 1 to 5,
for use in the potentiation of an anti-tumour effect of an agent capable of inducing the CD8+ effector cells, said agent preferably being an immune checkpoint inhibitor (ICI).

A further object of the invention is a fermented supernatant, or fractions thereof, of the Lactobacillus casei or paracasei species, said species:
being the strain deposited according to the Budapest Treaty with No. CNCM 1-5220 and/or
being characterized by comprising in their DNA genome at least one DNA sequence essentially identical to one of the sequences selected from the group consisting of: SEQ ID No 1 to 5,
for use in activating tumour immunity.

Preferably the method of treatment and/or prevention or the use according to the invention comprises administering the fermented supernatant or fractions thereof and further comprises administering at least one agent capable of inducing the CD8+ effector cells, said agent preferably being an immune checkpoint inhibitor (ICI).

Said administrations are preferably carried out separately. The fermented supernatant is preferably administered by oral administration.

The fermented supernatant, or fractions thereof, is preferably capable of activating tumour immunity by overexpressing (or “upregulating”) class I HLA on the tumour cells and/or by increasing the response of TA-specific CTLs.

In the context of the present invention, preferably the agent or ICI is one or more selected from the group consisting of: anti-PD-1 antibody, anti-CTLA4 antibody, anti-PD-LI antibody, a PD-1 antagonist and fragments thereof, single-chain antibodies and fusion proteins comprising fragments of the antibody, a CpG-oligonucleotide immunotherapeutic agent, cyclophosphamide, immunotherapy, e.g. vaccination, CAR-T, chemotherapy, radiotherapy and any combination thereof and/or wherein the ICI is an inhibitor of a target selected from the group consisting of: CTLA4, PD-1, PDL-1, TIM-3, or LAG-3. Preferably the fermented supernatant is obtainable through a method comprising two fermentations of said Lactobacillus casei or paracasei species in a minimum solution, preferably at least one of the two fermentations is carried out in a minimum solution added with prebiotic fibres.

Preferably, the fermented supernatant is obtainable through a method comprising two fermentations of said Lactobacillus casei or paracasei species, preferably at least one of the two is carried out in a minimum solution added with prebiotic fibres.

The minimum solution is preferably a solution which does not contain carbon and/or nitrogen sources or micromolar concentration of minerals (e.g. iron, sulphur etc.) and which does not comprise prebiotic fibres, preferably the minimum solution is saline, phosphate buffer, H₂O, a minimum isotonic solution or a minimum hypotonic solution.

Preferably the fermentation is carried out at a temperature of 25-40° C., preferably 32° C.-37° C., more preferably 37° C.

Preferably said prebiotic fibres are selected from the group consisting of: fructooligosaccharides (FOS), non-digestible oligosaccharides (NDOs), resistant starch, pectin, beta-glucan, inulin, lactulose, polydextrose, isomaltooligosaccharides (IMO), xylooligosaccharides (XOS), lactitol, chicory root-derived inulin (FOS), arabinoxylooligosaccharides derived from wheat bran (AXOS), xylooligosaccharides (XOS), mannitol, maltodextrin, raffinose, lactulose, sorbitol, galactooligosaccharides (GOS) and combinations thereof, preferably the prebiotic fibres are fructooligosaccharides (FOS).

Preferably the fermented supernatant is obtainable through a method comprising two fermentations of said Lactobacillus casei or paracasei species in a minimum solution, at least one of which is carried out in a minimum solution added with prebiotic fibres, wherein the prebiotic fibres are fructooligosaccharides (FOS) and the minimum solution is saline.

In the present invention, preferably the fermented supernatant is obtainable through a method comprising two fermentations of said Lactobacillus casei or paracasei species, at least one of which is carried out in a minimum solution added with prebiotic fibres, wherein the prebiotic fibres are fructooligosaccharides (FOS) and the minimum solution is saline.

Preferably the fermented supernatant is obtainable by a method comprising the steps of:
a) growing an inoculum of the Lactobacillus strain as defined herein in a suitable culture medium, at a temperature ranging from 4 to 40° C., preferably of 37° C., to obtain a biomass and allowing the fermentation of said biomass in a minimum solution to proceed for 12 to 36 hours, preferably for about 24 hours, to obtain a fermented biomass;
b) centrifuging said fermented biomass to obtain a fermented biomass pellet and a first fermented product;
c) incubating said fermented biomass pellet in a minimum solution and allowing a further fermentation for 12 to 36 hours, preferably for about 24 hours, at a temperature ranging from 4 to 40° C., preferably of 37° C., to obtain a further fermented biomass;
d) separating said further fermented biomass from a second fermented product by centrifugation
   wherein at least one of the minimum solutions of step a) and c) is added with prebiotic fibres, preferably only the minimum solution of step c) comprises prebiotic fibres.

Preferably the fermented supernatant is obtainable by a method comprising the steps of:
a) growing an inoculum of the Lactobacillus strain as defined herein in a suitable culture medium, at a temperature ranging from 4 to 40° C., preferably of 32°-37° or 37° C., to obtain a biomass and allowing the fermentation of said biomass to proceed for 12 to 36 hours, preferably for about 16-24 hours or 24 hours, to obtain a fermented biomass;
b) centrifuging said fermented biomass to obtain a fermented biomass pellet and a first fermented product;
c) incubating said fermented biomass pellet in a minimum solution and allowing a further fermentation for 12 to 36 hours, preferably for about 24 hours, at a temperature ranging from 4 to 40° C., preferably of 37° C., to obtain a further fermented biomass;
d) separating said further fermented biomass from a second fermented product by centrifugation
where the minimum solution is added with prebiotic fibres.

Preferably the supernatant obtained by the methods described above is heated to 70-100° C., preferably at 90° C., preferably for 10 minutes, and then optionally subjected to spray drying or freeze-drying.

Preferably the fermented supernatant is obtainable by a method comprising the steps of:
a) growing an inoculum of the Lactobacillus strain as defined herein in a suitable culture medium, at a temperature ranging from 4 to 40° C., preferably of 37° C., to obtain a biomass and allowing the fermentation of said biomass in a minimum solution to proceed for 12 to 36 hours, preferably for about 24 hours, to obtain a fermented biomass;
b) centrifuging said fermented biomass to obtain a fermented biomass pellet and a first fermented product;
c) incubating said fermented biomass pellet in a minimum solution and allowing a further fermentation for 12 to 36 hours, preferably for about 24 hours, at a temperature ranging from 4 to 20° C., preferably of 4° C., to obtain a further fermented biomass;
d) separating said further fermented biomass from a second fermented product by centrifugation.

Preferably the fermented supernatant is obtainable by a method comprising the steps of:
a) growing an inoculum of the Lactobacillus strain as defined herein in a suitable culture medium, at a temperature ranging from 4 to 40° C., preferably of 37° C., to obtain a biomass and allowing the fermentation of said biomass to proceed for 12 to 36 hours, preferably for about 24 hours, to obtain a fermented biomass;
b) centrifuging said fermented biomass to obtain a fermented biomass pellet and a first fermented product;
c) incubating said fermented biomass pellet in a minimum solution and allowing a further fermentation for 12 to 36 hours, preferably for about 24 hours, at a temperature ranging from 4 to 20° C., preferably of 4° C., to obtain a further fermented biomass;
d) separating said further fermented biomass from a second fermented product by centrifugation.

The minimum solution, preferably the minimum solution of step c), may be added with a lactate salt, preferably sodium lactate, calcium lactate, potassium lactate.

The present invention further comprises any postbiotic derived from the fermentation of different prebiotic fibres by means of the strain Lactobacillus paracasei CNCM I-5220.

Preferably the ICI is an anti-PD-1 antibody.

Preferably, the method of treatment and/or prevention or use according to the invention comprises administering the fermented supernatant or fractions thereof and further comprises administering at least one agent capable of inducing the CD8+ effector cells, said agent being an immune checkpoint inhibitor (ICI), wherein the ICI is an anti-PD-1 antibody.

Preferably the Lactobacillus species is Lactobacillus paracasei, preferably Lactobacillus paracasei is a strain characterized by comprising in its DNA genome at least one DNA sequence essentially identical to SEQ ID No 6 to 18. Preferably said strain comprises in its DNA genome the DNA sequences essentially identical to SEQ ID No 6 to 18.

In the context of the present invention, the supernatant, or fractions thereof is comprised in a pharmaceutical formulation or composition further comprising mannitol, preferably D-mannitol, or a pharmaceutical formulation thereof, preferably at 1-30% (wN), more preferably about at 5% w/V, and wherein preferably said composition or pharmaceutical formulation is pulverized, preferably by spray drying or freeze-drying. Another object of the invention relates to a composition comprising:
a) a fermented supernatant, or fractions thereof, as defined herein and
b) at least one agent capable of inducing the CD8+ effector cells as defined herein.

Preferably, the agent capable of inducing the CD8+ effector cells is an immune checkpoint inhibitor (ICI), preferably anti-PD-1 antibody, and/or an immunotherapy, preferably adoptive cell therapy (ACT).

Preferably, the fermented supernatant, or fractions thereof is comprised in a pharmaceutical formulation or composition further comprising mannitol, preferably D-mannitol, or a pharmaceutical formulation thereof, preferably at 1-30% (wN), more preferably about at 5% w/V, and wherein preferably said composition or pharmaceutical formulation is pulverized, preferably by spray drying or freeze-drying.

Preferably, the fermented supernatant, or fractions thereof, and the at least one agent capable of inducing the CD8+ effector cells are administered separately.

The composition or pharmaceutical formulation according to the invention may be used as a medicament, preferably for use in activating tumour immunity and/or
for use in the potentiation of the anti-tumour effect of the agent capable of inducing the CD8+ effector cells and/or
for use in the treatment and/or prevention of tumours, preferably of solid tumours, more preferably of breast cancer, melanoma, bladder cancer, head and neck cancer, Hodgkin's lymphoma, kidney cancer, non-small cell lung cancer.

The composition or pharmaceutical formulation according to the invention may be used as a medicament, preferably for use in the treatment and/or prevention of tumours, preferably of solid tumours, more preferably of breast cancer, melanoma, bladder cancer, head and neck cancer, Hodgkin's lymphoma, kidney cancer, non-small cell lung cancer,
preferably for use in activating tumour immunity and/or
for use in the potentiation of the anti-tumour effect of the agent capable of inducing the CD8+ effector cells.

The composition or pharmaceutical formulation according to the invention may be used as a medicament,
preferably for use in activating tumour immunity and/or
for use in the potentiation of the anti-tumour effect of the agent capable of inducing the CD8+ effector cells.

A further object of the invention is a composition or pharmaceutical composition or pharmaceutical formulation comprising a fermented supernatant, or fractions thereof, as defined herein
for use in combination with an agent capable of inducing the CD8+ effector cells, said agent is preferably an immune checkpoint inhibitor (ICI), preferably anti-PD-1 antibody, and/or an immunotherapy, preferably adoptive cell therapy (ACT).

A further object of the invention is a fermented supernatant, or fractions thereof, as defined herein to produce an agent for potentiating the anti-tumour effect of an agent capable of inducing the CD8+ effector cells.

A further object of the invention is the use of a fermented supernatant, or fractions thereof, as defined herein, and of an agent capable of inducing the CD8+ effector cells, to produce an agent for activating tumour immunity.

A further object of the invention relates to the use of a fermented supernatant, or fractions thereof, as defined herein, and of an agent capable of inducing the CD8+ effector cells, to produce a medicament.

A further object of the invention is a method for potentiating an anti-tumour effect of an agent capable of inducing the CD8+ effector cells, comprising administering a fermented supernatant, or fractions thereof, as defined herein, to a subject in need thereof.

Another object of the invention is a method for activating tumour immunity, comprising administering a fermented supernatant, or fractions thereof, as defined herein, and an agent capable of inducing the CD8+ effector cells, to a subject in need thereof.

Preferably the composition or formulation or the supernatant of the invention is capable of activating tumour immunity by overexpressing class I HLA on tumour cells and/or by increasing the response of TA-specific CTLs.

Preferably the agent or the ICI is one or more selected from the group consisting of: anti-PD-1 antibody, anti-CTLA4 antibody, anti-PD-LI antibody, a PD-1 antagonist and fragments thereof, single-chain antibodies and fusion proteins comprising fragments of the antibody, a CpG-oligonucleotide immunotherapy agent, cyclophosphamide, immunotherapy, e.g. vaccination, CAR-T or CAR-NK, chemotherapy, radiotherapy and any combination thereof.

In the present invention said fermented supernatant is preferably obtainable by fermentation of said Lactobacillus casei or paracasei species in a minimum solution comprising prebiotic fibres.

Said species are preferably characterized by comprising in their DNA genome the DNA sequences essentially identical to SEQ ID No 1-5.

Preferably, the Lactobacillus species is Lactobacillus paracasei.

More preferably, Lactobacillus paracasei is a strain characterized by comprising in its DNA genome at least one DNA sequence essentially identical to the sequences SEQ ID No 6 to 18, preferably wherein said strain comprises in its DNA genome the DNA sequences essentially identical to SEQ ID No 6 to 18.

In a preferred embodiment of the invention, Lactobacillus paracasei is the strain deposited according to the Budapest Treaty with No. CNCM 1-5220.

In the context of the present invention, the Lactobacillus paracasei species is preferably defined as the strain deposited according to the Budapest Treaty with No. CNCM 1-5220.

In the context of the present invention, the Lactobacillus paracasei species is preferably the strain deposited according to the Budapest Treaty with No. CNCM I-5220.

Preferably said fermented supernatant is obtainable through a method comprising two fermentations of the Lactobacillus casei or paracasei species described herein in a minimum solution, preferably at least one of the two fermentations is carried out in a minimum solution added with prebiotic fibres, the minimum solution being preferably a solution which does not contain carbon and/or nitrogen sources or micromolar concentration of minerals and which does not comprise prebiotic fibres, preferably the minimum solution is saline, phosphate buffer, H₂O, a minimum isotonic solution or a minimum hypotonic solution.

Preferably said fermented supernatant is obtainable through a method comprising two fermentations of the Lactobacillus casei or paracasei species described herein, preferably at least one of the two fermentations, more preferably the second, is carried out in a minimum solution added with prebiotic fibres,
the minimum solution being preferably a solution which does not contain carbon and/or nitrogen sources or micromolar concentration of minerals and which does not comprise prebiotic fibres, preferably the minimum solution is saline, phosphate buffer, H₂O, a minimum isotonic solution or a minimum hypotonic solution.

Preferably the fermentation is carried out at a temperature of 25-40° C., preferably 37° C. or 32-37° C.

Preferably said prebiotic fibres are selected from the group consisting of: fructooligosaccharides (FOS), non-digestible oligosaccharides (NDOs), resistant starch, pectin, beta-glucan, inulin, lactulose, polydextrose, isomaltooligosaccharides (IMO), xylooligosaccharides (XOS), lactitol, chicory root-derived inulin (FOS), arabinoxylooligosaccharides derived from wheat bran (AXOS), xylooligosaccharides (XOS), mannitol, maltodextrin, raffinose, lactulose, sorbitol, galactooligosaccharides (GOS) and combinations thereof, preferably the prebiotic fibres are fructooligosaccharides (FOS).

Preferably the method for obtaining the postbiotic comprises two fermentations of said Lactobacillus casei or paracasei species in a minimum solution, at least one of which is carried out in a minimum solution added with prebiotic fibres, wherein the prebiotic fibres are fructooligosaccharides (FOS) and the minimum solution is saline.

The ICI is preferably an anti-PD-1 antibody.

Preferably the method for obtaining the postbiotic comprises two fermentations of said Lactobacillus casei or paracasei species, at least one of which is carried out in a minimum solution added with prebiotic fibres, wherein the prebiotic fibres are fructooligosaccharides (FOS) and the minimum solution is saline.

The ICI is preferably an anti-PD-1 antibody.

Preferably the fermented supernatant is obtainable by a method characterized by:
a) growing an inoculum of the Lactobacillus strain as defined herein in a suitable culture medium, at a temperature ranging from 4 to 40° C., preferably of 37° C., to obtain a biomass and allowing the fermentation of said biomass in a minimum solution to proceed for 12 to 36 hours, preferably for about 24 hours, to obtain a fermented biomass;
b) centrifuging said fermented biomass to obtain a fermented biomass pellet and a first fermented product;
c) incubating said fermented biomass pellet in a minimum solution and allowing a further fermentation for 12 to 36 hours, preferably for about 24 hours, at a temperature ranging from 4 to 40° C., preferably of 37° C., to obtain a further fermented biomass;
d) separating said further fermented biomass from a second fermented product by centrifugation
wherein at least one of the minimum solutions of step a) and c) is added with prebiotic fibres, preferably only the minimum solution of step c) comprises prebiotic fibres.

Preferably the fermented supernatant is obtainable by a method characterized by:
a) growing an inoculum of the Lactobacillus strain as defined herein in a suitable culture medium, at a temperature ranging from 4 to 40° C., preferably of 37° C., to obtain a biomass and allowing the fermentation of said biomass to proceed for 12 to 36 hours, preferably for about 24 hours, to obtain a fermented biomass;
b) centrifuging said fermented biomass to obtain a fermented biomass pellet and a first fermented product;
c) incubating said fermented biomass pellet in a minimum solution and allowing a further fermentation for 12 to 36 hours, preferably for about 24 hours, at a temperature ranging from 4 to 40° C., preferably of 37° C., to obtain a further fermented biomass;
d) separating said further fermented biomass from a second fermented product by centrifugation
wherein at least the minimum solution of step c) is added with prebiotic fibres.

Preferably, the Lactobacillus species is Lactobacillus paracasei, preferably Lactobacillus paracasei is a strain characterized by comprising in its DNA genome at least one DNA sequence essentially identical to SEQ ID No 6 to 18, preferably wherein said strain comprises in its DNA genome the DNA sequences essentially identical to SEQ ID No 6 to 18.

In the present invention, the prebiotic fibres are preferably selected from the group consisting of: fructooligosaccharides (FOS), non-digestible oligosaccharides (NDOs), resistant starch, pectin, beta-glucan, inulin, lactulose, polydextrose, isomaltooligosaccharides (IMO), xylooligosaccharides (XOS), lactitol, chicory root-derived inulin (FOS), arabinoxylooligosaccharides derived from wheat bran (AXOS), xylooligosaccharides (XOS), mannitol, maltodextrin, raffinose, lactulose, sorbitol, galactooligosaccharides (GOS) and combinations thereof, preferably the prebiotic fibres are fructooligosaccharides (FOS).

Preferably, the composition or pharmaceutical formulation or the postbiotic according to the invention is in the form of a powder, of a liquid, topical formulation, preferably cosmetic (e.g. shampoo, toothpaste, mouthwash, etc.) or cream, solid, preferably capsules or in powder or ocular, preferably eye drops, said formulation, being preferably suitable for ingestion, for topical application, introduced into an enema for external or internal use. In the composition of the invention, the fermented product, or fractions thereof, is preferably present at 0.02-40% weight/volume (w/v), more preferably 1% weight/volume (w/v).

A further object of the invention is a composition, for example a probiotic, pharmaceutical, nutraceutical, cosmetic, food composition or food supplement or feed based comprising the fermented or postbiotic product, or fractions thereof, as described herein for the uses described herein.

Preferably, the composition of the invention may further optionally further comprise adjuvants and/or therapeutic agents, preferably at least one anti-tumour drug. The composition according to the invention is preferably for use as a medicament.

In a preferred embodiment, both the minimum solution of step a) and that of step c) comprise prebiotic fibres.

The first and/or second fermented product obtainable respectively from step b) or d) of the methods described herein are an object of the invention and altogether can be defined as “fermented product”. They can be used as active ingredients for the composition and the formulation, both individually and in combination. In the case where only step c) includes prebiotic fibres, only the second fermentation product is preferably to be considered as object of the invention.

Within the scope of the present invention “the supernatant in solution” or the “fermented supernatant” may also be referred to as “fermented product” or “postbiotic” or “fermentation product” and may also comprise fractions and/or metabolic components thereof. The fermentation products used in the present invention can be obtained through the fermentation of different substrates such as sodium lactate or prebiotic ingredients (GOS, FOS, etc.), plant extracts (e.g. Aloe, chamomile, mallow, echinacea). The fermented product or the fermented composition or composition or formulation or supernatant according to the invention may be freeze-dried according to any method known to the person skilled in the art or it may be liquid.

In the context of the present invention the terms “fermented product” and “fermented supernatant” are interchangeable.

A further object of the invention is a method of producing the composition or formulation described above, which comprises adding the fermented product defined above, or fraction thereof, to at least one agent capable of inducing the CD8+ effector cells, and optionally to at least one carrier and/or diluent and/or excipient and/or adjuvant and/or therapeutic agent.

Preferably the minimum solution is a saline, phosphate buffer, H2O, etc. It may for example be a minimum isotonic or hypotonic solution. In the context of the present invention a “minimum isotonic solution” is defined as a solution, with osmolarity similar to blood and body fluid (290 mOsmol/1), while a “minimum hypotonic solution” is a solution having an osmolarity lower than that of body fluid (<280 mOsmol/1) capable of actively facilitating the absorption of liquids.

In a preferred aspect of the invention a postbiotic product obtained from the fermentation of the fructooligosaccharide (FOS) from L. paracasei CNCM 1-5220 which does not contain foodstuffs and live bacteria is used. The fermented product or composition according to the invention may be freeze-dried according to any method known to the person skilled in the art. The food product or food supplement according to the invention comprises an amount of the composition (or fermented product) defined above capable of conferring the above-described properties to the food product.

Preferably the fermented supernatant does not comprise prebiotic fibres.

The fermented supernatant as defined herein is preferably in the form of a dry powder, for example obtained by lyophilization, granulation, spray drying or freeze-drying.

The composition or formulation of the invention preferably comprises carriers and/or diluents and/or excipients.

The composition or the formulation of the invention provides for both the postbiotic and the agent capable of inducing the CD8+ effector cells to be administered simultaneously whether they are administered separately and/or at time intervals.

By composition is also meant a combination.

By combination can be meant both simultaneous and separate administration and/or at time intervals of the postbiotic and of the agent capable of inducing the CD8+ effector cells.

A further object of the invention is a composition or pharmaceutical composition or pharmaceutical formulation for use in combination with an immune checkpoint inhibitor, comprising the postbiotic as defined herein.

By use in combination is meant both simultaneous and separate use and/or at intervals of time.

A further object of the invention is the use of the postbiotic as defined herein to produce an agent to potentiate the anti-tumour effect of an immune checkpoint inhibitor.

A further object of the invention is the use of the postbiotic as defined herein and of an immune checkpoint inhibitor, to produce an agent for activating tumour immunity.

A further object of the invention is the use of a combination of a postbiotic as defined herein and an immune checkpoint inhibitor for the production of a medicament.

Other objects of the invention are a method for potentiating an anti-tumour effect of an immune checkpoint inhibitor, comprising administering a postbiotic as defined herein to a subject in need thereof and a method for activating tumour immunity, comprising administering a postbiotic and an immune checkpoint inhibitor to a subject in need thereof.

A further object is the use of the postbiotic as defined herein and of an immune checkpoint inhibitor, for the production of an agent for the activation of tumour immunity or of a medicament.

The present invention also refers to a composition as defined herein further comprising adjuvants and/or therapeutic agents, preferably at least one anti-tumour drug.

The fermented supernatant as defined herein is preferably obtained by a method comprising the steps:
a) culturing an inoculum of Lactobacillus strain as defined herein in a suitable culture medium, at a temperature ranging from 4 to 40° C., preferably of 37° C., to have a biomass and allowing the fermentation of said biomass in a minimum solution to proceed from 12 to 36 hours, preferably for about 24 hours, to obtain a fermented biomass;
b) centrifuging said fermented biomass to obtain a fermented biomass in pellets and a first fermented product;
c) incubating said fermented biomass in pellets in a minimum solution and allowing a further fermentation from 12 to 36 hours, preferably for about 24 hours, at a temperature ranging from 4 to 40° C., preferably of 37° C., to obtain a further fermented biomass;
d) separating said further fermented biomass from a second fermented product by centrifugation
wherein at least one of the minimum solutions of step a) and c) comprises prebiotic fibres, preferably only the minimum solution of step c) comprises prebiotic fibres.

The fermented supernatant as defined herein is preferably obtained by a method comprising the steps:
a) culturing an inoculum of Lactobacillus strain as defined herein in a suitable culture medium, at a temperature ranging from 4 to 40° C., preferably of 37° C., to have a biomass and allowing the fermentation of said biomass to proceed from 12 to 36 hours, preferably for about 24 hours, to obtain a fermented biomass;
b) centrifuging said fermented biomass to obtain a fermented biomass in pellets and a first fermented product;
c) incubating said fermented biomass in pellets in a minimum solution and allowing a further fermentation from 12 to 36 hours, preferably for about 24 hours, at a temperature ranging from 4 to 40° C., preferably of 37° C., to obtain a further fermented biomass;
d) separating said further fermented biomass from a second fermented product by centrifugation
wherein the minimum solution of step c) comprises prebiotic fibres.

Preferably the minimum solution is a solution which does not contain carbon and/or nitrogen sources or micromolar concentration of minerals and which does not comprise prebiotic fibres, preferably the minimum solution is saline, phosphate buffer, H₂O, a minimum isotonic solution or a hypotonic solution.

In the present invention the agent capable of inducing the CD8+ effector cells can be defined as at least one immune checkpoint inhibitor (ICI) or as one or more of the agents selected from the group consisting of: anti-PD-1 antibody, anti-CTLA4 antibody, anti-PD-LI antibody, a PD-1 antagonist and fragments thereof, single-chain antibodies and fusion proteins comprising fragments of the antibody, a CpG-oligonucleotide immunotherapeutic agent, cyclophosphamide, immunotherapy, e.g. vaccination, CAR-T or CAR-NK, chemotherapy, radiotherapy and any combination thereof and/or wherein the ICI is an inhibitor of a target selected from the group consisting of: CTLA4, PD-1, PDL-1, TIM-3, or LAG-3.

In the context of the present invention, the agent capable of inducing the CD8+ effector cells can be defined as at least one costimulatory molecule inhibitors, a dendritic cell therapy, a CAR-T and/or CAR-NK cell therapy, a cytokine therapy or an adoptive cell therapy, for example with adoptive T cells.

In the context of the present invention the immunotherapy preferably comprises checkpoint inhibitors, costimulatory molecule inhibitors, dendritic cell therapy, CAR-T or CAR-NK cell therapy, cytokine therapy or adoptive cell therapy, for example with adoptive T cells.

The chimeric antigen receptors (CAR, also known as chimeric immunoreceptors, chimeric T cell receptors, or artificial T cell receptors) are engineered receptors that combine a new specificity with an immune cell to target the tumour cells. Typically, these receptors graft the specificity of a monoclonal antibody onto a T cell. CAR-T cell therapy refers to a treatment that uses such transformed cells for cancer therapy. The basic principle of CAR-T cell design involves the recombinant receptors that combine the functions of binding the antigen and of activation of the T cells. The general premise of the CAR-T cells is to artificially generate T cells targeted to the markers found on the tumour cells. Scientists can remove the T cells from a person, genetically modify them, and put them back in the patient so they can attack tumour cells. Once the T cell has been engineered to become a CAR-T cell, it acts as a “living drug”. CAR-T cells create a linkage between an extracellular ligand recognition domain and an intracellular signalling molecule that in turn activates the T cells. The extracellular ligand recognition domain is usually a single-chain variable fragment (scFv). An important aspect of the safety of CAR-T cell therapy is how to ensure that only the tumour cells and not the normal cells are targeted. The specificity of the CAR-T cells is determined by the choice of the targeted molecule.

The term adoptive cell therapy (ACT) is interchangeable with adoptive cell transfer (ACT).

The adoptive T cell therapy is a form of passive immunization by T-cell transfusion (adoptive cell transfer). They are found in blood and in the tissues and are usually activated when they encounter foreign pathogens. Specifically, they are activated when the T cell surface receptors encounter cells that show parts of foreign proteins on their surface antigens. These may be infected cells or antigen presenting cells (APC). They are found in normal tissue and in the tumour tissue, where they are known as tumour-infiltrating lymphocytes (TIL). They are activated by the presence of APCs such as the dendritic cells that present tumour antigens. Although these cells can attack the tumour, the environment within the tumour is highly immunosuppressive, preventing immune-mediated tumour death. Several ways have been developed to produce and obtain tumour-targeted T cells. T cells specific for a tumour antigen can be removed from a tumour sample (TIL) or filtered from the blood. The subsequent activation and culture are performed ex vivo, and the resulting cells are then reinfused. Activation can occur through gene therapy or by exposing the T cells to the tumour antigens. A further object of the invention is a composition or pharmaceutical formulation comprising a fermented supernatant, or fractions thereof, as defined herein and mannitol, preferably D-mannitol, or a pharmaceutical formulation thereof, at 1-30% (w/), more preferably about at 5% wN, wherein the composition or pharmaceutical formulation is preferably pulverized, preferably by spray drying or freeze-drying.

Another object of the invention is a method for producing the composition as defined herein, comprising adding the fermented supernatant, or fraction thereof, as defined herein, to at least one immune checkpoint inhibitor.

In a preferred embodiment of the invention, the composition or formulation of the invention comprises a fermented product defined as above and a vitamin selected from the group of vitamin D3, vitamin K, vitamin B1, B6, B12 and/or at least one chemical element such as zinc or galactogogue, and or at least one herbal extract (such as echinacea, mallow, chamomile, etc.).

In a preferred embodiment of the invention, the composition or formulation comprises from 0.4% to 20% of postbiotic added with at least one vitamin selected from the group of vitamin D3, vitamin K, Vitamin B1, B6, B12 and/or at least one chemical element such as Zinc or galactogogue, and/or at least one herbal extract (such as echinacea, mallow, chamomile, etc.).

In a preferred embodiment of the invention, the composition or formulation comprises from 0.4% to 20% postbiotic added with at least one vitamin selected from the group of vitamin D3, vitamin K, Vitamin B1, B6, B12 and at least one chemical element such as Zinc or galactogogue, and at least one herbal extract (such as echinacea, mallow, chamomile, etc.).

Another object of the invention is the use of the fermentation product defined as above for the preparation of a nutraceutical composition or pharmaceutical composition or of a pharmaceutical formulation.

Another object of the invention is a probiotic, pharmaceutical, nutraceutical, food composition, food supplement or feed comprising the composition or the supernatant as defined herein.

The pharmaceutical composition according to the invention is formulated to be administered to an individual in a therapeutically effective amount, depending for example on the type of the subject, the severity of the pathology and the route of administration. Usually, the therapeutically effective amount of the fermented product is about 1-1000 mg/day, preferably 200 mg/day or for example 1 to 1000 mg, preferably 2 g. Administration for example is performed by 1-2 administrations/day of 1-3 g of product, preferably 2 g of fermented product or composition as defined above, at the concentration of 0.1 up to 40% of product.

The fermented product (or composition or pharmaceutical formulation comprising it) may be administered by any suitable route of administration. For example, the composition according to the invention may be administered to animals (including humans) in an ingestible oral form. In the case of a food or nutraceutical composition, the fermented product can simply be incorporated into a conventional foodstuff or food supplement. Examples of pharmaceutical formulations include capsules, microcapsules, tablets, granules, powder, dragées, pills, suppositories, suspensions and syrups, oral gavage. In another embodiment, the composition is in a form for rectal administration in animals (including humans), for example a rectal suppository or enema. Suitable formulations can be prepared by applying commonly used methods using conventional organic and inorganic additives. The amount of active ingredient in the medical composition may be at a level such as to exert the desired therapeutic effect. The composition may contain further useful ingredients, including the probiotics. The formulation may contain structurants (fillers) and extenders, such as maltodextrins or pullulan.

In the present invention, 1, 2, 3, 4, . . . doses of postbiotic may be administered at regular intervals.

In the context of the present invention, when reference is made to specific DNA sequences, it is meant that RNA molecules identical to said polynucleotides are also included in the invention, except for the fact that the RNA sequence contains uracil instead of thymine and the backbone of the RNA molecule contains ribose instead of deoxyribose, RNA sequences complementary to the sequences described therein, functional fragments, mutants and their derivatives, proteins encoded by them, functional fragments, mutants and their derivatives.

The term “complementary” sequence refers to a polynucleotide that is not identical to the sequence but has a base sequence complementary to the first sequence or encodes the same amino acid sequence as the first sequence. A complementary sequence may comprise DNA and RNA polynucleotides.

The term “functional” or “functional” can be understood as being able to maintain the same activity. The “fragments” are preferably at least 10 aa., 20 aa., 30 aa., 40 aa., 50 aa., 60 aa., 70 aa., 80 aa., 90 aa., 100 aa., 150 aa., 200 aa., 300 aa., 400 aa., 500 aa., 600 aa., 700 aa., 800 aa., 900 aa., 1000 aa., 1200 aa., 1400 aa., 1600 aa., 1800 aa. or 2000 aa long. “Derivatives” can be recombinant or synthetic. The term “derivative” as used herein in reference to a protein means a chemically modified protein or an analogue thereof, wherein at least one substituent is not present in the unmodified protein or an analogue thereof, i.e. a protein which is covalently modified. Typical modifications are aminides, carbohydrates, alkyl groups, acyl groups, esters and the like. As used herein, the term “derivatives” also refers to longer or shorter sequences of polynucleotides/proteins and/or having for example, a percentage of identity of at least 85%, 86%, 87%, 88%, 89%, 90%, 91%, 92%, 93%, 94%, 95%, 96%, 97%, 98%, more preferably of at least 99%, with the sequences described herein.

In the present invention, “at least 70% identity” means that the identity can be a sequence identity of at least 70%, or 75%, or 80%, or 85% or 90% or 95% or 100% with respect to the indicated sequences. This applies to all the aforesaid % of identity. Preferably, the % of identity concerns the entire length of the indicated sequence.

In the present invention by “essentially identical” is meant for example a sequence having a percentage of identity of at least 98%, more preferably of at least 99%.

In the context of the present invention a carrier may be a vehicle or composition involved in releasing the fermented product within the subject or facilitating the accumulation of the composition.

The derivative of the invention also includes “functional mutants” of the polypeptides, which are polypeptides that can be generated by mutating one or more amino acids in their sequences and that maintain their activity. Indeed, the polypeptide of the invention, if required, can be modified in vitro and/or in vivo, for example by glycosylation, myristylation, amidation, carboxylation or phosphorylation, and can be obtained, for example, by synthetic or recombinant techniques known in the art. In the present invention “functional” is understood for example as “maintaining its activity” e.g. immunomodulatory activity or anti-inflammatory activity or the activities described herein.

Also within the scope of the present invention there are polynucleotides having the same nucleotide sequences as a polynucleotide exemplified herein except for the nucleotide substitutions, additions or deletions within the polynucleotide sequence, so long as these variant polynucleotides maintain substantially the same relevant functional activity as the polynucleotides specifically exemplified herein (e.g., encode a protein having the same amino acid sequence or functional activity encoded by the exemplified polynucleotide). Thus, the polynucleotides described herein should be understood to comprise mutants, derivatives, variants and fragments, as discussed above, of the specifically exemplified sequences. The invention in question also contemplates those polynucleotide molecules having sequences that are sufficiently homologous with the polynucleotide sequences of the invention so as to allow hybridization with such sequence under standard stringent conditions and standard methods (Maniatis, T. et al, 1982). The polynucleotides described herein may also be defined in terms of more particular intervals of identity and/or similarity to those exemplified herein. The sequence identity will typically be greater than 60%, preferably greater than 75%, more preferably greater than 80%, even more preferably greater than 90% and may be greater than 95%. The identity and/or the similarity of a sequence may be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% or greater with respect to a sequence exemplified herein. Unless otherwise specified, as used herein the percentage of sequence identity and/or similarity of two sequences can be determined using the algorithm of Karlin and Altschul (1990), modified in Karlin and Altschul (1993). This algorithm is incorporated into the NBLAST and XBLAST programs of Altschul et al. (1990). BLAST searches can be performed using the NBLAST program, score=100, word length=12, to obtain the sequences with the desired percentage of sequence identity. For comparing alignments with discontinuities use is made of Gapped BLAST as described in Altschul et al. (1997). When using the BLAST and gapped BLAST programs, the default parameters of the respective programs (NBLAST and XBLAST) can be used. See NCBI/N1H website.

According to the present invention, the above-mentioned prebiotic fibres are contained in the fermentation method in an amount of 0.5-25% by weight or higher, preferably in an amount of 10% or higher or lower. The above-mentioned prebiotic fibres can be added to the minimum solution in an amount of e.g. 5 g/L.

According to the present invention, the pH of the fermentation medium is balanced to be in the interval of 4.5-7.0 or 5-7.5 prior to fermentation. The osmolarity of the postbiotic is preferably 15-30 mOsM/L, preferably presents a clear/slight turbidity and is colourless. It is preferable that the pH is maintained within the above-described interval, by diluting with distilled water, etc., for example, without using pH regulators after appropriately transforming the plant raw material, or appropriately adjusting the type or the amount of plant raw material. If it is necessary to use a pH regulator, one normally used for foodstuffs may be added, as long as the effect of the present invention is not impaired, and the type thereof is not particularly limited. Examples of a preferable acid include citric acid and potassium sorbate, and examples of a preferable base include potassium carbonate. If the pH regulator used is in the form of a crystal, it is preferable to use an aqueous solution thereof.

It is preferable that Lactobacillus casei or paracasei as defined above is used for fermentation of the culture medium after being precultured. Pre-culturing can be carried out by conventional methods.

Fermentation of the culture medium using Lactobacillus casei or paracasei can be carried out by conventional methods. For example, the above-mentioned precultured product may be inoculated to cultivate said Lactobacillus casei or paracasei. The amount of inoculum is preferably between 0.1 and 10% by volume, the temperature during cultivation is between 4 to 40° C., preferably at 32-37° C., more preferably 37° C. and the culture time is preferably 16-24 or 16-30 hours. According to the present invention, the pH of the fermentation product at the end of fermentation is 5.5 or higher and lower than 7.0 or is 4.50-7.0. With the pH in this interval, a valid degree of fermentation can be obtained, to produce a fermented food or beverage with excellent taste, flavour and preservation. The fermented product thus obtained can be directly used as fermented food or beverage. Alternatively, if necessary, appropriate additives may be added, or the product may be appropriately processed to become fermented food or beverage. The composition according to the invention further comprises a fermented product of Lactobacillus casei or paracasei with the same bacteriological properties as those of the CNCM 1-5220 strain.

The fermentation product of the present invention is preferably obtained by fermentation using a CNCM 1-5220 strain, or a mutant strain of the CNCM 1-5220 strain having the same capacity, or a strain isolated from Lactobacillus casei or paracasei having the same bacteriological properties as those of the CNCM 1-5220 strain.

The present invention refers to a fermentation product, obtained by fermentation of a material containing prebiotic fibres (in particular, a material containing FOS), by a specific strain of Lactobacillus casei or paracasei, and more in particular, to a fermentation product having immunomodulatory effects.

Furthermore, the present invention refers to a fermented food having an immunomodulatory effect, which contains the fermentation product. FOS are soluble dietary fibres belonging to the family of fructans. They are composed of linear chains of fructose units, linked by β-glycosidic fructose-fructose bridges (2(11)). The number of fructose units varies from 2 to 60 and often ends with one glucose unit. They can be produced starting from the degradation of inulin or through transfructosylation processes.

In the present invention, the FOS-containing material is not limited as long as it contains FOS, but the material is preferably the one containing high concentrations of FOS. In particular, the material is preferably one containing one or more plants selected from Topinambur, burdock, chicory, onions, asparagus, wheat, beans, tomatoes, fennel and other fruits, such as currants, ursina grapes and vegetables and cereals.

The fermentation product of the present invention is obtained by fermentation of a material containing prebiotic fibres by means of the CNCM 1-5220 strain, or a mutant of the CNCM 1-5220 strain having the same capacity, or a strain isolated from Lactobacillus casei or paracasei having the same bacteriological properties as those of the CNCM 1-5220 strain.

Fermentation may proceed until the FOS contained in the aforesaid material is sufficiently degraded.

In particular, the fermentation takes place under anaerobic conditions at a temperature of 4 up to 40° C., preferably from 10 to 37° C.

It is desirable that the fermentation takes place at pH from 4 to 8.0, preferably from 5.0 to 7.0, particularly preferably about 6.5.

Fermentation can be carried out as long as the prebiotic fibres contained in the aforesaid material are sufficiently degraded, and for example, fermentation can proceed for 16 up to 48 hours.

The fermentation product of the invention induces the HLA class I expression on the tumour cells, increasing the activation of cytotoxic T lymphocytes (CTLs) specific for tumour antigens.

It should be noted that the aforesaid fermentation product is preferably contained at a concentration of 1 to 20% or more in terms of dry weight.

To achieve the immunomodulatory effect, the composition or the postbiotic may be administered once or twice daily at 0.1 up to 2.0 g/dose in terms of dry weight or fermented product content.

The invention will now be illustrated with the aid of non-limiting examples with reference to the following figures.

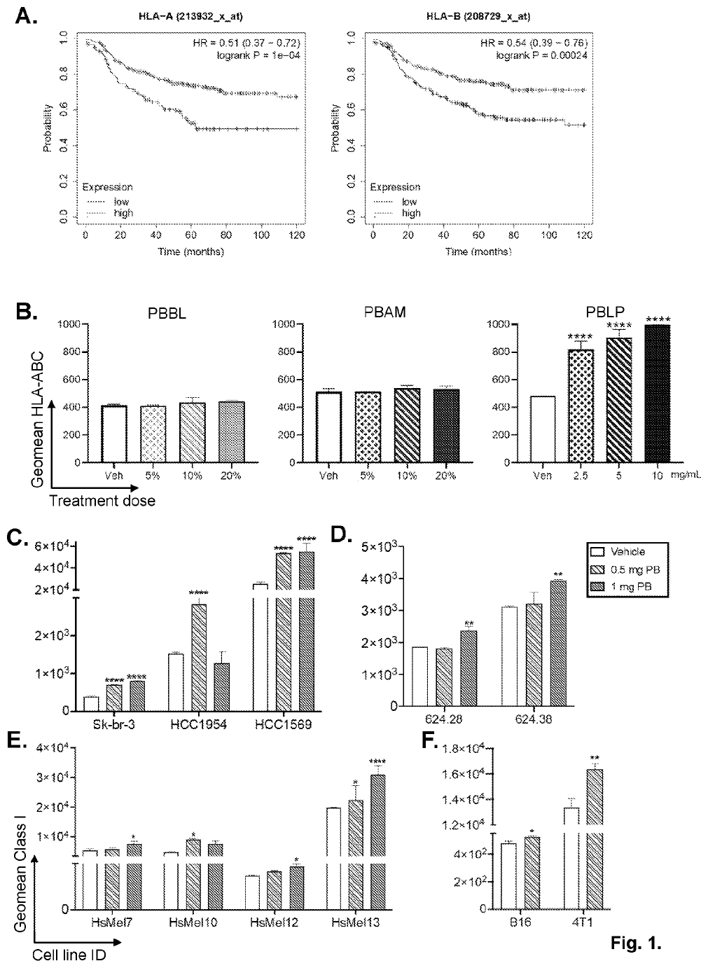

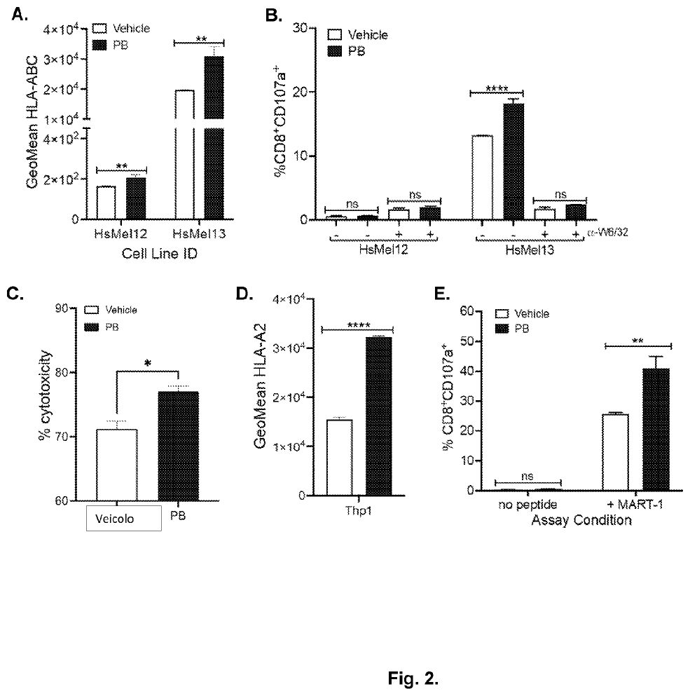

FIG. 1. The in vitro treatment with the postbiotic increases the HLA Class I expression on human melanoma and breast tumour cells lines. A) Analysis of the data obtained from The Cancer Genome Atlas (TCGA) for correlation of HLA-A and HLA-B expression with breast cancer survival. B) The breast tumour cells line SK-BR-3 was incubated with 3 concentrations of postbiotics released from 3 different bacterial strains. The geometric mean of the HLA-ABC expression was measured by flow cytofluorimetry. C) breast D) melanoma E) primary melanoma cells, and F) murine lines of melanoma and breast cancer were treated with 0.5 mg (bars), 1 mg (dark bars) of postbiotic or with the relative carrier control (empty bars). After 48 hours, the geometric mean of the surface expression of HLA Class I (C-E), H-2k^{b}(B16) and H-2k^{d} (4T1) (F) was measured by flow cytofluorimetry. The experiments were replicated at minimum in triplicate. The statistical analysis was performed by means of the 2-way ANOVA method, *p<0.05, **p<0.01, ***p<0.001, ****p<0.0001.

FIG. 2. Postbiotic-induced induction of the HLA class I expression on the tumour cells lines increases the activation of cytotoxic T lymphocytes (CTLs) specific for tumour antigens. A) MART-1 antigen negative (Mel12) or positive (Mel13) human primary cells of melanoma were pre-treated for 48 hours with the postbiotic or the control carrier, the geometric mean of HLA-ABC (clone antibody W6/32) was measured by flow cytometry. B) The melanoma cells pre-treated like (A) were cultured for 5 hours with MART-1²⁶⁻³⁵-specific CTLs, the percentage of CD8⁺CD107⁺ cells was measured by flow cytometry. C) The Thp1 cell line was pre-treated with the postbiotic or the control carrier for 48 hours prior to measurement of the HLA-A2 expression by flow cytometry. D) The Thp1 cells like (C) were cultured for 5 h with MART-1²⁶⁻³⁵-specific CTLs in the presence of MART-1²⁶⁻³⁵-specific peptide, the percentage of CD8⁺CD107⁺ cells was measured by flow cytometry. All experiments were repeated in triplicate. The statistical analysis was performed by means of the 2-way ANOVA method, **p<0.01, * ***p<0.0001.

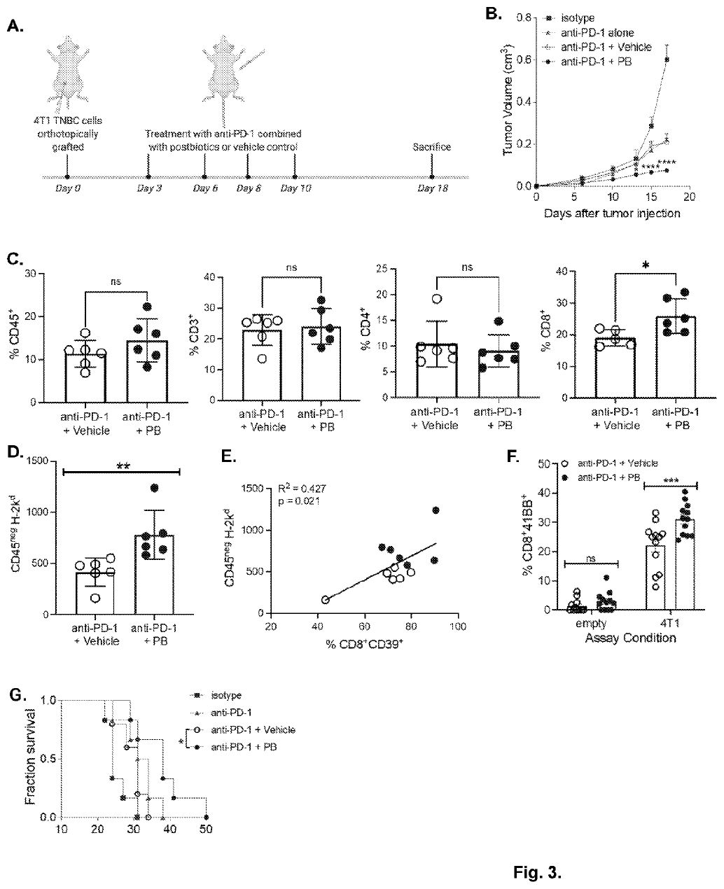

FIG. 3. The in vivo treatment with the postbiotic in combination with anti-PD1 controls tumour growth and improves survival. A-D) 6-8-week-old Balb/c mice were orthotopically injected into the breast fat pad with 1.5×10⁵ 4T1 tumour cells and separated into 4 experimental groups on day 3 based on the tumour equivalent size. The mice were treated with 250 μg of postbiotic (PB) or control carrier in combination with anti-PD1 (200 μg; clone 29F.1A12) from day 3 and continuing every other day for a total of 4 injections. A) Experimental scheme B) The tumour measurements were performed every other day using a calliper and the volume was calculated using the following formula: tumour volume=(length×width²)÷2. C, D) The tumours were labelled and the geometric mean was assessed by flow cytometry. E) CD8⁺CD39⁺ cells in the tumour tissue were correlated with MHC class I expression of the tumour. F) 2×10⁵ splenocytes were co-cultured in 1:1 ratio with 4T1 tumour cells in vitro and T cell activation (percentage of CD8⁺41 BB⁺) was assessed by flow cytometry. G) Survival curve. The experiments were repeated in triplicate and the statistical analysis was carried out by means of the 2-way ANOVA method (*p<0.05, **p<0.01, ***p<0.001, ****p<0.0001).

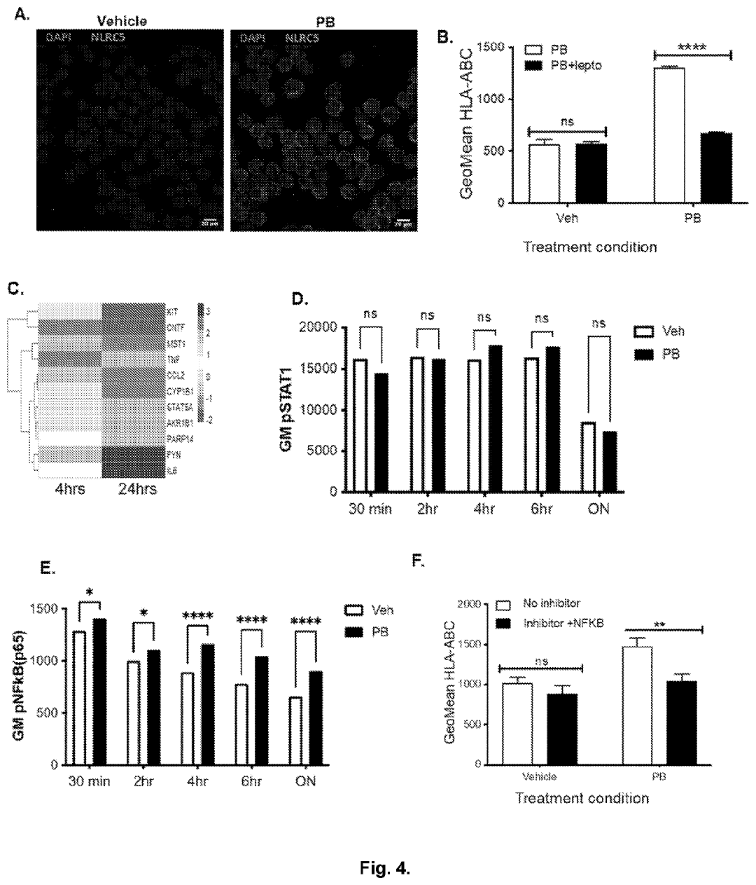

FIG. 4. Mechanism of increased postbiotic-induced HLA class I expression. The SK-BR-3 cell line was incubated with the postbiotic or the control carrier for 48 hours prior to analysis of NLRC5 expression by A) immunofluorescence. B) The SK-BR-3 line was treated with 100 ng/ml of leptomycin B and 1 mg of carrier or postbiotic for 48 hours prior to analysis of HLA-ABC (W6/32) expression by flow cytometry. C) RNA analysis of the SK-BR-3 line after 4 and 24 hours incubation with the postbiotic. The results were normalized towards the carrier and expressed as fold-change (how much it increases) D). Phosphorylation of STAT1 and E) NF-kB was measured by flow cytometry. All experiments were repeated in triplicate. The statistical analysis was carried out by means of the 2-way ANOVA method. F) Treatment with 10 ng/ml of NF-KB inhibitor and 1 mg/ml for 48 hours prior to HLA-ABC analysis (measured by flow cytometry.

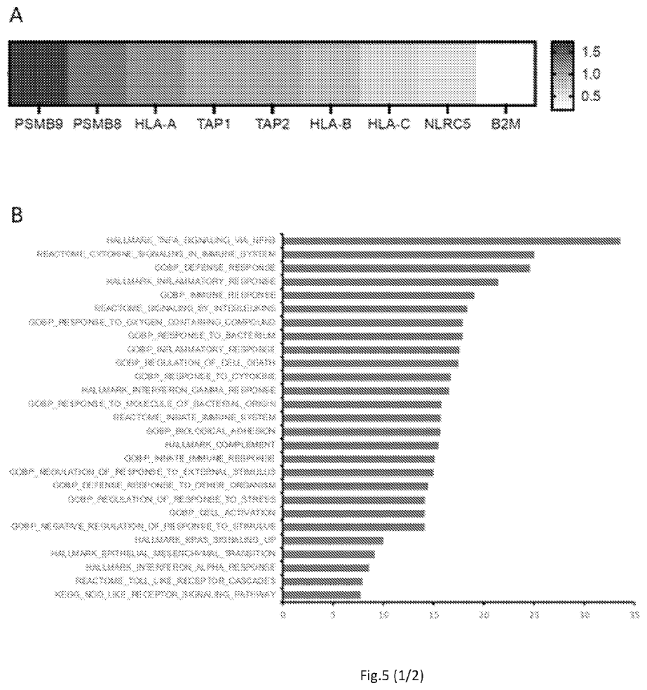

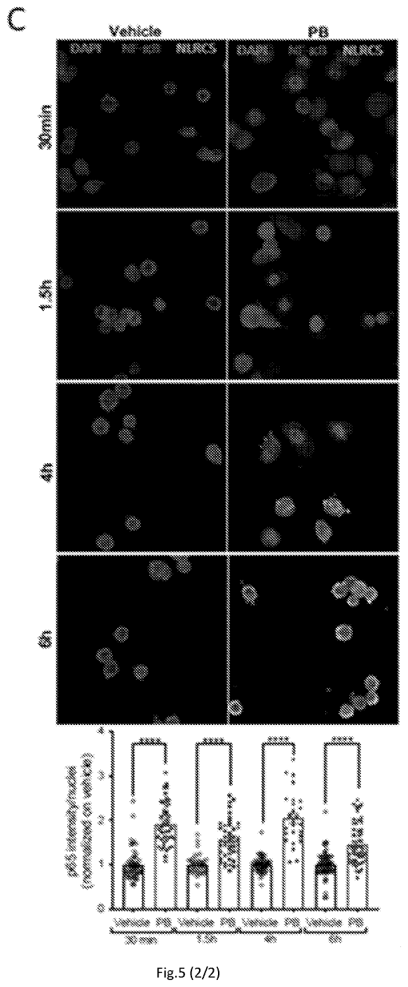

FIG. 5. The RNA expression results highlight the involvement of the innate and inflammatory immunity pathways in postbiotic-mediated HLA class I upregulation

A) Expression of genes correlated to HLA class I antigen processing 24 hours after the treatment with postbiotic (PBLP). The results are expressed as “fold-change” compared to the control carrier. B) Gene ontology of differentially expressed genes after treatment with postbiotic using MSigDB GSEA collections. GOBP: “gene ontology biological processes”, KEGG: “Kyoto encyclopedia of genes and genomes”.

Involvement of NLRC5 in postbiotic-mediated upregulation of HLA class I in the breast tumour cells line SK-BR-3. C) Immunofluorescence analysis of NLRC5 (green) and NF-κB (red) after treatment with postbiotic. Quantification of the intensity of p65 within the nuclei is performed on the entire image and normalized on the control carrier.

The experiments were repeated in triplicate, and a representative experiment is shown. Data represented as mean±SEM. The statistical analysis by 2-way ANOVA *p<0.05, * **p<0.001, ****p<0.0001

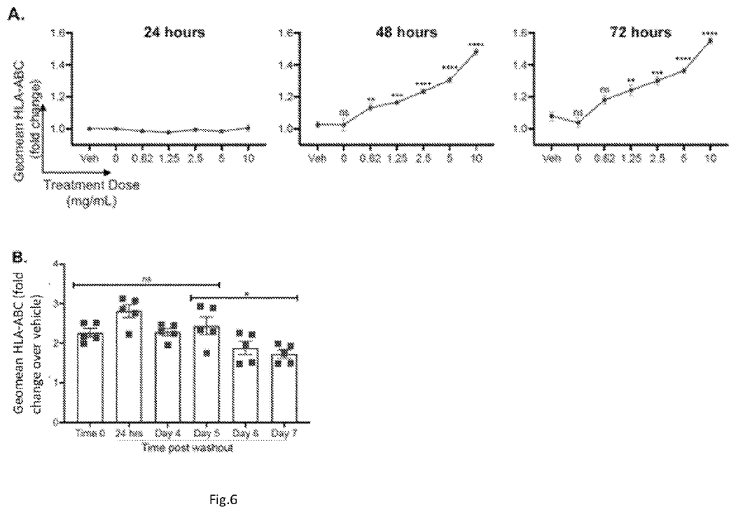

FIG. 6. Postbiotic-mediated upregulation of HLA class I derived from L. paracasei is dose-dependent and transient. A) K562 cells were treated with increasing doses of L. paracasei postbiotic (PBLP) and the samples were taken after 24, 48, and 72 hours incubation to assess the HLA class I expression by flow cytometry. The experiments were repeated in duplicate, and statistical analysis by 2-way ANOVA, **p<0.01, * **p<0.001, ****p<0.0001. B) The Thp1 cells were treated for 48 hours with 1 mg PB or control carrier prior to washing and re-culturing. The samples were taken daily and the geomean intensity of the HLA-ABC expression was measured by flow cytometry (B). Experiment repeated in triplicate. Statistical analysis of 2-way ANOVA, *p<0.05.

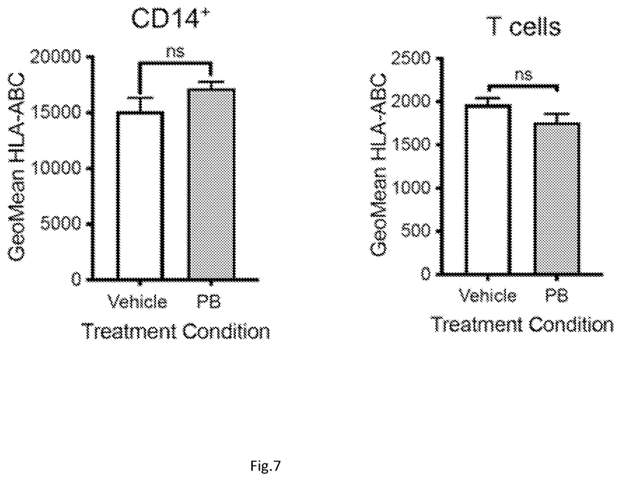

FIG. 7. HLA class I on postbiotic-mediated immune cells (PBLP) L. paracasei. Purified human CD14+ monocytes and purified human T cells were treated with the postbiotic and after 48 hours of incubation the HLA class I expression was assessed by flow cytometry. Experiments repeated in duplicate, statistical analysis by 2-way ANOVA, ns: not significant.

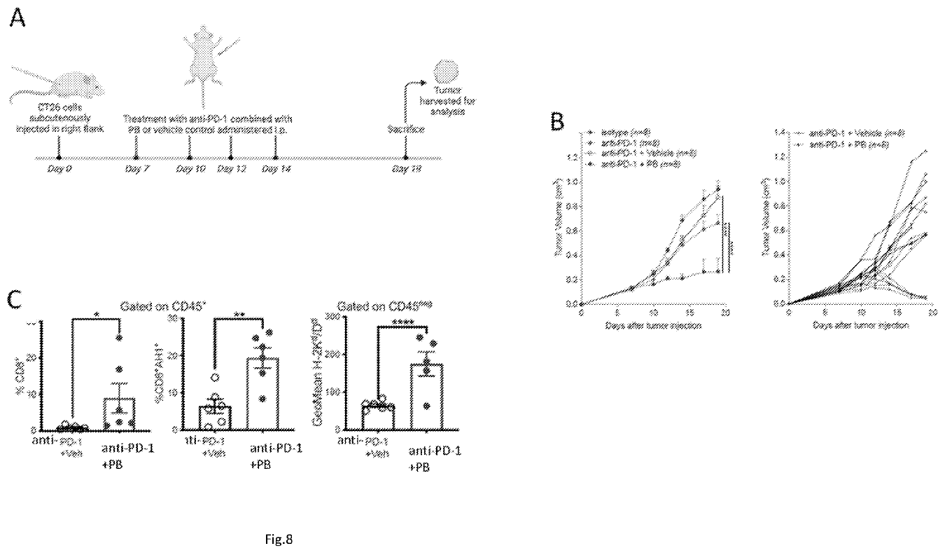

FIG. 8. Postbiotic treatment of L. paracasei(PBLP) combined with anti-PD1 in vivo controls tumour growth and prolongs survival. A) Scheme of the experiment. B) Tumour growth curve for all the treatments (left panel) and individual mice in the carrier and postbiotic (PBLP) treated groups (right panel). C) Flow cytometry analysis of the tumour cells suspension at the end of the experiment (day 19), labelled with anti-CD8, AH1 dextramer, and H-2k^{d}. The geomean and the percentages were measured by flow cytometry. Experiment repeated twice, representative experiment shown. Statistical analysis by 2-way ANOVA (H) or one-way ANOVA (1).

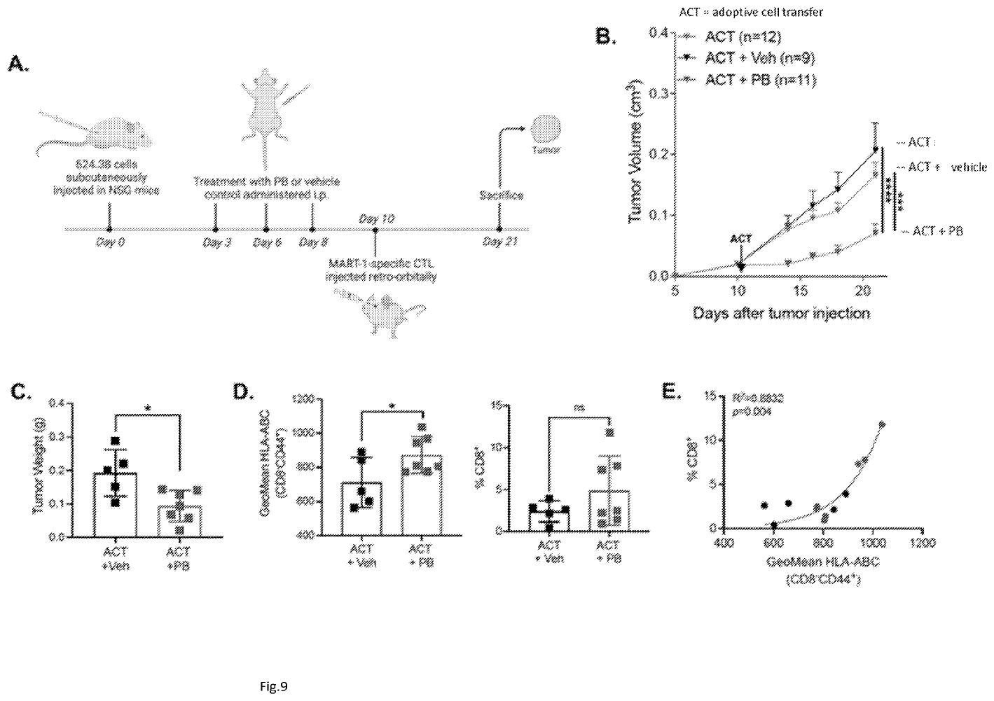

FIG. 9. The postbiotic L. paracasei (PBLP) combined with ACT (adoptive cell transfer) of MART-1-specific T cells controls tumour growth. A) Scheme of the experiment. B) Tumour measurements. C) Tumour weight at the time of the sacrifice. D) The tumours were processed in a single cell suspension and labelled with anti-CD45, anti-CD3, anti-CD8, anti-CD39 and HLA-ABC, geomean and percentages were measured by flow cytometry. Data represented as mean±SEM. Statistical analysis by 2-way ANOVA, *p<0.05, * **p<0.001, ****p<0.0001. E) Correlation of HLA-ABC expression on the tumour cells (CD8⁻CD44⁺) with % CD8⁺ cells in the tumour.

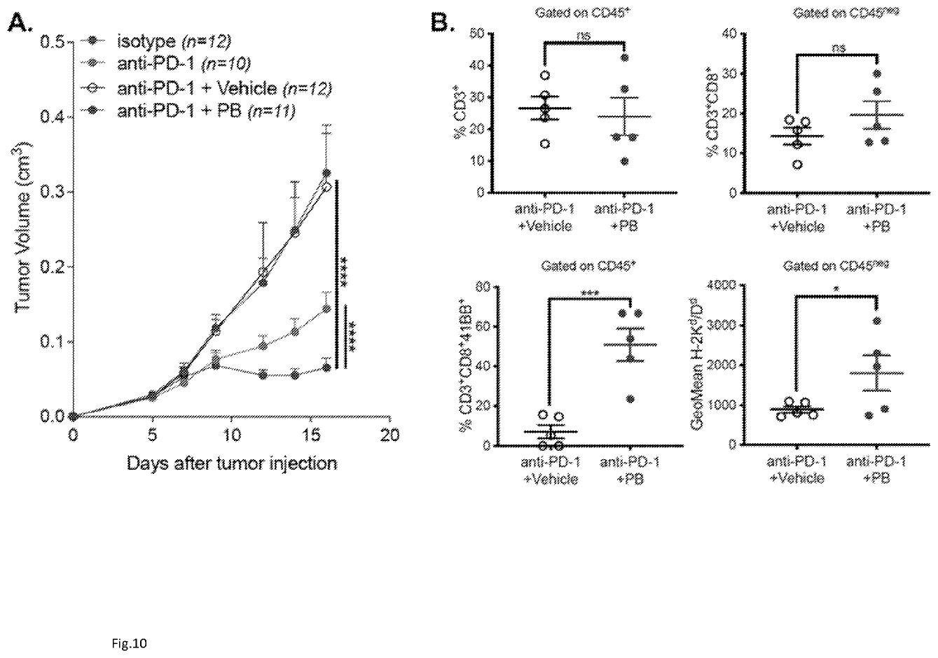

FIG. 10. Postbiotic (PBLP) administered via oral gavage in the 4T1 TNBC model. The mice were randomized into the four treatment groups on day 5 before starting treatment. A) Tumour growth measured by calliper. B) Flow cytometric analysis of the tumour cells suspension at the end of the experiment. Experiment repeated in duplicate, representative experiment shown. Statistical analysis of 2-way ANOVA, *p<0.05, ***p<0.001, ****p<0.0001.

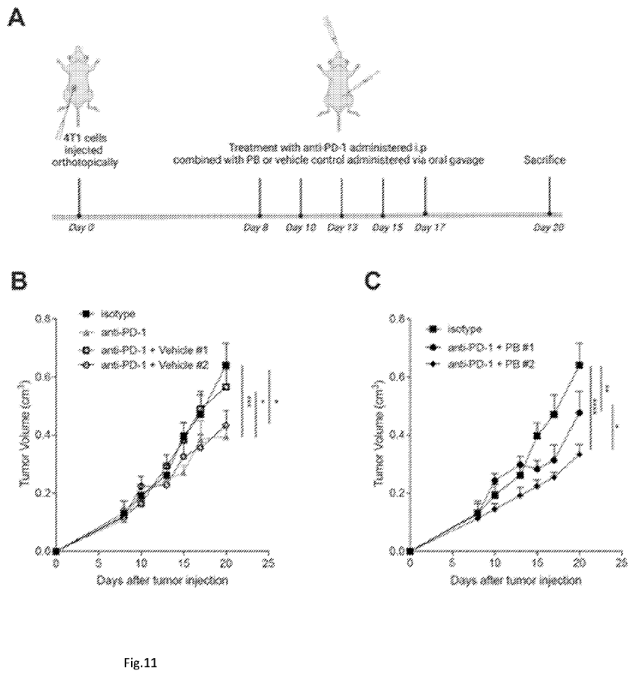

FIG. 11. The treatment with different formulations of L. paracasei postbiotic (PBLP) combined with anti-PD1 in vivo controls tumour growth in the 4T1 TNBC model. A) Scheme of the experiment. B) Graph representing the tumour volume over time for the following experimental groups: isotype, anti-PD-1, anti-PD-1+carrier #1 (Maltodextrin), anti-PD-1+carrier #2 (Mannitol). Statistical analysis by 2-way ANOVA. * p<0.05, **p<0.01, ***p<0.001. C) Graph representing the tumour volume during the experiment for the following experimental groups: isotype, anti-PD-1+PBLP #1, anti-PD-1+PBLP #2. Statistical analysis by 2-way ANOVA. * p<0.05 **p<0.01 ***p>0.001.

### EXAMPLES

### Materials and Methods

### Preparation of the Fermented Product PBBL and PBAM

The supernatant containing the postbiotics of B. longum (PBBL) and A. municiphila (PBAM) were obtained by growing the bacteria, for obtaining the biomass (ON-24 h), in species-specific medium (B. longum—ATCC medium 2107 and A. municiphila—Anaerobe Basal Broth-CM0957-OXOID+mucin type III+glucose-D) and suspending the biomass in a saline solution for 24 hrs at a temperature of about 37° C. The resulting suspension was then centrifuged at 3500 rpm for 10 minutes to eliminate the biomass then filtered with a hydrophilic polyethersulfone (PES) filter membrane with 0.22 μm porosity.

### Preparation of the Fermented Product (Also Referred to as PB or PBLP)

An inoculum of L. paracasei CNCM 1-5220 is grown at a temperature between 32° C. and 37° C. and then placed under soft stirring, so as to avoid oxygenation of the culture medium, in culture broth. Biomass is then allowed to grow for about 16-24 hours. The culture is then centrifuged to separate the bacteria from the culture medium.

The bacteria after centrifugation are transferred into a saline solution with addition of fructooligosaccharides (FOS) for about 24h. At this point the supernatant is separated from the bacteria and heated to 90° C. for 10 minutes (to remove any residue of live bacteria), and added with Glucidex 19D maltodextrin (Roquette) and pulverized by spray drying or freeze-drying.

Mice. Female BALB/c mice aged 6-8 weeks were purchased from Charlies River (Calco, Italy). All the animals were housed in SPF conditions and the experiments were performed according to Italian regulations (Legislative Decree 26/2014) and the EU guidelines (2010/63/EU).

Cell lines. All primary or immortalized human and murine cell lines were cultured in Roswell Park Memorial Institute (RPMI) 1640 medium (Euroclone, ECB9006L) added with 10% FBS (Avantor, 97068-085), 2 mM glutamine (Euroclone BE17-605E), 1% sodium pyruvate (Euroclone, ECM0542D), 1% non-essential amino acids (Euroclone ECB3054D), and 1× penicillin-streptomycin solution (Euroclone ECB3001D) (R-10 medium).

Tumour growth and in vivo treatment. Female BALB/c mice aged 6-8 weeks were inoculated subcutaneously with 5×10⁵ murine tumour line 4T1 (triple negative breast tumour). The tumour measurements were performed every other day using a calliper and the volume was calculated using the following formula: tumour volume=(length×width²)+2. The mice were treated with 250 μg of postbiotic (PB) or control carrier in combination with anti-PD1 (200 μg; clone 29F.1A12, BioXCell) from day 3 from inoculation of the 4T1 cell line and every other day for a total of 4 injections. For survival experiments, the mice were euthanized when the tumour volume had reached 1 cm³.

Tumour infiltrate analysis. The tumours were collected 18 days after inoculation and processed to obtain a single-cell suspension by means of a 70 μM filter. The cells were washed 2 times with PBS and labelled with anti-CD45, anti-CD3, anti-CD8, anti-CD4, anti-CD39 and anti-H-2k^{d} (Biolegend) and the Fixable Live/Dead Viability Dye BV510 (1:1000, BD Bioscience) for 15 minutes at 2-8° C. The cells were washed twice and resuspended in bufferfor flow cytometry (0.1% BSA and 2 mM EDTA in 1×PBS) before performing FACSCanto II analysis.

Splenocyte activation test. The spleens were taken 18 days after tumour inoculation and processed to obtain a single-cell suspension by means of a 70 μM filter. The red blood cells were lysed with a lysis buffer for RBC (Ammonium Chloride) for 10 minutes, and washed 2 times with PBS. 2×10⁵ splenocytes were plated at a 1:1 ratio with the tumour line 4T1 in 96-well U-bottom plates (Falcon) for 24 hours. After co-culture, the cells were washed with PBS and labelled with antibodies specific for CD45, CD3, CD8, 41BB (Biolegend), and the Fixable Live/Dead Viability Dye BV510 (BD Bioscience). The cytotoxic T cell activation (CD8⁺41 BB⁺) was measured by FACSCanto II.

Treatment with postbiotic in vitro. 2-3×10⁵ tumour cells were plated on 96-well plates and treated for 48 hours with 5-10 mg/mL of postbiotic or control carrier in R-10 culture medium. The samples were washed once with PBS and labelled with anti-HLA-ABC (1:200, clone W6/32, Biolegend) or H-2k^{b}(B16) and H-2k^{d} (4T1) (Biolegend) and the Fixable Live/Dead Viability Dye BV510 (1:1000, BD Bioscience) for 15 minutes at 2-8° C. The cells were washed twice and resuspended in buffer for flow cytometry (0.1% BSA and 2 mM EDTA in 1×PBS) before performing FACSCanto II analysis.

Degranulation test. Cytotoxic T lymphocytes (CTLs) specific for the MART-1²⁶⁻³⁵ antigen were incubated with the Thp1 cell line, in the presence or absence of the MART-1²⁶⁻³⁵ peptide or with tumour cells, both treated with carrier or Postbiotic for 5 hours at 37° C. in the presence of anti-CD107a (1:200, BioLegend 328618), brefeldin A 10 μg/mL (1:1000, Biolegend 420601), and GolgiStop with monensin (1:1000, BD Biosciences 554724) in 200 μL of R-10 culture medium. After incubation, the cells were washed once with PBS and labelled with antibodies directed against CD8 (Biolegend 344708) and the Fixable Live/Dead Viability Dye BV510 (1:1000, BD Bioscience 564406). The cells were washed twice and resuspended in buffer for flow cytometry before performing FACSCanto II analysis.

Immunofluorescence. The SK-BR-3 cells treated, as described, with carrier or postbiotic were plated at a concentration of 200,000 cells/mL on poly-D-lysine treated polarized microscope slides in sterile plates to avoid contamination. After 4 hours, the samples were washed with PBS and fixed with 4% PFA for 20 min. The cells were rehydrated and blocked with 0.1 M Tris-HCl pH 7.4, 2% FBS, and 0.3% Triton X-100. Staining was performed with anti-NLRC5 antibodies (1:100, Santa Cruz Biotechnology sc-515668) and anti-lipopolysaccharide goats (1:100, Antibodies-online, ABIN479062) over-night. The samples were washed with 0.1 M Tris-HCl pH 7.4, 0.5% Tween20, and labelled with the conjugated antibody AF647 donkey anti-mouse (Thermofisher, Monza, Italy, A21202). The nuclei were labelled with DAPI (1:45,000, Thermofisher, 1306). The sections were mounted with Vectashield mounting medium liquid and analysed with Leica TCS SP8 Laser scanning confocal microscope HC PL APO CS2 40X/1.30 with oil immersion lens (Leica). All the images were analysed with Fiji (ImageJ) version 2.3.0 software.

Phosphorylation analysis of STAT1 and NF-kB. The SK-BR-3 cells were plated on 96-well plates and treated for 30 minutes, 2 hrs, 4 hrs, 6 hrs and ON with 10 mg/mL of postbiotic or control carrier in culture medium R-10. The samples were fixed with CytoFix following the BD Phosflow™ protocol (Becton Dickinson). The samples were then labelled with anti-pSTAT1 and anti NF-kBp65 (Biolegend) for 30 minutes at room temperature. The cells were washed twice and resuspended in buffer for flow cytometry (0.1% BSA and 2 mM EDTA in 1×PBS) before performing FACSCanto II analysis.

### Generation of MART-1-Specific T Cells.

The CD8+ (cytotoxic T lymphocytes, CTL) and CD14+ (monocytes) cells were isolated from peripheral blood mononuclear cells (PBMC) from healthy HLA-A*02 donors using magnetic beads following the manufacturer's instructions (Miltenyi Biotec). The CD14+ cells were differentiated into monocyte-derived dendritic cells (moDC) using 800 U/mL IL-4 (Peprotech) and 1000 U/mL granulocyte macrophage colony stimulating factor (GM-CSF; Peprotech) for 5-6 days. moDCs were pulsed with 10 μM MART-1 peptide for 2 hours, washed, and plated with autologous CD8+ T cells (10:1 moDC:CD8 ratio) in a 24-well plate. After 10-12 days, the CTLs were restimulated with autologous monocytes pulsed with MART-1 peptide. The CTLs were fed every 2-3 days with 50 U/mL IL-2 or 10 ng/mL IL-15, if necessary.

Production of the Postbiotic Formulations Derived from Lactobacillus paracasei (PBLP)

After separation from the biomass according to the process described above, the recovered supernatant is heated to 90° C. for 10 minutes (to remove any residue of live bacteria), and was added with 5% wN Glucidex 19D (Roquette) maltodextrin or pharmaceutic form of D-Mannitol—PEARLITOL 160 C mannitol (Roquette). Then, the formulations were pulverized, by spray drying or freeze-drying.

### Results

The in vitro postbiotic treatment upregulates the HLA class I expression on the melanoma and breast tumour cells lines

Recent literature has reported a potential role for the gut microbiota and the response to the treatment with anti-PD1 mAb immunotherapy in patients with solid tumours (Gopalakrishnan 2018, Routy 2018, Matson 2018). Furthermore, the down-regulation of HLA class I expression by tumour cells is an immune escape mechanism that has been well described in melanoma (Garrido 2016, Sade-Feldman 2016, Zaretsky 2016) and, to a lesser extent, in breast cancer (Kaneko 2011). Because of the paucity of literature on HLA class I expression of breast cancer, we first sought to confirm whether HLA class I expression of breast cancer played a role in patients' overall survival by querying publicly available TCGA datasets (FIG. 1A).

To investigate whether the postbiotics secreted by bacteria could influence the HLA expression on the tumour cells, we generated postbiotics from two bacterial strains identified by Matson et al. and Routy et al. (B. longum and A. municiphila, respectively) as enriched in anti-PD-1 responsive patients, as well as a strain of L. paracasei previously identified by the inventors as protective and immunogenic (Mileti 2009) (FIG. 1B).

The inventors first tested said probiotics on a breast tumour cells line expressing low levels of HLA class I, SK-BR-3, and found that the postbiotics released from L. paracasei (PBLP) can induce a significant and dose-dependent increase in HLA class I surface expression, while the postbiotics derived from A. municiphila (PBAM) and B. longum (PBBL) have minimal efficacy. The inventors chose to investigate beyond the efficacy of PBLP on further breast tumour cells lines (FIG. 1C), and on immortalized (FIG. 1D) and primary (FIG. 1E) melanoma cells. In addition, the PBLP treatment of murine melanoma (B16F10) and breast cancer (4T1) cell lines upregulated the MHC H-2 surface expression (FIG. 1F). The postbiotic treatment upregulated the HLA class I expression in the tested cell lines.

### The Postbiotic-Treated Tumour Cells Induce TA-Specific CTL Responses

To verify whether the postbiotic PBLP induced HLA class I expression results in an increased activation of TA-specific T cells (tumour antigens), we selected two primary melanoma cell lines, one of which expresses the MART-1 antigen, and we pre-treated them with postbiotics for 48 hours. After pre-treatment, the melanoma cells were stained for HLA class I surface expression (FIG. 2A) and tested for their ability to induce effector responses from a MART-1²⁶⁻³⁵-specific polyclonal CTL.

As expected, the primary melanoma cells that do not express the MART-1 antigen (HsMel12) did not stimulate a CTL response regardless of the postbiotic treatment and the primary melanoma cell line HLA-A2+ expressing MART-1 (HsMel13) was able to induce a moderate response by the MART-1²⁶⁻³⁵-specific CTL. Interestingly, the pre-treatment of HsMel13 with the postbiotic PBLP resulted in an increased activation of the MART-1²⁶⁻³⁵-specific CTL, as measured by the CD8⁺ CD107a⁺ percentage (FIG. 2B). The addition of an HLA class I blocking antibody (anti-W6/32) resulted in the abrogation of MART-1²⁶⁻³⁵-specific CTL activation (FIG. 2B), indicating that the postbiotic treatment elicits an HLA-dependent T cell response. In addition, the treatment with the postbiotic PBLP of the melanoma cell line 624.38 leads to an increased cytotoxicity by the MART-1²⁶⁻³⁵-specific CTLs (FIG. 2C).

To directly validate the upregulated surface HLA class I expression, the immunogenic peptide MART-1²⁶⁻³⁵ was added exogenously to the Thp1 cells (of monocytic origin) that were pre-treated with the carrier or with the postbiotic and tested for their ability to increase recognition by the MART-1²⁶⁻³⁵-specific polyclonal CTLs. The exogenously added MART-1²⁶⁻³⁵ peptide is loaded onto the Thp1 class I HLA molecules, therefore, postbiotic-induced upregulation of HLA class I surface expression will bind multiple activated MART-1²⁶⁻³⁵ peptides, leading to an increase in the number of MART-1²⁶⁻³⁵ specific CTLs activated.

As expected, the treatment with the postbiotic upregulated the surface expression of HLA-A2 on Thp1 (FIG. 2D), which subsequently potentiated the antigen-specific CTL recognition (FIG. 2E, +MART-1²⁶⁻³⁵ peptide). It is important to underline that the postbiotic-treated Thp1 did not induce a non-specific basal activation of CTL (FIG. 2E, no peptide). Collectively, these data show that upregulation of HLA class I surface expression of the tumour cells is a mechanism by means of which the postbiotic PBLP can increase the recognition of the tumour cells by TA-specific CTLs.

The Postbiotics Combined with Anti-PD-1 Cancel Tumour Growth In Vivo in a Mouse Model of TNBC

Published case studies have highlighted a link between immune evasion of the tumour cells after ICI treatment via down-regulation and/or loss (Sade-Feldman 2016) of HLA class I. To assess whether the postbiotic can increase the TA-specific immune responses when combined with ICI, the female BALB/c mice were orthotopically injected with 4T1 murine TNBC cells and treated with anti-PD-1 mAb in combination with the postbiotic or the carrier (FIG. 3A). The postbiotic treatment significantly reduced the growth of the primary 4T1 tumour compared to the carrier-treated group (FIG. 3B). The tumours taken from mice treated with the postbiotic PBLP had an increased percentage of CD8⁺ T cells (FIG. 3C) and upregulated the surface MHC class I antigen expression compared to the tumours isolated from the mice treated with the carrier (FIG. 3D), confirming the results obtained in vitro.

The percentage of CD8⁺CD39⁺ cells, suggested to have tumour specificity (Duhen et al; Nature Communication 2018), were also correlated to MHC class I antigen expression (FIG. 3E), suggesting that the postbiotic-induced upregulation of MHC class I antigens results in an increased T cell activation toward the tumour. To assess whether the treatment with the postbiotic induced TA-specific T cell responses, the splenocytes were isolated from postbiotic and carrier-treated mice and restimulated in vitro with 4T1 tumour cells. As shown in FIG. 3F, the mice treated with the postbiotic had a higher percentage of TA-specific CD8⁺ splenocytes, an effect that may be due to in vivo postbiotic-induced upregulation of MHC class I antigen.

In addition, the combination of the treatment with the postbiotic and the anti-PD-1 antibody prolonged mouse survival (FIG. 3G), compared to the control carrier cohort. Taken together, these results suggest that the postbiotic PBLP may upregulate the expression of the MHC class I antigen of the tumour surface, increase the number of TA-specific CD8+ T cells, and prolong survival. These results highlighting its potential use as a combination therapy with ICI.

### Mechanism for the Postbiotic-Induced Upregulation of HLA Class I

The inventors, to understand the mechanism by which the postbiotic PBLP induces HLA class I upregulation, assessed the role of NLRC5, which previous studies have shown to be critical for basal and IFNγ-induced HLA class I expression (Neerincx 2012). To verify whether NLRC5 was induced by the postbiotic treatment, the inventors incubated the SK-BR-3 cell line with postbiotics for 48 hours before measuring NLRC5 expression by immunofluorescence (FIG. 4A).

After observing a marked upregulation, the inventors assessed whether the presence of leptomycin B, which prevents the transfer of NLCR5 between the nucleus and the cytoplasm via CRM1 (Neerincx 2012), could influence the postbiotic-mediated upregulation of HLA class I. The addition of leptomycin B completely cancelled the effect of the postbiotic (FIG. 4B), indicating that NLRC5 plays a fundamental role in the postbiotic-induced upregulation of HLA class I expression.

Since the JAK-STAT pathway is strongly involved in the HLA class I modulation (Rodriguez 2007) and has been implicated in the regulation of NLRC5 (Jongsma 2019), the inventors sought to understand whether the HLA class I postbiotic-mediated upregulation was mediated by this pathway. The analysis of the gene ontology of the SK-BR-3 cell line treated with the postbiotic suggested the association of the JAK-STAT pathway at the RNA level (FIG. 4C). However, when this cell line was treated with the postbiotic in vitro, no STAT1 phosphorylation was observed (FIG. 4D), requiring JAK-STAT-dependent HLA class I expression (Christova 2007). Because of these results, the inventors examined the RNA sequencing data using Ingenuity Pathway Analysis (IPA). This analysis highlighted the involvement of the inflammatory pathways linked to the innate immune system, including the inflammatory pathway NF-KB. The in vitro treatment with the postbiotic induces NF-KB phosphorylation (FIG. 4E). To understand whether the NF-KB activation is involved in the upregulation of HLA class I, we inhibited its activation by using a specific inhibitor. As can be seen in FIG. 4F, blocking NF-KB activation abrogates postbiotic PBLP-mediated upregulation of HLA class I expression, validating the role of NF-KB.

The Treatment with the Postbiotic Upregulates the HLA Class I Locus and the Innate Inflammatory Pathways

To further investigate the mechanism by which the postbiotic can upregulate the HLA class I expression, the inventors first assessed gene modulation by RNA sequencing of the breast tumour cells line SK-BR-3 after treatment with L. paracasei postbiotic (PBLP) or the control carrier. As shown in FIG. 5 panel A, the sequencing results confirmed the upregulation of the entire locus of the class I HLA molecules (A, B, and C) and the antigen processing mechanism (APM) (proteasome subunits and transporters associated with the antigen processing molecules [TAP]) at the RNA level. In addition, the inventors took a closer look at the differentially expressed genes using the Gene Set Enrichment Analysis (GSEA) software, which highlights the upregulation of several inflammatory pathways including highlighting the involvement of inflammatory pathways, including the signalling cascade of the toll-like receptor (TLR) and NF-κB (FIG. 5B). Since NF-κB was the most modulated pathway, the inventors investigated the involvement of NF-κB and observed that the p65 isoform is phosphorylated (FIG. 4E) and translocates into the nucleus (FIG. 5c) after the treatment with the postbiotic of L. paracasei (PBLP) in vitro.

The Postbiotic-Dependent Upregulation of HLA Class I is Dose-Dependent, Transient; the Postbiotic does not Influence the HLA Class I Expression in the Immune Cells

The inventors tested the dose-dependent effect of the postbiotic of L. paracasei (PBLP) on the myeloid cell line, K562 expressing low basal levels of HLA class I, and found that the postbiotics released from L. paracasei (PBLP) can induce a significant, dose-dependent increase in the surface HLA class I expression (FIG. 6A).

In addition, to verify the duration of the postbiotic-induced upregulation of the HLA expression, the inventors incubated the monocytic tumour cell line Thp1 with the postbiotic for 48 hours before washing and continuing the culture in a medium without postbiotic. FIG. 6B shows that Thp1 HLA class I expression remains elevated for five days after removal of the postbiotic, before starting to decline between days 5 and 7. These results highlight the transient nature of the HLA class I upregulation. To understand whether the postbiotic of L. paracasei (PBLP) could upregulate the HLA class I expression even on immune cells, the CD8+ T cells and the antigen-presenting cells (CD14+ monocytes) were treated with postbiotic of L. paracasei (PBLP). As shown in FIG. 7, the postbiotic of L. paracasei (PBLP) has little or no effect on the HLA class I expression on T cells and on the monocytes.

The Treatment with Postbiotic of L. paracasei (PBLP) Combined with Anti-PD1 In Vivo Controls Tumour Growth and Prolongs Survival in a Mouse Model of CRC

The inventors then assessed whether the synergistic effect of the postbiotic of L. paracasei (PBLP) and the treatment with anti-PD-1 was found in other tumour model systems, i.e. the CT26 mouse model of CRC (FIG. 8A). Similar to model 4T1 (FIG. 3), the treatment with postbiotic of L. paracasei (PBLP) combined with anti-PD-1 significantly attenuated tumour growth (FIG. 8B) with respect to the control carrier. In addition, the inventors observed that the combined treatment was very efficient in the CT26 model, as the tumour regressed in 5 out of 8 mice, 2 of which showed complete tumour regression. The flow cytometric analysis of the tumours processed at the end of the experiment showed a slight increase in the CD8⁺ T cells in the tumour microenvironment, with a significant increase in AH-1⁺ dextramer positive CD8⁺ T cells (FIG. 8C). Since AH1 is a known antigen of the CT26 model this indicates that the treatment with postbiotic of L. paracasei (PBLP) is enriching for tumour-specific T cells at the tumour site.

The Postbiotic of L. paracasei(PBLP) Combined with ACT (Adoptive Cell Transfer) of the Specific MART-1 T Cells Controls the Xenogeneic Tumour Growth in NOD Scid Gamma (NSG) Mice

In addition to ICI treatment, the immunotherapy with adoptive cell transfer (ACT) of TA (Tumour Antigen)-specific T cells is increasingly being used as a treatment option for melanoma patients. The HLA class I expression on the tumour cells is fundamental to the effectiveness of ACT; therefore, the inventors sought to understand whether the postbiotics could control tumour growth in this system, as well. To do so, NOD scid gamma (NSG) mice were injected subcutaneously with the human melanoma cell line 624.38 Mel prior to treatment with the postbiotic of L. paracasei (PBLP) or the control carrier (FIG. 9A). Once the tumours were palpable, the mice were infused with MART-1-specific polyclonal CTL (see Materials and Methods). A greater control over tumour growth was seen when the mice were pre-treated with the postbiotic compared to the group that received the control carrier (FIG. 9B), and the tumours weighed significantly less at the end of the experiment than the control (FIG. 9C). Importantly, the tumour growth was similar among all the treatment conditions prior to ACT, indicating that the postbiotics do not directly act on tumour growth, blocking or slowing growth (FIG. 9B, from day 5 to day 10). Similar to the results seen in vitro and in 4T1-TNBC models, CT26-CRC in vivo (FIG. 1, FIG. 3, FIG. 8), the treatment with postbiotic upregulated the HLA class I expression on the tumour cells when compared to the control carrier group (FIG. 9D). The increase in the CD8⁺ T cells in the tumour microenvironment, although not statistically significant (FIG. 9D) showed a significant correlation between the percentage of infiltrating CD8⁺ T cells and the HLA class I expression of the tumour cells (FIG. 9E).

### The Postbiotic Administered Via Oral Gavage in the 4T1 TNCB Model

The inventors also treated mice bearing 4T1 with the postbiotics administered orally via oral gavage instead of i.p. (intraperitoneal) injection, for a more translational approach. The treatment with the postbiotic of L. paracasei (PBLP) via oral gavage led to a tumour growth control (FIG. 10A), recapitulating the results obtained by i.p. injection (FIG. 3, FIG. 8, FIG. 9). While there was a trend of CD8⁺ T cell infiltrate increase in the tumour, the CD8⁺ T cells showed a more activated phenotype (measured as 41 BB expression) and an upregulated MHC class I expression was observed in the tumour cells in the mice treated with postbiotic (FIG. 10B).

Effects of Different Formulations of Postbiotic of L. paracasei (PBLP) Combined with Anti-PD1 In Vivo in Controlling Tumour Growth in TNBC 4T1 Model

The inventors assessed whether the postbiotic formulation of L. paracasei (PBLP) containing maltodextrins (PBLP #1) compared to one containing mannitol (PBLP #2) could increase TA-specific immune responses when combined with ICI; female BALB/c mice were orthotopically grafted with 4T1 murine TNBC cells and treated with anti-PD-1 monoclonal antibody (mAb) combined with one of the postbiotics (PBLP #1 maltodextrin formulation, PBLP #2 mannitol formulation) or the respective control carrier (carrier #1—maltodextrin and carrier #2—mannitol, respectively) (FIG. 11A). As shown in FIG. 11B, the inventors showed that the treatments with the two different carriers (in combination with anti-PD-1) had significantly different effects on the primary 4T tumour growth compared to the isotype-treated control group or to the anti-PD-1 control group. The effect of the ICI treatment is fundamentally lost after oral administration of the Carrier #1 suggesting that the Carrier #1 had a negative effect on tumour growth. In contrast, the inventors showed that the carrier #2 had no adverse effects on the ICI-treated group. These data indicate that the carrier #2 does not alter the therapeutic effect of ICI treatment alone. Finally, in FIG. 11C, the inventors showed that the postbiotic PBLP #2 has a better effect on tumour growth inhibition than PBLP #1. Taken together, these results suggest that both postbiotic formulations administered by oral gavage can control tumour growth, highlighting their potential use as combination therapy with ICI. However, the PBLP #2 formulation showed better results in controlling tumour growth and is preferable over the PBLP #1 formulation.

### Sequences

Also included in the present invention are nucleic acid sequences derived from the nucleotide sequences shown below, e.g., functional fragments, mutants, derivatives, analogues, and sequences with a % of identity of at least 70% with the sequences below.

Five gene sequences are described below, representing specific “core” genes of the species L. paracasei and L. casei.

SEQ ID 1
ATGAATCAAAAAGCTTTGAATCAATTTCCTGAACTTACCTACACAGAACAAGTGTCGGTTGTTGGC
GGCGATTTGTCAGTCGAAGTCATCATGAAAGGTATCTTCACCGGTATCTTTGATGCTGGGTACCA
AGTGGGTCAGTCAATCGCAAAATGGGTTAAGTAA
SEQ ID 2
ATGAAAGGTAAGCGGCAGCACTTACTTTTATATTTTGTTCTGGGTATGATGACAGGACTGGTGAC
GGCAGCGATTTTTCATATCATTTATGCCTGGCTTTTTCATTGA
SEQ ID 3
ATGCCCAAAAGGGTCGATCAACATATACGTTCACGCCTTAAAGGCTTTACTTTAATTGAAGTGGT
GGTCAGCCTGATTTTACTTGCGGCGGTCATGCTGTTATGGCGACCGGTTTTATTGCATGTCACGC
GGTTCACGCTTCAAGACCATGTGCTAATCACGTCATTGCAAGCAGAGCATGACTTGCAAATGTTT
GTACGAGATAAAAAGTTGCGGTCTGTGGCCTTAATGTCGGTAAGGGTGAGAAGTCCCGAGAAAG
CTTACACGATCAATTTTTATCAGACCAAACATTTTCGCGGTATGGTTCGTGTGATGGGATCTGAAA
ATGGGCATATGCCATTATTTACGCATCTAACCGGTGTCAATTTTAGCAAGGTAGCTCAAGGCTTT
CGCTATCGTCTGTATTTGACGACTTCGCAGAAGATTGACGGTGGGGTGCAAATCGATGAAGATA
CGCGGTAG
SEQ ID 4
ATGGCCGCTGATTTCACCCAATTACAACAAGCCATTCGCTTGCTCAATGCCCATACTCGAGCTGC
TGATGAGCAAGCGTGGCAAGTGCTTTTTGATCGTTGGCTGGCAACTTTATCCTCTGAAACTCGCC
GGCAAATGCAAACAGTTCGGTTTAATCATGCCCAATTGACGTTACTCACAACGCTGGATCAAAGC
AGTCGCAAACAACTGCGCAATCAGGATTTAACCGCTGCTGTTCCGTTCTCACAAGGCCTAGTCTC
ACGCTATGTTGCTCGCCTTGTTCAATTAAACTTGCTGACAAAATTATCCTTGCCCGACAATCGCAA
GGCCTACATTGTTGCACTAACTCCGCTTGGTCAACAAGTCGCTGCCTTACATCAGCAAATGCATC
ATCACACAAATGCTCAACTCGCCTCTGTACTTCATACCCTTGATCCACAAGATGTTCAAACTACCA
TTCAGGTACTCACAAAACTGACGGCTCAGCCTTTACATCCCAAGTCTTAG
SEQ ID 5
ATGGGCGGTGTCATTTGTTACGCGGTGCCGGTCTTTTGGAAAAGAATACTTCGCAGACACCTGAT
TCACGAGATTAAGACCCTGAATCAAGGATTGCAGTTATCAAGCAAAGCCATGAGCCAATTAATTG
ATCCGGAAAATCCTTATATGGTATTTGCTGATGAAAATGGTGAACTGGATTTTTCATTTTTGTGGC
TAGGCAACTTGCGTCAATTGAGGCGTGAACTGCGTCTAATTAAAGAACAGAAAGCTAGGGTTTGA

The following are the unique sequences of L. paracasei B21060 relative to the publicly available L. paracasei species in the NCBI databases. SEQ IDs from 6 to 8 refer to the gene sequences, and SEQ IDs from 9 to 18 refer to the DNA sequences of the genome.

SEQ ID 6:
ATGAGTAAATATAAAGTTATTATTTGGGGATTAGGAAACGTTGGTCGTTCCGCAGTGAGAATGAT
CGCGGAAAGACAAAATATTTTTGAATTGGTTGCAGCCGTTGACGTTGATCCAAAGAAGTTAGGTA
AGGATGCCGGAGAAGTCTTTGATTTTGACAAAGTCGGCGTCAAAGTTTCAGATGATATTGATGCA
GCCTTGAAACTTCCAGCTGACATTGTGCTCGACTTTTGCCCAACGGAAATGGACAAACAAGGAAC
ATTCATGCCTTCTGCTATTCGACTCGCCAAATCGCTCGATGCCGGTAAAAACGTTATTACCACGA
TTCCGGTATATCATGTTCAAGACAGTCAGCCAGAAGTATATGAATATCTAAATGAACATGCTAAAG
CACATAATGTTGCTTTTGTACCATTTGGACTTTTGCCAGGCGATTATGCCTCATATATCCCACTAG
TTTTGGCCGGGGCCATGGGCCACGTGGATAAAATTGTTGTTCAATCCGGTGAAGATGACTGGCA
CAACACATCAGGCTGGGTCGATGTCTTCTCATATGGCGGCGATATCAATAAATATCCAAAACCAG
ACTCAGACGAAGATCTCTTGGCTAAGTTCATTTATGCTTATTATTCATCCGGCGTATACGAGATGG
CCGATAGGATCGGTCTGAAATATGATACCTTCAAACCAGAGCATGAAGTCTTCACTGCACCCAAA
GATTTGGAAACGATCAAGGGTACAGTCAAAAAGGGCAGCATTTATGCCCACAGATTTACCATGGC
ACTTTACAACGGCAACGAACAAGTAGCCGCCTTAAGATATGTTCATAAAGTTGATAATAAAGAGA
CACCAGAATTACCGATCAATAATACGATTCATATTGAAGGCTTGCCGTCAGTCGATGCGCAGATC
GATGGATTGATCCCAGAAAGAGAAGGCTACGTTTCATCAGCCGCTCCAGCAGTCAACTTGATCC
CTAGCATTCTCGAGACCGACAAGACAGGTTATGTTGAAGTCTGCGACCTTCCAGTAGTGATTGCC
AGGCCATTGGATATTGGCGCAAAAAAATTAGTCTAG
SEQ ID 7:
ATGGCAACCTATTCGCAGATAGAACTAGACATAATCAAATCATTTAAAGGGCTGATGAAAGACCA
TGAATTCACTGAGATCTCAATTAAAATGATCGCTGAAAAAGCCGATATCACTCGACGCGGCTTTTA
CAATCACTTCTTAGATAAATATGATCTTGTCAGTACCATCTTTGAGCATGATCTTTTTCCAACAGTC
ATCAGTTTGACGAATATCAATGACTGGGATCAAGGGTCGCTGTTTATCGTGAATTATCTCCAAGA
CAATCGCGACTACTATAAAAAATTGTTGTCGCTTGAAGGACAAAACTGTTTACAGACAGACTTTTA
TAAATTGACTGAGATGCAGATTGGGATCTTGATCCCAGAAATATTGGTCGGTAGGAAAATTTCTG
ACGAAGATCAGGCATTTTTAAGCGATTATTATTTTCACGCTTATATGGGACTGACTACCGAATGGG
TCAAAGGTAAATATGGTTTTTCAACTCAGGAGTTCGTTAAACGGTGGAAAGCCTTACTCAATAATT
CAATGCATAATTATCTGGACAACTACGCTCGATGA
SEQ ID 8:
ATGAGTAAATATAAAGTTATTATTTGGGGATTAGGAAACGTTGGTCGTTCCGCAGTGAGAATGAT
CGCGGAAAGACAAAATATTTTTGAATTGGTTGCAGCCGTTGACGTTGATCCAAAGAAGTTAGGTA
AGGATGCCGGAGAAGTCTTTGATTTTGACAAAGTCGGCGTCAAAGTTTCAGATGATATTGATGCA
GCCTTGAAACTTCCAGCTGACATTGTGCTCGACTTTTGCCCAACGGAAATGGACAAACAAGGAAC
ATTCATGCCTTCTGCTATTCGACTCGCCAAATCGCTCGATGCCGGTAAAAACGTTATTACCACGA
TTCCGGTATATCATGTTCAAGACAGTCAGCCAGAAGTATATGAATATCTAAATGAACATGCTAAAG
CACATAATGTTGCTTTTGTACCATTTGGACTTTTGCCAGGCGATTATGCCTCATATATCCCACTAG
TTTTGGCCGGGGCCATGGGCCACGTGGATAAAATTGTTGTTCAATCCGGTGAAGATGACTGGCA
CAACACATCAGGCTGGGTCGATGTCTTCTCATATGGCGGCGATATCAATAAATATCCAAAACCAG
ACTCAGACGAAGATCTCTTGGCTAAGTTCATTTATGCTTATTATTCATCCGGCGTATACGAGATGG
CCGATAGGATCGGTCTGAAATATGATACCTTCAAACCAGAGCATGAAGTCTTCACTGCACCCAAA
GATTTGGAAACGATCAAGGGTACAGTCAAAAAGGGCAGCATTTATGCCCACAGATTTACCATGGC
ACTTTACAACGGCAACGAACAAGTAGCCGCCTTAAGATATGTTCATAAAGTTGATAATAAAGAGA
CACCAGAATTACCGATCAATAATACGATTCATATTGAAGGCTTGCCGTCAGTCGATGCGCAGATC
GATGGATTGATCCCAGAAAGAGAAGGCTACGTTTCATCAGCCGCTCCAGCAGTCAACTTGATCC
CTAGCATTCTCGAGACCGACAAGACAGGTTATGTTGAAGTCTGCGACCTTCCAGTAGTGATTGCC
AGGCCATTGGATATTGGCGCAAAAAAATTAGTCTAG
SEQ ID 9
Position 102558 . . . 102986
AAAAACGGCTTAGAACGCTCATATTTGCGTTCTAAGCCGTTTTTATCAGCATAGGTTCTTGACACC
AATAAACATCTTTAGTAATTGATCAAATTTAGGCAATGTGCTTTTGTCGGTGAATGGCGATAGCCC
TACCGAAGCTTCAGCTGAGGTTCTTCTGAGCCACGCAAGCGAAGCGCGCTAGGGCAAGCCAAC
GGCGCGCAGGCGAAGCCGGAGTTAAATGTGGCGCAGCCACACCTTTTTAGGGAGCAACGCGAC
CAGAATTTTGTATGGGGTTTGGGAAGAGGTTCTCCCCAAGGTCTTTTGTGGTTATTAACAAGCAA
AACACAAACACAAGCCTCGCGCGCGTTATATATACTTCTAAATACTTTTAAATACTTTAAGTACTTA
GGGAGACGAGAATGGCTCAACCACGCGTTTAAATCGACT
SEQ ID 10
Position 103624 . . . 103864
ACGACCTCTCGACCACCCACTGCCTCACCAATCCCCAGGTGAACCGGGCCAAGGGCACTACCG
AGCAACCCGACCCCTATATCCCGGTGGGCGTGGTGAAGCAGACCGATGGGGGCATCGTGGTGC
GGGGCGCGCGGATGCTCTCCACGCTGCCCACGGCGGATGAGCTTTTAGTCTTCCCCAGCACTT
TGCTCAAAGAAGGGCCGGGAGCCGACAAGTACGCCGTGGCCTTCGCCATCCC
SEQ ID 11
Position 254291 . . . 261674
ATTGATCGCCTCCGGGTCACTTATATGTAACTAATAATACTCCCTTCTCTCTTTAGTTACAATAGG
GTACAGCCTATCGAATCACTTACGCTTCCACTTTGAGATAACTTTTCGTTATTATTTATCAACGGC
ATTAACGATATCATTAACTGTTTGCAATGCATCGCTCAGTACACTAATTGGTGCTTGTTCAATATA
CTGCATGTGTCGTTGTACAAAATCAAAGGTGTGAAATTGTAACGGATTCACGTACCCTTCTATTTT
TTCAGTCTGAATCGGTACCATCAAGCCTGTTTCAGCTAAGCGATTATGTTTGGCATGCGTGATCG
GACAAACCAACGCTAATCCAGTCATTTTGGCATATTGTTGATTGCTAATAACGAGCGCTGGTCGT
CTTTTTTGAATTTCATGACCCCGACTTGGCATGAAATCAATACTCACAACATCACCTTTGCGTGGT
TGATAATGCCTAGTCCCACTCACTTGGTAATACCTCGTTTTCTAACGTAATATCTTGTTGGTGTAC
TTGTTGCTTGTACCAATCACCTTCAAATGGATTGCGGTGCTTCGGCAGATAAAGAATGCCACCAT
CATCACGTTGCTCAACTGTAAATTCAGTTCCATCGGCGATTTTAATACTCTTTGGAATGGTTAATG
TAATGGAATTGCCAACCCTTCTTGCTTTAACTGTCATTGAAATCATCCTTTCGTATACACCGAGTA
TACACCAGCGCGAATGAATCTGCAACTCTTGTGCCCTCTTGTGTACAAACACCACTGTCAATTTA
CTTTTGCCTATTGTGCTTTATCTCTTCTCGTTCTGTCATTAGTATGCCACCAACACGGCCGACTTC
ATCCGGCTCACCTTTGATGACGCCTTGAATACCATCGATTTGCTTCCAAGTCTTTACTTCCAAGTT
CTTCAAAGACTTTGATATCGTTTTTCGATGCCGGCAAACACTTCTTCATTCTGATGCCTTTCAAAG
AATTCCTCTAGATCATTCATCAGAGATTCTCCTCACTTAAACCTAGTTGGCGTCAAATTCCATCTC
AGCAATCGAATCTTCTAGGCTGTCCATTACTTCGTATGTTTCAATGAAGTTAACCCAGCTGTCATA
TGGATGTAGTTTGCTTTCTTTAATGCTAGTTCTAAGCTTTTCGTACACGTTGTCAATAAACTGCTCA
ACCATCCTATTAACCGCACTATTAATAACCGCTTCGTTCCACTGCTTAGATGCTGCACGCTTTCGA
TTTTGACTATGCTGATGGGACAGCGCCCGGCTTCTGAACAACGCTACTGCCCTGCTGTTAGCATT
CAGATAGGTTTCAAATTCTTCACGATCCATTACGTTTCCTCCTCAAAATAAGCCTCATTTCATAGC
ACAGCTTCAGCAAAAGGCATGTCATCCTACATGCCTTTTTTCTGTTGCTCTTCAATATCAGTATAA
AACGTCCTGCCGCTTTAGGCAAACGTATGTTCGCTATTAAGAACATACGTTTGTATAATAACTATA
AAAGATTTAAAGGAGGTCAATCGTATGGAAAACAATGTCCCGCGTGAAAAATGGCTTTACCCTGA
CCGCTGCATGAAGAAATGGCTGGGCTGGATTCTAAGCGACCATTCCGCCTATATGGAAGAAGCG
GCTATCTCAGAACAACCGGTGCTCCCAAAGCCTGAACAGACACAAGAAACCATTAATGGCGTACT
CGAAGATGCTTGGCAAAACTCAAAAATTGTCGCAGTTCAAATCGGTACGCCATACGATGATCTTC
TGTTACCGGATATTGAAGGCGCCGTGATTGGTCATTGGGACGCTCAGGTTTATCTACAGCTTAAA
ACTGGTGAGATGCAATCCATTAATGCAGCGGACATTCGCAATGTGCAACTGCTCAATCCAGATCG
GTGGTGGGCGTTAGTATGACGACACCATTAGATGATCCAACAAGGTTACCGGTACACGACATTAT
GTGCATTGACTGTAAGTCCTTTTACGCCTCAGTTGAAGCTATCAGACGCGGGATTCATCCGTTAG
CCGCCGACATTGCTGTTCTCAGCAAAGGTAATTCTCCTGGCGGTTTGGTGCTGGCTGCTAGTCC
CAACTGCAAAAAGCGTTACCACGTAGGACTGAGTACACGCCGTTTTCAGCTAAGGGACGATATG
TAGGTAGAACTTGCTGAACCGCGGATGGCTAATTACATTCGCAAGAATTACGGTATCAATCGTAT
TTACCGTCAGTTTACTGACGATGCTCACTGGTCTCCCTATTCCGTTGACGAGTCCTTTATTGACGT
TACCCACCCCCACAATCTCTTCGGTTCTAATGAAGAAATTGCTACCCAAATACAGAAGAAGGTGT
TTGATCAGTTTGGCATTGTCACAACAGTTGGCATTGGGCAAAATCCCCTATTGGCAAAATTAGCC
CTAGATAACGAGGCTAAGAAGTCAACGCCTTGGCAAGCCACTTGGACTTACGATCGTGTGCCAG
AAACAATATGGAAACTTGATGACTTGGTTGATTTTTGGTCGATTGGTAATCGAACTGCCAAGAAG
CTTAACGCGATTGGCCTTCATAATCTTTACGACTTGGCTCATGCAGACCGCGCCATTCTGCACCA
AAGATTCGGTGTTCTCGGTGATGCCATGTACTTTCACGCATGGGGTATTGATTACTCAGACTTAA
CCCGCCGCTACTTACCACGGGCCGAAAATAAAGGCTACGGCAATAGTCAGGTACTCATGCGTGA
TTACACTCAGGCAAGGGAGATTGAAGTCATGCTTAGCGAGATTGCTGATCAAGTGGCTGGCCGA
ATTCGCCATCACCAAGTCCAAGGTGAGGTCATTAGCGTTGGCATTGGTTATGCTGATGCAGAAGA
AGCTGGCACCTCCGGTTTCGGTGCGCAAATGAAGATTGATCCCACAAATCGCACAGACGATTTA
ATTCGCGCTACTCGATTTCTCTTCCATAGTAAGTGGAACGGACACGCTGTTAGAAATGTCTCAGT
TCGCGTTAATCGCATCAGCCAAGCAAGTACAATGCAACTTAGTCTATTTGAATCAGCAGAGAAGG
AGGAAGCAAACGCGGCTCCTATGCTGTAATTACGGATAAAAGAATCACCATCATTAGGTTTTTCG
TCTAACAATTTTAGGAAACTTCACTTTCTAGGTCGTAACTTTATTTTTGCAATCTAGGGTTTTTTAA
ATATATACATTTTAGCTCGTTTGTGTTTAATATTATAATCACAACTATACCAATGATAAATGTCTAAC
ATAAATATACAAACATGTTGACAGAAGCTCTTGAATACGTTTACAATTATTTCGTTCAGGCGAGCT
TTGTTTTTGAAAAAGTATTAATACAAGATAACTAGGTTAGTGGCTGTTGAATTAGGCCCCCGATTT
CGGGACCACGACAGTCACTTGATACTCGATTTTTATCGTTTGCTGGCTTGATCGTACATTGAACG
AAATTGGTACAGAAAAAAGAGCTAAGAGCCGCTCCAAATTAGCCAAAACGATTGCGGCGTCAATG
CTTACGGCGATCGTTTCTGCAGTTTTAGCTGTTACCAGATCAAGTCCTAGTTTCCCTTTGATGAAG
GCAAACTCACGCTCGATCTCACCTCGTCGATTTTCGGCTTGTCGGTCTGCCTTACGTTTGGCCG
GATCGACCTGCTTCGGCCGACGGCCCAATCTAGGACCGCTAAGTTTGATCCCAAGATCTGCGCA
CAGCCCGATATTCGCCCGAGTCCGATAAAGCGTATCAGCCAAGATCTCATCCGGGTATGTACCA
TACGTGTCAAAATAATGGTCGATCGTTGCTGGTAAGTCAGCACTTTCGTTAAACGCATTGAACGC
AAACCGTTCAACGGCCACGACGCCATGACTGATCGATACGTCGATCTTGGGCCCGAATTCGACC
GGATCCTTTGCTTTGCCGCGAATGATCGGTCGGATCGCTGGTTGATCAAGGCTTACGATCCGAT
CCGCGACTCGGTGAGTGTGCTGTCGATACATTTCAGTTTGTTGCTCATACAATTTTCGAATGATC
GTTAATCGTTGTGTCTGCCGTTGATTCAATTGCCCGCCTTGTGCTTGCAGTTCTTTGACGTAACG
CAAGTCACGTCGGATGTACTGTAATTGAGCCTTGATCTGCTTATGGGTCGTTTTCACCCAACGGC
GGGGTTTACGTGAAAAGGCGGTCCACGTTTGGTGGGCCTTGCGCTTATAGGTACGCGGCGGTTT
GACCGCTAATTGCTTGGCCATGGCTGCGATGAATCGCTCTAAATTGAGCCGCGCCTGATTGAGT
AGCTGCGTATCCTGCGGATACTTGATCTTTACTGGGACCGCAGTCGCATCAGTGATCAAGATCTT
CTGATGGCCAAGTTTAGCTTGGAGGCGATCGCGGACAAAATCGCTAATGATGTTCGTGATCAACT
CGGAAAGCGGCGCGATCCGGCGCCTGAAATAGGACAGCACCGAAAATGAAAACGGTGCTTGCG
GCTGATACTCTGGCAGGCCAATAAAATACTGATAAGCCGGTGTATCGCGGATCGCTGCGACTAA
CTCACGGTCCGATAGCTGAGTGCGCTGCTTGATCAGTTGGGCGCCATAAAGCAGCCGAAAGGG
TTTACCTGCCCATCCTAAGTTAGACGGGAAAGCCAATTGGTACGCCTCTTCTAGTTGCGGCCACG
GAACTTGGTCGGCCAGTTGGACCCACTCGTTATCTGGACTTAATGGGGTGCTTAAGCCGCTACC
AAACGATTTGATCGATAATTGAACGGCTTTTCGACGATAAACCATGATCCATGCCTCCGATAGGG
TCGTGTCAAATGCAAACGAAATGAGCACGATCCGTAAATTCATATGCATTCATTATACGACGATAA
CGGGTTCAACTCGCATCAAATGTGGTTATATCAAATTATTCAACAGCCACTAGGTTAAGATCTTCA
TTTAAGTGATATTCATTTGCAAGCAATTGAAAATTACTCATCACGAAGAGGATTTCATTGGCCATA
TTGGATAGCACGCAAATCACTTGCTTTAAGAAAATCAGTTCCTTTAATGAGTCTCTTAAAGGACGG
GGCTCTCACTTGTACTCACAATCAATGTTAACTGGAGATCAACAATATGGTCATAGATTCCCATAA
TAACATTGACTTGACTATCTAAAAGAGGCTTCTAACTTTGATATTGGTGGGGTTATTGGTTGCTTG
GCTGTAAGCAGATAATCTTAACTTGGGTTATTTTCATTGTGTTGTAAAGACATTTGTTATAAAGGC
CGAAGTTATCGCTTTGACTTGTAATAAATTATTTTTGATTGAGATATCAGAAAATAAACGGGGGAT
AATAATGAAAAAGATTATTAGGATTGTTCTTTGTGTTGTTAGTTGCGTTAGTATCATGGTCGGATC
GCTTGGGTTCTATTCAACTCCAAAGATCGTTAAAGCCGACAGTACATCTGTTACGGATGTCGACA
TTAATACCTATATTTCTAGCATGACACTTGATCAAAAAATTGGACAAATGTTTGTAGCACGAACCT
CACAAGATACTGATAAAGCTCGTGCTGATATAGCAAAATATAATCTTGGCGGGCTGATTGTTTATG
GTGTTGATTTCACTAGTGTTAAAGGGACAACAGCTACAGAAGCTCAGAATAACTTCAAGATGAAG
ATGCAAGGCTTTCAAAACTCGGCAAGTCTGCCACTATTGATTGGTGTTGATCAAGAAGGAGGGG
CAGTCTCACGCTTATCACAAAATCCTCTAATTGCCAACGGCAGAAGTTTTCCTTCACCACAAATG
GCTTATGCTAATGGTGGAATGACCAATGTAACAAAAGAAGCTAGTGAAGTCGGAACTATTCTAAA
AAATCTGGGCATTAACTGGAACTATGCACCAGTTGCCGACAGTACGCCTGACACCTCTAGTTTTA
TTTATGGTAGAACCTTTGGTCAAGATTACTTGGCTACTGCAAACTATATTACGAATGTGATCCCTG
CGTGGCAAAATGCTGGCATTGCCGCAACTCTCAAGCATTTCCCTGGTTATGGATCCGCGATTGAT
ACGCATACGGATTTTGCAGTCGTTACAAAGTCTAAGGAGGATTTTGAAAAAGAGGACTTGCTTCC
CTTTAAGTCCGGTATTACAGCAGGGGCAGATTCTGTAATGATTGCACATATAGTAATGCAAGCTG
TTGACCCAGTGTATCCAGCATCATTATCACGGAAGGTCGTTACCGATTTGTTGCGTAATGAACTT
GGGTATAATGGCTTAATAATTACCGATGCATTGGAAATGGGGGCCATCAAGCAATTTGCTCAAGA
ACATGATCAAGTTCCTGTTGATGTTCTTGCTGTTGAAGCAGGGAATGATTGCATCATGAATAACG
ATTATGAAACCGCTATTCCACAGATTCATGCAGCAGTAACTAATGGAACTATTAAGGAATCAGAAA
TCAATGAACACGTTTTCCGTATTCTTGATCTCAAACGCAAATTAGGGTTGTTAACTAAAGGACAAC
TTCAGCAAAAAAAAGTTCAGGTTGACAATGTTTCCTACAGCAGTGACAACAAAAAGGCAACTGTG
AGTGGAACAGTTGTTGATAGTGATTGGCAAGTTGGAGAACCATTATCGGTTAAAGACTCGACTGG
GAAGGTCATTATTACCGCAGACGTTGGTGCCGGTGGTAAGTTTACTTTCGATGTTCCTACTAAGT
CCCAAGAACAAGTATTAACTCTGACTACTAATTTACCCAACATCGCTGATTCTCAAATAACTATTAA
GGCTGTGAGTTCATCGAATACTAACAAAGCTTTGCTAGAAAACTTGATCAACGCTGCTGAACAGT
TGGATAGTAATCAATATACTGTCAAGTCGTGGGAAGAATTACAAACTAAACTAACTGAATCAAAAT
CGATTCTGAACAATGATAGTGCTACACAAGATCAAGTAGACGCTTCCGTTAATGCTCTACAAATTG
CCCTTAAGCAATTAGTTCCTGTATCAAATAGCGGAAATAATGGTCAAAGCTCTAATGATAGCAGTA
ACCAAAGTTCATCTAGCAGTAGTGGCAAAGAATCATCCAGCAATAGCAATGCCAATATTACTAGT
AAGGATCAGTCAGCTAAGGATTCAAATACGAGGCCTAAAGACCATAGTCTTTTGCCAAGTACAGG
TGAACGGGTGATGACGGGAATTTCTGTTCTAGGGGTAATTTTAATAGCTTGTGTGACTATATTATA
TATTCGGAAAAAAGGACGCAGCTTTTAATTAGTCTCTGCGTCAACTGGCGTTAAAAACTAGATTGA
AGTAATAAAGTTACCACCTGGAAAGAGGCATGCTCATTGCTTGCAAGGGTGTCGACGTGTAATAG
AAAAGTTGGGG
SEQ ID 12
Position 325750 . . . 327159
TGGCGTGGGCAACGTGCACGTTTTCTAGTCGCTAAACTGTGAACAATGCTCGTGCTAAATGCAAA
ACTGAGCAAGGAGATGAACTATAAGCGGGGGACCCTTTGCTATTGAGGAGGAAGGCGAAGTAGA
GAAAGAGCGGTGATTTGAACTCGAAACAGCGGCGCCGCAGGCTAGCAGCACTGTTAGATTAATC
GCCAGCAGCAGATACTAGACAGCTTCTTAAAGGCTTGATAATAGCGTTGCGCCATTTCAATGGAA
CTAGTGGTCAAAATCGCATTGTAGTTGCCATGACCCAAACTCGTTTTACGCGGGCCTTTTTGTAA
AATATATTGAACAACTTGGTTAATATGTTCATCGGTCTCAAAGTCAGCCGGAGTTAAATACGTTTC
TTCCAAGTCCTTCACTGACATCGCTTGAATCTTGGCTTGAATTTTCGCTTCATCAGCCGTACTCAG
CAGGCGGCCTTGCTTATCACGACGAGCTTTAACGCGCTGAGTTTCTTTTTCAAGGGCCCGGGTT
ATTAAAGCGTCTTTACCAATCGTTGTCACGTGTTCCACATTAAATGGTAACACTGCTTGGTCCTCT
AAGGCGTCTCGCAAGTTATAAACATGACAGACTTTACCAAATAGCTCCTCCGTCGTAACTGCTAG
ATCACCTTTGAGCTGTTTCTTATTTTCATTAAAAATGGGGGTGCCAGTGTAACCATACCAGTTACT
ATTGATAAACGCTGCTCGAATTTCCTTTTGCATCTTACCAAACTGCGACCGGTGGACTTCTTCAAC
AAAGAAGATCACCCGTTGCTTTAAAGTCTTACTAAAGCGGGATTGCTTACCGGTTGCCAGCTGGA
CTTGCGTTTTTTTGACCGCCCGATGGAGCTTTTGAATCGAGGTGACCAAGACCTTACCGTCATTT
TGTTGCAATTTACGCATTAAATCACCGGTGTTTTGGGCTTCGTTAATGGCAATATCATCATTGGCA
GCATAGGCACTAAAGTTGCTGGTTGTCTGTTCGTCTAAATCCCGCCGGTCAACTAAGAAGATGAC
CTTATCGACACCAGGATCTTGCGCAGCTAATTTAGCGGTTTTATATGAGGTGAGTGTTTTACCAG
AACCCGTGGTATGCCAAACGAAACCATCCTGATGGTCATGAATCCGGTGCATCACGGCTTCAAT
CGCATAAATCTGGTAAGGCCGTAAGAGAATTAAGCTTTGCCGCTCTTGGTCGATGACTGTATATT
CACTGACCATTTTGTGGGCCATGGGAATATTAAGGACTTGGCGCGTGAACGCTAACCCGTTTTCC
ACGGGGTGATTATCCCGCGTCCGCCAATTGAACAAAAAGGCTTTATTGAAATGATCCGGTTCGG
CATTCGCAAAATACGCCGTACTATCCGGCGTCATAATCACAAAC
SEQ ID 13
Position 328723 . . . 329314
CGATCTAAAAGCTAAGTTATTTTCCAAGATATCAAACAACTTCTTAACCCAAGAATCTTCCACACAT
AGGACAATAATGAATCCAAATAGATTCAGCTTTTTCCTGCAAACCGGGATCAGTATAAACGTCCA
GTACCGGATAATCACGCATTAAGTTCAGCTGCCAATGGGTATCATCTAAATTAAAAAGATCCGATT
TAGTGTCTCCCCTTACTACATTATGGCAATACACACAACTGTTGTTATACATGCTTCCTTGCTTTTT
GATTTTAAACTCCTCCATTTTGCATATTATAAGAAGATTACTTCTACTTGATATATAGATGCTTTCC
TTGCGAGGGTAAGTCAGACAAGGAAGCATTTCTAACTTGAGATACTTAAGCTTGTCTCAATAGAT
GTAGATAGCGGCTCCCCAATCGGATATTAACAGCTCAACTAGTCAAACCAGATATATAAATGTGA
CACAAGCTGGAATATATATCATTATCTAGATAATTCAAATTGAGCTAATAAAATCAATAAAGAAAAT
TTTAAATAACATTATTTTATAAACCCCTTTAGGATTTTCCCGATTTGATATTCTACGTATGTT
SEQ ID 14
Position 2002858 . . . 2005090
GAGTATCCAAAAATACGACGGGTATTTGAATAGGATACTTATTAAGCGAGAATGGTATTGGAAAT
CTGTGGCAGCCACTCAGCGGAACCATACCTTTATCCCAACCCCACGCAAAAAAAACATCAAGTAA
TCCGTCAGATATGATGACTTAATTGTGGGACAGTTCTAATATGAAGAAAACAGGTTAGATAATTGG
GGTGAAAAGATGGCAACCTATTCGCAGATAGAACTAGACATAATCAAATCATTTAAAGGGCTGAT
GAAAGACCATGAATTCACTGAGATCTCAATTAAAATGATCGCTGAAAAAGCCGATATCACTCGAC
GCGGCTTTTACAATCACTTCTTAGATAAATATGATCTTGTCAGTACCATCTTTGAGCATGATCTTTT
TCCAACAGTCATCAGTTTGACGAATATCAATGACTGGGATCAAGGGTCGCTGTTTATCGTGAATT
ATCTCCAAGACAATCGCGACTACTATAAAAAATTGTTGTCGCTTGAAGGACAAAACTGTTTACAGA
CAGACTTTTATAAATTGACTGAGATGCAGATTGGGATCTTGATCCCAGAAATATTGGTCGGTAGG
AAAATTTCTGACGAAGATCAGGCATTTTTAAGCGATTATTATTTTCACGCTTATATGGGACTGACT
ACCGAATGGGTCAAAGGTAAATATGGTTTTTCAACTCAGGAGTTCGTTAAACGGTGGAAAGCCTT
ACTCAATAATTCAATGCATAATTATCTGGACAACTACGCTCGATGAATTACACAGATTGGATTAAA
TGAGAAAGATGTTACATTTGTGCCAATATGTGAATTGATAAATATTTCACAAGGAACTATTCTTTCC
CTGTAAACGAAAGTTGACTTGAAAGGAGTTAGTTCTGATGAGTAAATATAAAGTTATTATTTGGGG
ATTAGGAAACGTTGGTCGTTCCGCAGTGAGAATGATCGCGGAAAGACAAAATATTTTTGAATTGG
TTGCAGCCGTTGACGTTGATCCAAAGAAGTTAGGTAAGGATGCCGGAGAAGTCTTTGATTTTGAC
AAAGTCGGCGTCAAAGTTTCAGATGATATTGATGCAGCCTTGAAACTTCCAGCTGACATTGTGCT
CGACTTTTGCCCAACGGAAATGGACAAACAAGGAACATTCATGCCTTCTGCTATTCGACTCGCCA
AATCGCTCGATGCCGGTAAAAACGTTATTACCACGATTCCGGTATATCATGTTCAAGACAGTCAG
CCAGAAGTATATGAATATCTAAATGAACATGCTAAAGCACATAATGTTGCTTTTGTACCATTTGGA
CTTTTGCCAGGCGATTATGCCTCATATATCCCACTAGTTTTGGCCGGGGCCATGGGCCACGTGG
ATAAAATTGTTGTTCAATCCGGTGAAGATGACTGGCACAACACATCAGGCTGGGTCGATGTCTTC
TCATATGGCGGCGATATCAATAAATATCCAAAACCAGACTCAGACGAAGATCTCTTGGCTAAGTT
CATTTATGCTTATTATTCATCCGGCGTATACGAGATGGCCGATAGGATCGGTCTGAAATATGATA
CCTTCAAACCAGAGCATGAAGTCTTCACTGCACCCAAAGATTTGGAAACGATCAAGGGTACAGTC
AAAAAGGGCAGCATTTATGCCCACAGATTTACCATGGCACTTTACAACGGCAACGAACAAGTAGC
CGCCTTAAGATATGTTCATAAAGTTGATAATAAAGAGACACCAGAATTACCGATCAATAATACGAT
TCATATTGAAGGCTTGCCGTCAGTCGATGCGCAGATCGATGGATTGATCCCAGAAAGAGAAGGC
TACGTTTCATCAGCCGCTCCAGCAGTCAACTTGATCCCTAGCATTCTCGAGACCGACAAGACAG
GTTATGTTGAAGTCTGCGACCTTCCAGTAGTGATTGCCAGGCCATTGGATATTGGCGCAAAAAAA
TTAGTCTAGACTAGGCTTTCGAAGCTGCTTTGACCATTAAGGTTGGAGTAGCTTTTTCATTTGCAA
GTAAATCATTACGGCTTGTGTATACGGTATACAAAATGGAGAAAACGCTGACTAGTTTATAAATCA
TTGAGACTTAACGGCCGGATAAATGCTGATCTGATTATAGAAATAACAACAAAAAGGCCACGCTA
AAAATCATATTAATTATAATCGGGAAATTTATTAATAATATTCAAGAAAAATAAAAACCGTGGGTAC
ATTATTTAAAA
SEQ ID 15
Position 2262750 . . . 2268615
TTTGAAACTAAGACGAAAGCTGCCATGTCAAACAAAGCCGCCATAAATGCCACTGTCACA
GATCCATCAGCCGCAATGCCAGCATCTTGCTGAAGTTCTTTAACAGCATTAAGGGTGTTA
TTCGTGAACTCATTATTAAAGTCTACTGGACTAATCCCTTTGCACCAGAAAGCCCCTTGA
ATGAGTTGAGCAATGTTCCCCTTATATCCTGGCTTCAGACTACCTACAACAGGTGCTAAG
GCGTTTTTGGTCGTCTCGCCAAAGCCTTCACCAATAGCACTAATACCGATTTCGTGCTGT
AATCCCATTCTTAGGCTATAAATTGTTGGCCATCCCGTTTGCCCGTTTTCTGGAGCTGCG
ACAAAGCCAGGAACGCTACCATACGTTTTGTTGAGCCATTTTTGAACGGCTCGTACTGCT
TCATCTGCCATTTTAAAGTCTCCTTTTTTGTTTTAGACAGCACGTCTGCCGTCACAAAAA
GCAAACATATGTTCGGATTCATTTCATCTCTTCAAAGCTTCGAAAGGCAACCCTGATCCA
CAAATAATCCTTTTATTTTGAACTTAGCAAAAAAATGAGGCCCTCACATAGTGTTGAAGT
TGCCTCATTCTTAATGTCTATATTTAAAGTATTGCCACAACGATGGATCATCGAACGCTC
ATGGACTTGGTTAGACATGTATCGACGACTATGAAAATGTGGGCGCAAGCTCAATTTCAC
CTTCCAAATGTTTGTGCTATCTCATTTAGCGCTGGTTTTTTTAGGATAGACTAGACAAGG
ACTAATAATTTCTCAAGAATCCCGCAACTCCACTATTCATTCGTCGAAATCCCCACTGAT
ACTCTTGTCCTTGCACGTTCGACCAAGCAAGAATGTTTATTCCGATAACCGAATTGTTGC
CATCAAGTAATGGACCTCCCGACATGCCATGATACGAATTAATTTGTTGTGAAATATATA
TTCCTAGTGGGTCCTCTGAAAAGGGTGTGACAGTCCCACTTGATTGAACCATGACTCCTT
GAAGTTCGTACCCTGATTGCGGATCGCCAGGGAATCCAATGGATCTTGCTGCCATCGTAT
CAGCAGGGTTCGTATTTAAATTAAGACCCGCAGGCATACTACCAGACTTCATAGAGACAA
TTGCAGCCCCGTAATCATTCGAAGTAGCTGTTGAATTATTAATCCATGCCTGTGGCACTA
TCAATCTATTCAATACTCCGTAACCGACCCCTTGATGATTTGCTTGACTATCACCAAAGT
TAATAATTCCTCCAGAAATATAATGACCATCATATAACATGTGTGCTGCTGTCCCTATAC
GGTCTACTCCAATGCTAAATCCAGTACCGCCAGAAGTTCCACTGCTCAGCTCTGTGCCAT
TTGAGTTAGAGATGATTTTTTTATACGAACTGTCAAGGTATGGTGAGTTAATGACCATGA
CACCATTTGCCATCGAAAACCACGTGCTCAAAACCCCAACGGAACTGTACGGCGCCGAAT
TGGGGTTTGACACAGGTGATACCGTCCTCACAGATAGATGATTAGACGATTTATTCAGTT
TCGCAAGATATTCGGACGTAATTCCTTGAACTTTCTTTCCTTCTTTTAAATCCTGATATT
GATCAGGTGTATAAGCTTTGACACTCCCTTTAAAATCGGGATAAGAATACTGATATTGTC
GAATGATTTCATTCAAAAACTGTTGTGTTGTCGTCTGGTTAGTCAAAACAATATCATTCT
TTGGTAAAACATGGCTATTCGCTGCCAAAGGATTTGCCATACTATCAGCACTTACACTTA
CCGTTTGAACTTGAATTATCGCTAAGGCTGCAGCTATCATAGTGATATATGCCCATACTT
TTCGCAATTTAATTCCCCCTTTTTCTTAAAATGAAACCGCATTCACGGAGGCTTGTCAAT
GCTTTTAAAAAACAAACGTTACTTTTGGCTCATCTTGGCTGTCAGCATAATTGGAGTAAT
TGTTCTTGCGGTGTTATGGCGCATGAACCCTGAAGGAACGGCCTCAAATAAGTTTGAACG
TCCCACCATTACTATTAAAAAAGTCAAACTTATTAAGCACAGTAACAGTATTGCTGTCAC
ATTTGCTACCTCTCCAAAAAGCAAATATACGATAAGTGATCTTAAAGAGAATCAACTTTC
TTCTGGCATTTCGAATAAAAGAGAAAATACCGTTTCGGAATTAAAGCCCTCCTCCTCTAA
GCTTGCAATACGGGTTAAGCATAACAATAATATACAAACCAAAGTGGTTTCTGTTCCCAT
TGGTTATCATATTATGAAAAGTGCCATTTCAAGAAAGCCAATTCCTATGGGAGAAGAGTT
TAAGTACAATGGAAAGTCGCATGTTTTATTTAGCATGACCATTACCCCTAAAAAACAAAA
CAAGAACAGTATAAAAAACACCACTGCTTTTAACATAACGGTTAAAAATGATCACTACCT
TGTCCCTGTCGTATTAGATACCAAATACCTTACTGTTTCTGATTCAGAAGGTAACTCATT
AAAAGTAAAGCCATTCTCAAAAATTTCTATTCCAGCAAAAAAGAAAAAGACCATTGCAAT
AACTATTGAGGGCGTTCCCGCAAGCTCTGCCAATGGTCTAGTTATAACGTATAATACTGT
CGATTTAGACTTACCAATCTCCTTTATAAATTCCTGAAATTACACTAACTGTCCCCCACC
TTGACAGTCAGTACACTCAAACTGTCTCTTATGCTTACAAACACGTAATTTAGGCGGTTT
TTAAGCAAAAGTCGTTAGTTTTCATAAATGTTATCTTATACTCTAATGAGATCTAGCTTG
TGATAATAAGGCTGTTTTTCTTTGACAGCCTTATTAAGCACACTAATCAATGTCAATTCG
AAGTTTTTGGTTTCCTACTTGGCCAACTTTGTTATCAGAAATTCCAAAACTCATTGCCTC
CCGCCACCATATATTTATCGAGCCATTTTGAAAATGAAAAATCGAAATATCGGTCTGCTT
CTATTCCGGGATGAGTTAGATATGATTTTCCTAACCGATACTTCTCTATATCAATATACA
TATCTCCGACATCCCTTATATGGAGAATGGGTACTTTATTTACAAAACTTTTAGGCAGTG
CCTCTTTTTCTTTAATCATTTTTCTAATTGAATAGACTTCACACGTATATCCCATAGGAA
TCCCTTCGATTGTTGTGTCAAATAAAAATAGTCCATTAGTAATCGAGAGAAACTCAATGT
AATCCTGTGGAAGGTTCCACCTTTTTATTTTTTCTATATCATCAGCGTGTGCAGGAGGTT
CTATCTTAAAAGAAACATTTTGCACATCTCCATCTAATTGGAATGTTGAGAGCGCTTTTT
CCCCATTTTTCGTCACCTTTATTAAAGAATTAATTCTTCGCCGAATTAGAGATTCCAAAT
GAGTTCCTCCTCAATAGTTGTTAAACCACGCGGTGATCAACCGATGATTTGGTGTTAACA
CCGGCATCAAATTATTAAAGTCATTTGTTCCGCCATAAACTCTCGGGCGAATATGATGCA
CTTCTCGAGAACTCCAAAAATCTGCAGATTGATTGCCATATGTTTCACTGAACGTTTTAA
TATAAATATATCTATCTTTTGATGACCAACTAGGAGATTTTGATAACTTAGTCCAGGTAG
TATTAATAGGTGTTTCTGCATCTTTTTTAGACACGGGATCAACATATTCAGGGAAATTCT
GTCCTATTTTATTTTGTAAATAGATGGCCGTTGGTGGGATTGGTTTTACATTAGCCGCTC
CATTAACTCCTATAAATCCAACAGCGCCAGCAACGTTAAAAAATGTGGTTTTGGCTGGCA
ATTCTGTAAAACCAAGTAATCCTGTTTGCAAACCGCCTATTTTGCTAATGGGTGCCAGCG
TATTGGCATTCACCGGACCTGCAAGGGTAGGCTCAGTATTAAATTCAATTTGCACATCAA
CTGCGGCTGGCGGGACACCCTCAATAGAATCAATCCAGAAATTGACTCTGAATTTTCCGG
ATATGTATTCTTCCGATAAGTGCCAGGTAATATTTGTAACAGGAACTTCTGCACGAGAAC
TTATGCCATTGCCATGACTATTATTCAGTACCACTTGTTTTCCAGAAGCGATAATTGGTT
GTCCATTTTCTGGATTACTTCTTGATAACTTGTCAGCATTGGTCAGTTGAGTATTGTTAT
CGCTAATACGGCTTTCAGTTGAAACAATTTGATTTAAAGATTGTGTTGTGTCTGCGGATA
CAATTGTTGAATACCCAAGCAAATTGACTAGAAAGAGCCCGAATATTAGCCCAATAACTT
TCCACTTTTTCACGCCTATTATCTTCTTTCCAAAGTTCTTCAGTGCCTGGCAATAACTGT
ATACATTGAGCAGTATAGTCGCTATTTTATAGCTGAACAACTCATAAAGCTCAATTATTA
TTAGCCTATAAAACCACTGCCTAAGTGAATTGATCTAGAACGAAGCACGCCGAAGAAGTC
GCTAAATGTGCTAAGAAAAATGTGCTTGAATAGCTCAAAAGTAATTAGCGTCTCCATTGA
AAATCCGTTATTTTTAAGTGATCTAGTGTTAACTATGAATCCCAAATAAAAAGCAAAATC
CGTAAATGCCAAATTTTCCTTTTTGACGTTTTTCTACTGTCGCGAGATTTGCAAGTGTAC
GTACACTTACGATGAATTGACAGAATCTCAGCTGCGCTGATCGTCAATTTTGTTTGGGGG
CACGCCCCCAATCCCCCTGTTATTTTGAAGGGAGGTGAGTCCCCCTTCAAAATCAAAATT
TAAACAGCATCTGCCGCCATCTTTTCGCTGACCTTCTCACGATGTTACACGTGGTGTTGA
CACCCACTTGCATTTAGAGTTTCATTCAAGTTGAACATTGTGTAATATATGAGTTGCATT
TGATAAACATATCAGTTGCTATTTGTGCAACTTTAAAGCTTCGGCTAATTCAACGTTCTG
TTAATTTACAAGCATCTCGACAGTTTCTGTTAAAGCAACATCTACGCTTCAATTCGAGCA
ACTCACTATACGTATGCCGAGTTGCAGACAAGCTACTATATAGCTGTACGCGCTGAAACA
CCAAAAATCGTTCGTTTATGCCCAATAAGCGAATAATCTTGCTCAGGTGTAGTAAAAAAC
TGTTTACGTGTAGTGAATGGCGCTAGCCCTTGTCGTAACTGGCATCATCCACGTGTAGTA
AAACGCGTTTTACTACACGTTCGTAATTTTTTCACGTGGAGTAAATGGCGTTTTACTACA
CCTTTTGACCCCAACGTGCTATCACGACAAACCAAACCGCACTGCGGTTTACCCCAATTT
TGGGGTCAGTTTTGCCTTATGCTCTTTCATGATTTTAGGCGCGTTCCAAGCAGTCTCAAA
AAGTGGTCGATCCAGGCGAGCCGATTTTTGAGAAGGATTGGATAGCAACTCAATTTATTT
TGATCTTTTGCTTGGAGAAAAACGTTCACGTTTTGACCAGGGCCGTCGCAACTGTTGACC
AAAACTCGTCCGGTAACGTGACGCTATTTAAACGCCGCGTTGGTTTGCTAGACGACCATT
CATCATCACCATTCAGGAGGTTTTTGAAATGACAAAGCAAGACGAAACACACCGGGTCAT
GTTCACTTTGACCGATCAGGCGATTGCAAAATTGAATCAGCTGGTCGCAAAAAAGCAACA
GGAAGTGAATCAAAATCCGGAACTGGCTAAGTACCATGTCAGCGTGACCAAATCAAATAT
CATTGAGGACTGGTTATCAAAGCAGTGAGTTTAAAAAGCGCTAAAGGGCCTGTACTAGCG
TTTCTTACTCTGGTGGGTATAATTAATGCTCTCTACATCAAAAACG
SEQ ID 15
Position 2262750 . . . 2268615
TTTGAAACTAAGACGAAAGCTGCCATGTCAAACAAAGCCGCCATAAATGCCACTGTCACA
GATCCATCAGCCGCAATGCCAGCATCTTGCTGAAGTTCTTTAACAGCATTAAGGGTGTTA
TTCGTGAACTCATTATTAAAGTCTACTGGACTAATCCCTTTGCACCAGAAAGCCCCTTGA
ATGAGTTGAGCAATGTTCCCCTTATATCCTGGCTTCAGACTACCTACAACAGGTGCTAAG
GCGTTTTTGGTCGTCTCGCCAAAGCCTTCACCAATAGCACTAATACCGATTTCGTGCTGT
AATCCCATTCTTAGGCTATAAATTGTTGGCCATCCCGTTTGCCCGTTTTCTGGAGCTGCG
ACAAAGCCAGGAACGCTACCATACGTTTTGTTGAGCCATTTTTGAACGGCTCGTACTGCT
TCATCTGCCATTTTAAAGTCTCCTTTTTTGTTTTAGACAGCACGTCTGCCGTCACAAAAA
GCAAACATATGTTCGGATTCATTTCATCTCTTCAAAGCTTCGAAAGGCAACCCTGATCCA
CAAATAATCCTTTTATTTTGAACTTAGCAAAAAAATGAGGCCCTCACATAGTGTTGAAGT
TGCCTCATTCTTAATGTCTATATTTAAAGTATTGCCACAACGATGGATCATCGAACGCTC
SEQ ID 16
Position 2776965 . . . 2787971
GCCACGAACCTGTAGCCGTTTGGATGAAGCCATATAATACTGGACCAACCGCCGCAAATA
AGTAGCCGACACTTTGAGCAAAATCAGGAATACTAATCTACTTTGCCCTTAAAAAATCTT
GAGATGATCCATATCTTGTTTTGCCTTCATTACTGTAGTTGGTCATAAGAAGTGCCCTAC
ATTCATTAGATTACTTGTCTAATAATTGTAGGGCACTTGGGTTGAGAAAAATGATGTTAA
CTAAGAATGCAAACGAACTAAAATCTTTGCTTGCTTTTTATCCTTTTCTAAGGATTCAAT
TCCTTCTGAAACTAATTCATTTAATTCAATCTTTTTTGTAATGACCTGTTTGAATAGTGA
ACGATGGGTATCTATAATCTTAATTACTCGATCGAAGATATTGGCATATCCATAAGATGT
TAATAAACTACCACCTTTTTTAAGAAGAGCTCTAACATCTACAACTGGTGGATGTTGAAA
TAATGCAATCACGGTAACCTTGCCACCATTTTTAAGAGCCTGAATGGCACCAGTAAGTGT
GGGTTGTACACCGGCGCAATCAAACGCAATATCCACTCCCTGATTTTCCGTGATAGTGCT
GATAGCGTGAGCTAATGACTTTTGACTATCAGCACGTATTGGGTATTGAATTCCTAATTC
ATTTGCTAAATTCAAACGTTCCTCTGACATGTCATTTATTATGACGTGATGTGCACCAGA
AATTTGTGCTATTAAGGCCGTGAACAATCCAATTGGACCAGCACCTTGAATTAAAACATC
ATCTCCAGGAGACACTCGGCTTGCCATAACTGCCTGTGCAGCAACTGAAACTGGTTCAAC
TAAGGCCCCTAAATCAAGCGGAAAGCTAGCTGGTAAGAGATGTGCAAAGGTACTTTTTAC
ATTGCACTTTTCAGCTAAGCCACCGTTAGCCGAAAATCCTAAGAATCCTGCTGATTGATC
ACTACCTATAGCATGTTCACACCAATTATAATGACCAGAAAGACATTCCGGACATTTTCC
ACAAGCAATCATTGGTTCGACTGCAACTTTATCTCCAATTCTTAATTTAGATACTTGTTT
TCCAATTTTAGAAATCGTCCCAGAAAATTCATGACCAGGAATTAGCGGGGCTTGCATATG
GGTTAGCGGATGAGGTATTGTCGCCAAATCCATACCCTCTAAATATTCATGAATGTCACT
ACCGCAAATACCATTAAATGCAACCTCAATTTGAACTTCATCTGGTGCGGGATCAGGAAT
ATTTCTTTTTTCAAAGCGGATATCCTTAGGACCGTAAATAACAGCTGCCTTCACCATAGT
CATAGTGCTTCGCTTCCTTCATGTTCAATATAGCACAATCGTATATAAAATAGTGAATAG
ATTTCAGTAATGAAGTTACCATCTTGACTTAACAAAAACTTGCTAACTGATTATATGAGA
AACTTTTACTTGAAACATTTTTGGTGATTACCATTAATTCCCTCGGACATATTTTGAAAA
ACCCTATTTGATGCTGATTGCAAATTATTTTATGCGTATTTATTAAGGGTTTCTATGTTG
AAGTATATAGCAAACTTGTTCAAGTAACTGACTTTCACGTGGGCTTTAGCCAAGAGATGC
TGAGCAGCGAACCCAAGGGGTGTTACTCGCCCACGCAAAAAAGAAATCCAATTGCATTCC
AGTATGAGCGAGAAGCAAGCCATTAAGACGCTGATTCATGAACTCGCGCACAGTGAATTA
CATTGTGATCCGAAGTTAAAATTGGATCGTTCAACCATGGAATTGGAAGCTGAAAGTACC
GCGTTTATCGTTTGTCAACATTTGGGAATTGACACGAGTGATTATACGTTTCCTTACCTT
GCTGTTTGGTCGAAAGATAAGGATCTTTCCCAGCTCTCCAAAAGCTTAACGCGTATCCAA
TCCACCGTCGAAAAATTCAATAAAACCGTCGATCAAAACCTTGAAAAGATTCGTGAGAAA
CCGTTGACGCTTGATCAAAAAATAGAACGCGCTAAAACCATTGCGACAACGGAAAACATC
GCAAAAAAAGAGCAAGGGCTGGTGCAAGCAACGCAGGAGAAAACACGCTAACCCATTTGT
TGAATACTCTCACTCAAGAGGACACTCCAGCCCTTGATCACCCAAGAAAGGAATTACCAA
CATGAAAACCATTGACGAAATGAACGAATTCGATCGTGACATTATCTTACTTCACCGCAA
GTCTGTGAGCGAAGATACACCGCAGGCAATTCTTGTGAAAGTGAAACAGATTCGTAACGC
AATTGCCGACGAAAAGGCGGGTAAAGAAGATCCAATTGAGAAAGAATTTACACTCGAATG
TTACGACGAAGCAATCAGAAAACTAAGGGACCTTTCGGTCGCTGATTATCAGTTGTGGTT
GCGTCAAAACAAAGACCTGGAAGGGTTTGAATTTTGATTTTGAAGGGTGTCGTAGACCCC
TTCAAAATAACGGGGGATTGGGGGCGTGCCCCCAAAACAAAATTGACTATCAGCGGAGCT
GATATTGGATCAATTTATCGCAAGTGGACGTCCACTTGCAAATCTCGCGACAGTAGAAAA
AGCCCCAGAAAAGCAAATCTGAAAAAATGTAACAGGCACTTGATATCAAGTGCCTTATTG
TTTCTAGGATCGCTAAAAATAACAGGAGGTGGTTACATGAAGCAATCTGATGAACACCGC
ACGCGTTCAGTGAGAAGCACTGTGCGTATGACCCCAGAGGAGCGTGCTTGGGTTGATATG
AGAAGAGCCTCTGTCGGCAATCCAAAGTTCAATGCATTTGCCTGTCGCGCACTCACGACG
AGCAAGATCGTTCATGTACATTTTACTGATACTAAAAAGTTACTTAGACAGCTGTCAAGA
ATTGGGAATAAGGCTCCTATGCTGTAATTACGGACAAAAATAGTTTGTGCGATAATTACA
GCATAAGGGCCTCTAGGTCGGAGCCCAGGAGGCGGAGACCGCCGCACAGCCCAACCCCAC
GCCGAACCGGAGGCCAGCCCGCCCGCACCGCGGCCGCAATCATCCACCCAACGCCCCCCA
AGTTTTTGATAGCGGTAACAACGCCTGTGCGCTTGTCGTGGCCGGCCTTTTTTCATAAGG
TTGGAGGAGAAAGGAAGGGTGGTTATGGGCGCTTGGTATGAACACGCAATTATTTACCAA
ATCTATCCAAAATCGTTTCAAGACAGCAACGGCGACGGCATCGGGGACCTGAACGGGATC
CGGCAACGGATCCCGTACCTGCAAGCCCTCGGCATCAACACGGTGTGGCTGAACCCGATC
TTCGTCTCCCCACAGGTGGATAACGGCTACGATGTTGCCAATTACTTCGCCGTGGACGAA
ACCATGGGTACGATGGCCGACCTGGAGGCGCTGATCGCGGCTCTGCACGCGGCCGGCATC
CGTCTGATCATGGACTTTGTGCTAAACCACACCTCGGATCAGCACCCGTGGTTCCAGGAC
GCCATTCACGCCAAAAATAGTCTGTACCGCGACTACTACATTTTCTCTGGCCACGACGGG
CAGCTGCCAAACAACTGGGGCAGCTTCTTCGGCGGATCGGTTTGGGCGCCGGATCCGGCG
GGAACCGGGCAGTCGTATTTTCATCTGTTCGACCGGCGGATGCCGGATCTGAACTGGGCC
AATCCCGAGGTGCGGCGGGCGATGGGAGACGTCGCCACGTTCTGGCTCGGCAAGGGCATC
GACGGACTGCGGCTGGATGCCTTCATCCACATTGCCAAGGCCGATCTGGGGCAGGATTAC
CCCCTGGCTCCGGGGCAGCAGACGCCGGTGGTGGCGGAGCCGTTTTTCTCCAACCTCCCG
AAGGTGCAGGAATGGCTGCGGCCGTTCTGCGACCGGATCAAAACCGACTACCCCGACGCG
TTTCTGCTCGGCGAGGCGGCATCGGCCAACGTTAACCTGGCGGCGGATTACACCGCGCCT
AGCCAGCACCTGATGGACAGCGTGATCACGTTCCGCTACTTCACCGAGGACGAAAGCGGC
CTGGATCCGCGGCTGCCGGCGCAGTACCAGCCGCGGACGCTGGATTTCCCGGCGTTCAAG
CAAACCCAGGCGGTGTGGCAGCAGACCCTCGCCGGGGTGTCGATGCCGACGCTGTACTGG
GGCAACCACGACATGGCCCGGCTGGCGACGCGGGTGGCCAAAACCACCACCCAGGCGCGC
AGTCTGGCCATGCTGATGTACCTGCAGCGCGGCCTGCCGGTGATCTACTATGGCGAGGAG
CTCGGGCTACACAACCTGCAGTTCGATCACGTTGATCAGTTTGCGGACGTTTCGGTGGCG
CCGTTCGTGGCCGCGGTCGAGGCCACCGGGCAGTCGCGGAGCGCGGCGCTGGCCATGGTG
TCGGCGACGCACAAACTGCCGGCACGGGGGCCGATGCCTTGGACGACCGGGTTGCACCAG
GGCTTTTCCAATCACCTGCCGTGGCTGGTTGGGCGCAGCGAGGACGTGACCAGCGTGGCC
GCGCAGCAGGCCGATGAGGCCAGCATGCTGCACTTCTACCAAGCGCTGATTGCCCTGAAG
AAGCAGCCGCTGTTTCAGGCCGGGCATTACCGGCTGCTGACGACGGCGCCGAACCTGTAC
GTCTACGAACGCACGCTGGCCAGCCGGCGGGCCCTGGTGGCGGTGGCCTTGGATGAGCAA
GGCGCCACCTTCACCGTGCCTGAAGGCCTGACGACCGTGGCGCTGGCCGCCGGCGATTAC
CAACTCGAAGGTCAAACGCTCACGCTTGGCGCGAACGCCGGCGTGGTGTTAAACGAAAGG
GGAACTCGATAACCATGCAACTTGCAGCATTACGGCACCGCCCAGAAAGCGAAGATTGTT
TTTTGTACACTCCAGATGAGCTGCGGCTGCGGCTCCACACAGCCAAGGCCGACGTGCAGG
CGGTCATCGTACTGTACGGGGATCCGTATGTCACCGCGCCGAACCCGACCACCGGAGAAC
CGGAATTCGCCTACCAAGAGGCGGCGATGATCAAAACCGGCACCGGCCAAACCAGCGACT
ACTGGACCATCAGCCTGACCGCGCCTTATCACCGCCTGCAGTACCAGTTCCTGGTGACCG
GTCAGGACGGCAACACCGTCCTGCTCGGCGACCGCGGCTTGCGGGCCGACAGCGCCGCCA
ACCGCCGGGCCGATCTGTTCCGGGTGCCGTACTTCCACGCCATCGACACGGTACAGACGC
CGGCCTGGGTCAAGGAAACCGTGTGGTACCAGATATTCCCGGAACGCTTCGCCAACGGGG
ACAAGACGAACGACCCCAAGGGCACCAAGCCTTGGCGTCCGGCGGATCACCCGGGCCGTG
AGGATTACTACGGTGGCGACTTGCAAGGGGTGCTGGACCACCTGGACGACCTGCAGGCGC
TCGGCGTGAACGGGCTGTACTTCTGCCCGGTGTTCACGGCGATGTCGAATCACAAGTACG
ACACCATCGACTACTTCAACATCGACCCTGCGTTTGGCGACAAGGCCTTGTTCGCCGATC
TGGTCAACCAAGCGCACCGCCGCGGCATGCGGGTGATGCTGGACGCTGTGTTCAACCACA
TGGGCAGCCGCAGCATGCAGTGGCAAGACGTGCTGAAGTTCGGTCCGCAGTCGCGCTTCG
CCTCCTGGTTCCACATCAACCGTTTTCCGGCGGCGCCCTTCGCCGCGCCGGAACAGGGCG
GCGTGCCGCAGTACGACACCTTCGCCTTCGAACCGCACATGCCGAAGCTCGACACCAGCA
ACCCGGCGGTGCAGGACTACCTGCTTGAGGTGGCGACGTACTGGATCAAACAGTTCGACA
TCGACGCCTGGCGGCTGGATGTGGCCAACGAGGTGGACCATCACTTCTGGAAACGGTTCA
ATCAGGCAACCAAAGCGCTCAAGCCCGATTTCTTCGTGCTGGGCGAGGTCTGGCACTCCA
GCCAGCCGTGGCTTAACGGGGATGAGTTCGATGGGGTCATGAACTACGCGTTCACCGAGC
AGATCGAGGCCCACTTCCTGACCGGCAAGCTGAGTGCTCCTGAGCTGACGGCGGCGCTGA
CGGATCAGCTGATGCTGTACCGCGACCAAACCGACCAGGCGATGCTGAACATGCTGGACT
CGCATGACACCGCGCGGCTGCTAACGGTGGCCGGCGGCGACGAGGACCTGGCCCTGCAGG
CGCTGGCCTTCACCTTCCTGCAAACCGGGATGCCGTGCCTGTACTATGGCACGGAAATGG
GCATGGCCGGAGAAAACGATCCCGACTGCCGGCGGCCAATGGACTGGGCCCAGCTGAAGG
GCCCGATTTGGCAGCGTGTGCAGGCCCTTGTGACCTTCCGCCGCGCCCAGTCGGCAACGC
TAGGCACCGGCACCACGGCGCTGAGCGTGACCGCAGCCGGGCTGCTTAAGGTAACCCGCA
CAGGTGAGCACACCGTGACGGCGTATTTTAACACCACCAAGCAGATGGCGACACTGACCG
TCAGTCCATTACTGGCGCAGGGTTACGCCGGCCAGCGGCTGGCGCCAACCGGGTTTGCTG
TTATGGTTCAGTAAGATTATGTTAGCGGTAACAGGCAATTTGACCTTTTAAAAGCGTTTT
CATATTATCATAATCAAAAGTGTAGAAAAGTTCAGGTGGCGCAATTCACCTCCCGAAAGT
GAAGGATGCAAGATGAAACGGATATTTGAAATCGACCCGTGGCTGGTGCAAAGCCACCAA
TTGAACCCCAACGAGAAACGCCTGCAGGAAAGCATGACCGCCATCGGCAACGGCTACATG
GGTCTGCGCGGTAACTTCGAAGAAGGTTACAGCGGTGATCACCTGCAAGGCACGTACCTC
GGCGGCGTCTGGTTCCCAGATAAAACCGTCGTCGGTTGGTGGAAAAACGGCTACCCGGAT
TACTTCGGCAAGGCGATCAACGCGCCGAGCTTCATCGGCATGGCGCTCACCGTGAACGGC
GAGCGCGTCGATCTGGCCACCAGCGTCTACCGCGATTTCACCCTCACGCTTGACCTGCAC
CAGGGCCTGCTGACCCGGAGCTTCGTGTTCGAGGGCAAAAAGGCCACGGTGCGCTTCACC
TTCAAGCGTTTCCTCAGCAACGTAATCAAGGAGGCGGCGCTGGTGCAGCTCACCGCCGAA
AGCCTTGTCGGACCGGCCGAGCTGACGGTGGCCGCACAGCTCGACGGCAACGTCACGAAC
GAGGACAGCAACTACGACGAGCGCTTCTGGGCACCGCAGGGGGAAAACGCCGCGGCAGGC
ACCATCCAGCTGCAGACCAAGCCCAACCCGTTCGGGGTCCCGCAGTTCACGGTGCTGCTC
AAGCAAAGCCTGCGCCAAGGGGCAACCCTTTTACCCGGCACCGTGACCACCAGCACCGGC
CAGCTGACCAGCACGGTCACGCTGCCGCTGGCGCCAAACGTGCCGGTCCAGCTGGAAAAG
GACGTCATCGTGGTCACGAGCCGCGACGTCGCCCCTGAGGCCCAGGCCGAAGCGGCCGCG
GAGCTGATGACACAGCTGCAGGGCCAAAGCTTTGCGGCCCAGCTGGCGGCACACACCGCC
CTGTGGGCCAAGCGCTGGGCCCAAAGCGACGTGGTGATTGAAGGCGACGACGCGGCCCAG
CAGGGGATCCGCTTCAACCTCGCCCAGCTGTTCATGACCTATTACGGCGACGATAAGCGG
CTCAACGTGGGGCCGAAGGGTTTCACCGGCGAGAAGTACGGCGGGGCGACCTACTGGGAC
ACCGAGGCGTACGTGGTGCCGATGTACGTCGCCGCCACCCCTCCGGCCGTGACCCGGGCA
CTGCTGCAGTACCGGCACGACCAGCTGCCCGGCGCCTACCACAACGCCCAGCAGCAGGGG
CTCAAAGGGGCCTTGTTCCCGATGGTGACCTTCAACGGCATCGAGTGCCACAATGAATGG
GAAATCACCTTCGAGGAGCTGCACCGTAACGCAGCGGTCGCCTTCGCGATTTACCAGTAC
ACGGCCTACACCGGCGATGAAAGCTACGTCAACCACGACGGCATGGAGGTGCTGGTGGGC
ATCAGCCGCTTCTGGGCGGACCGGGTCCACTTCTCCAAGCGCGCCGGCAAGTACATGATC
CACGGCGTCACCGGGCCGAACGAGTACGAAAACAACGTCAACAACAACTGGTACACCAAC
ACGATGGCCGCCTGGTGCCTGGAGTACACGCTGGCCCGGCTGCCGAAGGCCGATGCCGCC
ATTCAGGCCAAGCTGGCCGTGAGCGCCGCCGAGCAGCGCCAGTGGCAGGACATTATCGAC
CACATGTACTATCCGGAGGACAAGAAGCTGGGCATCTTCGTCCAGCACGACACCTTCCTG
GATAAGGACCTGCGGCCGGCAAGCTCGATTCCGGCCGACCAGCGGCCAATCAACCAGCAC
TGGTCCTGGGACCGAATCCTGCGGTCGCCGTTCATCAAGCAGGCGGATGTGCTCCAGGGC
CTGTACTTCCTGAACAATCGCTTCACCCGCGAGCAGAAGGAACGCAATTTTGACTTCTAC
GAGCCGCTGACGGTGCACGAAAGCTCGCTGAGTGCCTCGATTCACGCGGTGCTGGCCGCC
GAGCTCGGTAAGCAGGATAAGGCCGTTGAACTCTATCAGCGTACGGCTCGTCTGGACCTG
GACAACTACAACAACGATACGGCAGACGGTCTGCACATCACCTCGATGACCGGCGGCTGG
CTGGCTATCGTGCAGGGCTTCGCCGGCATGCGCTACGACCACGATCAGCTGCGGTTCGAT
CCGTTCCTGCCGAAGCAGTGGCAGGGTTACCAGTTCCGCATCAACTACCGCGGCCGGGTG
ATCCAGGTCGCGGTGGGGAAAACCGTTGCAGTGACCCTGCTGGCCGGCCCGCCGCTGACC
GTCATGGTTGCCGGCCAGCCGCAGCATTTGGAGGTGAGCGCGCATGCTTAAAGGATTGCT
GTTCGACCTCGACGGCGTCTTGACCGACTCGGCCAAGTTCCACCTGCAGGCCTGGAGCCA
GCTGGCCACCCAGCTGGGCATCACCCTGACGCCGGCCGAGCGCGAAGGCCTGCGCGGCCG
CTCGCGGCTGGACTCGCTGAACCTGATTTTGGCGGCAGGCGCCCAGGAAGACCGGTTCAG
TGCCGCAGAGAAAACGGCGCTAACCGACCAGAAGAACGCGGTGTACCTGAAGCTGATTCA
GACGATGACGCCGGTGGACATCCTGCCGGGCATTCCGCAACTGCTGAAGGACGCGCAGGC
GGCCGGCCTGAAAATGGCAATCGCCTCGGCGTCGCGGAACGCCCCGACAATTCTTGACCA
CCTGGGCCTGGCCGCCAGTTTCGACGCCATCGTCGATCCGGCGACCCTGCACCGCGGCAA
GCCCGACCCGGAGATCTACCAGCAGGCGCAAGCGCTGCTGGGGCTCCAGGCCGCCGAGGT
GATCGGCTTCGAGGATGCCTCGGCCGGGGTCGCCGCCATCAAAGCGGCCGGTCAGTTCGC
GGTTGGCATCGGGGATGCCCGGCTTCTGGCCGCAGCGGATTACCTAGTGAAAGACACGGC
GGCCCTGCAGCTGAGCCAGTTGCAAGCGGCGTTCGCCAAAGAAAGTGGGGAGACTAATGG
TTGAAATCGACTTGGACCACCTCTACAAGAAGTACGACGACGGCGAGGATTACTCGGTGG
TGGACTTCGACCTTCACATCAAGGATAAGGAGTTCATCGTGTTCGTCGGCCCCTCGGGCT
GCGGGAAGTCCACCACGCTGCGTATGATTGCGGGGCTGGAGGACATTACCAAAGGCGAGC
TGAAAATCGACGATAAGGTGATGAACGACGTGGCCCCCAAGGACCGCAACATCGCCATGG
TGTTTCAGAACTACGCCTTGTACCCGCACATGTCAGTGTACGACAACATGGCGTTCGGCC
TAAAGCTACGGCACTACAAGAAGGAGGACATCGACAAACGCGTGCAAAACGCGGCGGAGA
TCCTCGGCCTGAAGCCGTTTCTCGACCGGAAGCCGGCCGCCTTGTCCGGGGGCCAGCGGC
AGCGGGTGGCCTTGGGCCGGGCCATCGTCCGCGACGCCCCAATTTTCCTGATGGATGAGC
CGTTGTCGAACCTGGACGCGAAGCTGCGGGTGTCCATGCGGGCGGAAATCGCCAAGCTCC
ACCAGCGCCTGAACACCACCACGATTTACGTGACCCACGACCAAACCGAGGCCATGACTA
TGGCCGACCGGGTTGTCGTCATGTCCGTTGGCCACGTGCAGCAGATTGGCACCCCGGCCG
AGATTTACCAGAACCCGCGGAACCAGTTCGTGGCCGGGTTCATCGGGTCGCCGGCGATGA
ACTTCTTCAACATGACCTACCAGGACGGCTTCGTCAGCGACGGCCAAAGCATTCGCCTCA
AAGTGCCGGAAGGCCGGGCGAAGATTCTGGACGACCAAGGGTACAACGGCAAGGAAGTCG
TGTTCGGCATCCGGCCGGAGGACATCCATTCGGAGGAGGCCTTCCTGGAGACCTGGCCGG
ACGCGGTTATCAGCTCAACCGTGTCGGTGTCAGAGCTCCTGGGCGCCACCGAGCAGCTTT
ACCTGAAGGCGGATGACACCGAGTACGTTGCCAACGTCAACGCGCGCGACTTCCACAATC
CCGGGGATCATGTGAAAATGGGCTTCGACGTCAACAAGGCGCACTTCTTCAACAAGGACA
CGACCATGGCCATCGTGGCTAAGCCGATTCCGCTGGAAGGCTGAGGAGGTGAGTGCATGA
CCCCATGGTGGCAGCAAGCCGTCATTTACCAGATCTACCCGAAGAGTTTTCAGGACAGCA
ACGGGGATGGCATCGGCGATTTGCCGGGGATTACCAGTCGCCTTGATTACCTTAAGCGGC
TGGGCGTCGATGCCCTTTGGCTGAGCCCAGTGTATGTGTCGCCCGGCGAGGACAACGGCT
ACGACATCGCGGACTACGAGGCCATCGATCCCCAGTTCGGGACGATGGCCGACATGGACG
CCTTGATCGCCGCCGCCAAGCAGCGCG
SEQ ID 17
Position 2793833 . . . 2794809
CCCGCGATTTTGGCGTGATTGGCTTCGACGGGGTATTCCTGGACCAGGTGTCCAACCCCA
AGCTGACCACGGTGAAGCAGCCCGTGCAGCGCCTCGGCGAACTGCTGGCCCGCATGCTCC
TGCAGAAGGTGGCACAGTCCGGCGCCCAACAGGGGGAGCTGCTGGTCGATCCTGAGCTGA
TTGCTCGGGACACGACGCGAAAGTAGATCGGATTTCAACTGTCCTTACCGCTATGGTAGG
GCCAGTTTTTAGGCTCTATGTCAAATCTAATTCATAGCTAATAGTTGATTTGGCAACGCC
TAAGGCGTCAGCCATATCTTGGTAAGTATGATGGCCTTCACTGACCAGTTGAGCTAGCGC
ACCACGTTGAAAACGTGATAAAGTAGAAGTACCCAAAGTAATCACTCCTTATAGCTGGTT
GGAATTAACTACTCCATTGTAAGAGATTGCTTTGGGCCTTTTTTTATTTTTGTTCGGATT
AATTATAGAATTTGTCTAATTAGTTGAAAATTCTTAGGGTTGCCCATATATCTTTTAGTC
TGGTCATTAGTTTTTATGTTTGATCTGCTTTTTTCTGATCGCAAACACCCACAACTGCGA
GTGAGTCCTTTTTGAAGTCGTTGACTGTCAACAATACATTTATTTCCACATTGACATTGA
CAGAGCCAAAGCGCGTTGCCATTAGAACTGCTTCCAAAAAAGCTAATAACTGTGAGTCGC
CCAAACGTTTGATTAGCTAAATCGATACGCTTTTGCATACTAATCCTCCCGCTTGATAAG
AAGGTACTTAAATAGTTGCTTTCAATTGATCTAATCGCCATTGGCACCATGAAATAAAGG
CTAATTCGTCAATCTTTGGAATGCCATAGGTTCTAGCATACGTTAACTTTTGAGTGGTGA
GTAGTTGATCATAGGGTTTACTAATAATACCAACCACAAGGATATCGACTTTTTTGTCAA
TCCCGTTGACAGGCTTT
SEQ ID 18
Position 2967081 . . . 2968319
AAATACGCAAAAGAACCCGACGAGAGTTAAGTCTCATCGGGTTCTCAGTCGTGGATGAAT
TAGAAGCATTGTTAGCTGCATAACCTTCAACATAGGATCAATAGCTGGTTAGATGGTCAT
CTCTCAGACTGTTTGCACCAGATCCAGGCAAACGTGTTTATATCCTTGGTCATACTCAGG
ATAGATGGGCATTGTGAGTGCAACAGGACTTAGTTGCTTGTATGCTAGGCAATGTTGGCC
TTGATACAGAGGATCACTCTGTTTCTGATCTGGATGATAACCTCGTTTTTCCATTTCAGG
ACGCTGTCCCAATAATTGTTGCCCTGGGAGTTTGTCGAATTAAGTCTTGGTGCCACAAAC
ACACGCTACGGCTTCTTTTCTTGCAAACTGATCTTAGTATTTAGGGCAGTTGCATGATTA
CGGAATTGAACCATTTTATAATGAATCGTCTTTTCTATAAGTCTATAGAAAGTGCAGGTA
ATGGCATTTTCTCCAGATCGGATTGTCTAATCAATTTAATTGATTTTTTTGGTGTGTTTG
ATTATATTGCTTTTGCAAAGGTACAATATACCTTTTCTCTGCTGCCTTGCGAGCAGCGAT
GGCATCCTCCATATGAACATATACACGATTTAGGACAAGATGGCCTTGAAAGTACAGCCT
TGCGACCCACTTTTGAGCAGTTTTATCCCAACTAACTCCGATAACGCCAGATTTGTTATT
GGACCGTTTAAGTGTAGAAGCAACTAAATTTGTTCGATTAATTAGTTGAAAATTCTTGGG
ATTGCCCATGTATTTTTTAGTCTGGTTATTAGCTTTTATGTTTGATCTGCTTATTTCTGA
TCGCAAACACCCACAACTGCGAGTGAGTCCTTTTTGAAGTCGTTGACTGTCAACGATACA
TTTATTTCCACATTGACATTGACAGAGCCAAAGCGCGTTTCCATTAGAACTGCTTCCAAA
AAAACTAATGACTGTGAGTCGTCCAAACGTTTGATTGGCCAAATCGATACGCTTTTGCAT
ACTAATCTCCCCGTTTGATAAGAAGGTACTTAAAGAGTTGTTTTCAATTGATCTAGTCGC
CATTGGCACCATGAAATAAAGGCTAATTCGTCAATCTTTGGAATGCCATAGGTTCTAGCA
TACGTTAACTTTTGAGTGGTAAGTAGTTGATCATAGGGTTTGCTAATAATACCAACCACA
AGGATATCGACATTTTTGTCAATCCCGTTGACAGGTTTT

1. A method for the treatment and/or prevention of tumours, comprising administering to a subject in need thereof fermented supernatant, or fractions thereof, of the Lactobacillus casei or paracasei species, said species:
being the strain deposited according to the Budapest Treaty with No. CNCM I-5220 and/or
comprising in its DNA genome a DNA sequence essentially identical to one of the sequences selected from the group consisting of: SEQ ID No 1 to 5.

2. The method according to claim 1, wherein said fermented supernatant, or fractions thereof, potentiates an anti-tumour effect of an agent capable of inducing the CD8+ effector cells, said agent optionally being an immune checkpoint inhibitor (ICI),
and/or activate tumour immunity.

3. (canceled)

4. (canceled)

5. The method of claim 1, wherein said method further comprises administering an agent capable of inducing the CD8+ effector cells, said agent optionally being an immune checkpoint inhibitor (ICI).

6. (canceled)

7. The method of claim 5, wherein the agent is one or more selected from the group consisting of: anti-PD-1 antibody, anti-CTLA4 antibody, anti-PD-L1 antibody, a PD-1 antagonist and fragments thereof, single-chain antibodies and fusion proteins comprising fragments of the antibody, a CpG-oligonucleotide immunotherapeutic agent, cyclophosphamide, immunotherapy, vaccination, CAR-T or CAR-NK, chemotherapy, radiotherapy and any combination thereof and/or wherein the ICI is an inhibitor of a target selected from the group consisting of: CTLA4, PD-1, PDL-1, TIM-3, or LAG-3.

8. The method of claim 1, wherein said fermented supernatant is obtainable through a method comprising two fermentations of said Lactobacillus casei or paracasei species.

9. The method of claim 1 wherein said fermented supernatant is obtainable through a method comprising two fermentations of said Lactobacillus casei or paracasei species, at least one of which is carried out in a minimum solution added with prebiotic fibres, wherein the prebiotic fibres are fructooligosaccharides (FOS) and the minimum solution is saline.

10. The method of claim 1, wherein the fermented supernatant is obtainable by a method comprising the steps of:
a) growing an inoculum of the Lactobacillus strain in a suitable culture medium, at a temperature ranging from 4 to 40° C., to obtain a biomass and allowing the fermentation of said biomass to proceed for 12 to 36 hours to obtain a fermented biomass;
b) centrifuging said fermented biomass to obtain a fermented biomass pellet and a first fermented product;
c) incubating said fermented biomass pellet in a minimum solution and allowing a further fermentation for 12 to 36 hours at a temperature ranging from 4 to 40° C. to obtain a further fermented biomass; and
d) separating said further fermented biomass from a second fermented product by centrifugation
where the minimum solution is added with prebiotic fibres.

11. The method of claim 1, further comprising administering an anti-PD-1 antibody.

12. The method of claim 1, wherein Lactobacillus paracasei comprises in its DNA genome at least one DNA sequence essentially identical to SEQ ID No 6 to 18.

13. The method of claim 1 wherein said supernatant, or fractions thereof, is comprised in a pharmaceutical formulation or composition further comprising mannitol optionally at 1-30% (w/V), and wherein said composition or pharmaceutical formulation is optionally pulverized.

14. A composition comprising:
a) a fermented supernatant, or fractions thereof, of the Lactobacillus casei or paracasei species, said species:
being the strain deposited according to the Budapest Treaty with No. CNCM I-5220 and/or
comprising in its DNA genome a DNA sequence essentially identical to one of the sequences selected from the group consisting of: SEQ ID No 1 to 5; and
b) an agent capable of inducing the CD8+ effector cells.

15. The composition according to claim 14, wherein the agent capable of inducing the CD8+ effector cells is an immune checkpoint inhibitor (ICI), optionally an anti-PD-1 antibody, and/or an immunotherapy.

16. The composition according to claim 15, wherein the fermented supernatant, or fractions thereof is comprised in a pharmaceutical formulation or composition further comprising mannitol, or a pharmaceutical formulation thereof, at 1-30% (w/V), and wherein said composition or pharmaceutical formulation is optionally pulverized.

17. (canceled)

18. (canceled)

19. (canceled)

20. (canceled)

21. (canceled)

22. (canceled)

23. (canceled)

24. A method for potentiating an anti-tumour effect of an agent capable of inducing the CD8+ effector cells, comprising administering a fermented supernatant, or fractions thereof, of the Lactobacillus casei or paracasei species, said species:
being the strain deposited according to the Budapest Treaty with No. CNCM I-5220 and/or
comprising in its DNA genome a DNA sequence essentially identical to one of the sequences selected from the group consisting of: SEQ ID No 1 to 5, to a subject in need thereof.

25. A method for activating tumour immunity, comprising administering a fermented supernatant, or fractions thereof, of the Lactobacillus casei or paracasei species, said species:
being the strain deposited according to the Budapest Treaty with No. CNCM I-5220 and/or
comprising in its DNA genome a DNA sequence essentially identical to one of the sequences selected from the group consisting of: SEQ ID No 1 to 5, and an agent capable of inducing the CD8+ effector cells, to a subject in need thereof.

26. The method of claim 1, wherein said tumor is a solid tumor.

27. The method of claim 1, wherein said tumor is selected from the group consisting of breast cancer, melanoma, bladder cancer, head and neck cancer, Hodgkin's lymphoma, kidney cancer, and non-small cell lung cancer.
